(19) United States
(12) Patent Application Publication
(10) Pub. No.: US 2024/0058129 A1
(43) Pub. Date: Feb. 22, 2024

(54) MEDICAL BALLOON SENSING ASSEMBLY AND METHOD

(71) Applicant: EDWARDS LIFESCIENCES CORPORATION, Irvine, CA (US)

(72) Inventors: Shaun Fetherston, Coto de Caza, CA (US); Alexander H. Siemons, Yorba Linda, CA (US); Blake W. Axelrod, Sierra Madre, CA (US); Gil Senesh, Laguna Beach, CA (US); Tomer Saar, Pardes Hanna-Karkur (IL); Oren Cohen, Kadima (IL); Elazar Levi Schwarcz, Netanya (IL); Tamir S. Levi, Zikhron Yaakov (IL); Michael Bukin, Pardes Hanna (IL); Michael R. Bialas, Lake Forest, CA (US); Timothy A. Geiser, Laguna Niguel, CA (US); Sudarsan Pranatharthikaran, Costa Mesa, CA (US); Carla Susana Cerqueira, Watchung, NJ (US)

(21) Appl. No.: 18/112,455

(22) Filed: Feb. 21, 2023

Related U.S. Application Data
(63) Continuation of application No. PCT/US2021/047443, filed on Aug. 25, 2021.
(60) Provisional application No. 63/165,294, filed on Mar. 24, 2021.
(60) Provisional application No. 63/069,842, filed on Aug. 25, 2020.

(51) Int. Cl. A61F 2/24; A61B 8/00; A61B 8/08

(52) U.S. Cl. CPC A61F 2/2496; A61B 8/445; A61B 8/0883; A61F 2/2433; A61B 2090/064

(57) ABSTRACT

A medical balloon sensing assembly constituted of: a handle; a first catheter extending distally from the handle; an inflatable medical balloon, the inflatable medical balloon secured by the first catheter; at least one sensor position member; and at least one sensor secured to the at least one sensor position member, wherein the at least one sensor position member is arranged to contact an outer face of the inflatable medical balloon.

### CROSS-REFERENCE TO RELATED APPLICATIONS

This application is a continuation of International Application No. PCT/US2021/047443, filed Aug. 25, 2021, which claims benefit of U.S. Provisional Application No. 63/069,842, filed on Aug. 25, 2020, and U.S. Provisional Application No. 63/165,294, filed on Mar. 24, 2021, the contents of each of which are herein incorporated by reference in their entirety.

### TECHNICAL FIELD

The invention relates generally to the field of medical balloons, and more specifically to a medical balloon sensing assembly and method.

### BACKGROUND OF THE INVENTION

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

When implanting a prosthetic valve, such as a balloon expandable valve, it is desirable to expand the valve to a maximum size allowed by the patient's anatomical considerations, in order to avoid paravalvular leakage or other unfavorable hemodynamic phenomena across the valve that may be associated with a mismatch between the valve's expansion diameter and the surrounding tissue, while mitigating the risk of annular rupture that may result from over-expansion. Additionally, it is desirable to avoid applying excessive force to calcifications present in the artery.

### SUMMARY

Accordingly, it is a principal object of the present invention to overcome at least some of the disadvantages of prior art inflatable medical balloon devices. This is accomplished in one example by a medical balloon sensing assembly, comprising: a handle; a first catheter extending distally from the handle; an inflatable medical balloon, the inflatable medical balloon secured by the first catheter; at least one sensor position member; and at least one sensor secured to the at least one sensor position member, wherein the at least one sensor position member is arranged to contact an outer face of the inflatable medical balloon.

In one example, the at least one sensor position member at least partially circumferentially surrounds the outer face of the inflatable medical balloon. In another example, the at least one sensor position member comprises a sleeve, the sleeve covering the outer face of the inflatable medical balloon.

In one example, the at least one sensor position member comprises at least one elongated arm. In one further example, the at least one elongated arm comprises a plurality of elongated arms.

In one example, the at least one sensor position member comprises a collapsible stent. In another example, the medical balloon sensing assembly further comprises a second catheter extending distally from the handle, the at least one sensor position member secured to the second catheter.

In one example, the medical balloon sensing assembly further comprises a prosthetic valve, the inflatable medical balloon positioned within the prosthetic valve such that the prosthetic valve expands responsive to an inflation of the inflatable medical balloon. In one further example, the at least one sensor position member is secured between the inflatable medical balloon and the prosthetic valve.

In another example, the at least one sensor comprises at least one force sensor. In one further example, the medical balloon sensing assembly further comprises a sensor data unit in communication with the at least one force sensor, wherein the at least one force sensor comprises a plurality of force sensors, wherein the sensor data unit is arranged, responsive to an output of the plurality of force sensors, to: generate a map of forces applied to the inflatable medical balloon; and output an indication of the determined map.

In one example, the medical balloon sensing assembly further comprises a sensor data unit in communication with the at least one sensor, wherein the at least one sensor comprises a plurality of force sensors, wherein the sensor data unit is arranged, responsive to an output of the plurality of force sensors, to: generate a map of forces applied to the inflatable medical balloon; responsive to the determined map of forces, determine an appropriate orientation for a prosthetic heart valve; and output an indication of the determined orientation.

In another example, the medical balloon sensing assembly further comprises a sensor data unit in communication with the at least one sensor, wherein the at least one sensor comprises a plurality of force sensors, and wherein the sensor data unit is arranged, responsive to an output of the plurality of force sensors, to: generate a map of forces applied to the inflatable medical balloon; responsive to the determined map of forces, determine whether implantation of a balloon expandable prosthetic valve at an anatomical location of the inflatable medical balloon is viable; and output an indication of the determined viability.

In one example, the at least one sensor comprises a plurality of flex sensors. In one further example, the medical balloon sensing assembly further comprises a sensor data unit in communication with the plurality of flex sensors, and wherein the sensor data unit is arranged, responsive to an output of the plurality of flex sensors, to: determine a surface topography of the inflatable medical balloon; and output an indication of the determined surface topography.

In another example, the at least one sensor comprises: a diameter sensor, an output of the diameter sensor responsive to a radial diameter of the inflatable medical balloon; and a sensor data unit in communication with the diameter sensor, wherein, responsive to the output of the diameter sensor, the control circuitry is arranged to: determine a diameter indication of the inflatable medical balloon; and output the determined diameter indication. In one further example, the determined diameter indication comprises a change in a radial diameter of the inflatable medical balloon.

In another further example, the diameter sensor comprises: at least one radially translatable member juxtaposed with a surface of the inflatable medical balloon such that the inflation of the inflatable medical balloon radially translates the at least one radially translatable member; and a linear displacement sensor coupled to the at least one radially translatable member and in communication with the sensor data unit, an output of the linear displacement sensor configured to be responsive to the radial translation of the at least one radially translatable member. In one yet further example, the at least one radially translatable member is circumferentially positioned on the outer face of the inflatable medical balloon.

In one further example, the diameter sensor comprises a strain gauge circumferentially position on the outer face of the inflatable medical balloon. In another further example, the diameter indication comprises an indication of the recoil of the inflatable medical balloon.

In one example, the medical balloon sensing assembly further comprises at least one ultrasound transducer positioned on the catheter within the inflatable medical balloon. In one further example, the at least one ultrasound transducer is directed at a surface of the inflatable medical balloon. In one yet further example, the at least one ultrasound transducer comprises at least two ultrasound transducers, an orientation of a first of the ultrasound transducers generally opposing an orientation of the second of the ultrasound transducers.

In another example, the medical balloon sensing assembly further comprises: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein, responsive to the output of the at least one force sensor, the pump is arranged to adjust the flow of the inflation fluid.

In one independent example, a medical balloon sensing assembly is provided, the medical balloon sensing assembly comprising: a handle; a first catheter extending distally from the handle; an inflatable medical balloon secured by the first catheter; and at least one ultrasound transducer positioned on the first catheter within the inflatable medical balloon.

In one example, the at least one ultrasound transducer is directed at a surface of the inflatable medical balloon. In another example, the at least one ultrasound transducer comprises at least two ultrasound transducers, an orientation of a first of the ultrasound transducers generally opposing an orientation of the second of the ultrasound transducers.

In one example, the medical balloon sensing assembly further comprises a sensor data unit in communication with the at least one ultrasound transducer, wherein the sensor data unit is arranged, responsive to an output of the at least one ultrasound transducer, to: determine a diameter indication of the inflatable medical balloon; and output an indication of the determined diameter indication. In one further example, the diameter indication comprises a radial diameter of the inflatable medical balloon. In another example, the diameter indication comprises a surface topography of the inflatable medical balloon.

In one further example, the diameter indication comprises a protrusion depth of one or more protrusions extending into the inflatable medical balloon. In another further example, the diameter indication comprises a height-diameter aspect ratio of one or more protrusions extending into the inflatable medical balloon. In another further example, the diameter indication comprises and indication of the recoil of the inflatable medical balloon.

In one further example, the sensor data unit is further arranged, responsive to the determined diameter indication, to: determine an appropriate orientation for a prosthetic heart valve; and output an indication of the determined orientation. In another further example, the sensor data unit is further arranged, responsive to the determined diameter indication, to: determine whether implantation of a balloon prosthetic valve at an anatomical location of the inflatable medical balloon is viable; and output an indication of the determined viability.

In one example, the medical balloon sensing assembly further comprises a prosthetic valve, the inflatable medical balloon positioned within the prosthetic valve such that the prosthetic valve expands responsive to an inflation of the inflatable medical balloon. In another example, the medical balloon sensing assembly further comprises: at least one sensor position member; and at least one sensor secured to the at least one sensor position member, wherein the at least one sensor position member is arranged to contact an outer face of the inflatable medical balloon.

In one example, the medical balloon sensing assembly further comprises: at least one sensor position member; a plurality of force sensors secured to the at least one sensor position member; and a sensor data unit in communication with the plurality of force sensors, wherein the at least one sensor position member is arranged to contact an outer face of the inflatable medical balloon, and wherein the sensor data unit is arranged, responsive to an output of the plurality of force sensors, to: generate a map of forces applied to the inflatable medical balloon; and output an indication of the determined map. In another example, the medical balloon sensing assembly further comprises at least one sensor, wherein the inflatable medical balloon exhibits at least one depression, the at least one sensor positioned within the at least one depression.

In one example, the medical balloon sensing assembly further comprises: at least one force sensor juxtaposed with the inflatable medical balloon; and a sensor data unit in communication with the at least one sensor, wherein, responsive to the output of the at least one force sensor, the sensor data unit is arranged to: determine a force applied to the inflatable medical balloon; and output an indication of the determined force.

In another independent example, a medical balloon sensing assembly is provided, the medical balloon sensing assembly comprising: a handle; a catheter extending distally from the handle; an inflatable medical balloon exhibiting at least one depression, the inflatable medical balloon secured by the catheter; and at least one sensor positioned within the at least one depression.

In one example, the medical balloon sensing assembly further comprises: a sensor data unit; and at least one communication medium, the at least one sensor in communication with the sensor data unit via the at least one communication medium, wherein the inflatable medical balloon further exhibits at least one channel, the at least one communication medium positioned within the at least one channel. In another example, the medical balloon sensing assembly further comprises an outer layer encompassing an outer face of the inflatable medical balloon, the at least one sensor facing an inner face of the outer layer.

In one example, the at least one sensor comprises at least one force sensor. In another example, the medical balloon sensing assembly further comprises a sensor data unit in communication with the at least one sensor, wherein the at least one sensor comprises a plurality of force sensors, and wherein the sensor data unit is arranged, responsive to an output of the plurality of force sensors, to: generate a map of forces applied to the inflatable medical balloon; and output an indication of the determined map.

In one further example, responsive to the determined map of forces, the sensor data unit is further arranged to: determine an appropriate orientation for a prosthetic heart valve; and output an indication of the determined orientation. In another further example, responsive to the determined map of forces, the sensor data unit is further arranged to: determine whether implantation of a balloon expandable prosthetic valve at an anatomical location of the inflatable medical balloon is viable; and output an indication of the determined viability.

In one example, the at least one sensor comprises a plurality of flex sensors, each of the plurality of flex sensors juxtaposed with the outer face of the inflatable medical balloon at a respective predetermined location. In another example, the medical balloon sensing assembly further comprises a sensor data unit in communication with the at least one sensor, wherein the at least one sensor comprises a plurality of flex sensors, each of the plurality of flex sensors juxtaposed with the outer face of the inflatable medical balloon at a respective predetermined location, and wherein the sensor data unit is arranged, responsive to an output of the plurality of flex sensors, to: determine a surface topography of the inflatable medical balloon; and output an indication of the determined surface topography.

In one example, the at least one sensor comprises: a diameter sensor, an output of the diameter sensor responsive to a radial diameter of the inflatable medical balloon; and a sensor data unit in communication with the diameter sensor, wherein, responsive to the output of the diameter sensor, the control circuitry is arranged to: determine a diameter indication of the inflatable medical balloon; and output the determined diameter indication. In one further example, the determined diameter indication comprises a change in a radial diameter of the inflatable medical balloon.

In another further example, the diameter sensor comprises: at least one radially translatable member juxtaposed with an outer face of the inflatable medical balloon such that the inflation of the inflatable medical balloon radially translates the at least one radially translatable member; and a linear displacement sensor coupled to the at least one radially translatable member and in communication with the sensor data unit, an output of the linear displacement sensor configured to be responsive to the radial translation of the at least one radially translatable member. In one yet further example, the at least one radially translatable member is circumferentially positioned on the outer face of the inflatable medical balloon.

In one example, the diameter sensor comprises a strain gauge circumferentially position on the outer face of the inflatable medical balloon. In another example, the medical balloon sensing assembly further comprises at least one ultrasound transducer positioned on the catheter within the inflatable medical balloon.

In one further example, the at least one ultrasound transducer is directed at a surface of the inflatable medical balloon. In another further example, the at least one ultrasound transducer comprises at least two ultrasound transducers, an orientation of a first of the ultrasound transducers generally opposing an orientation of the second of the ultrasound transducers.

In one example, the medical balloon sensing assembly further comprises: a reservoir containing a predetermined volume of inflation fluid, and a pump in fluid communication with the reservoir and the inflatable medical balloon, wherein the pump is arranged to generate flow of the inflation fluid into the inflatable medical balloon, and wherein, responsive to the output of the at least one force sensor, the pump is arranged to adjust the flow of the inflation fluid.

In one independent example, a medical balloon sensing assembly is provided, the medical balloon sensing assembly comprises: a handle; a first catheter extending distally from the handle; an inflatable medical balloon, the inflatable medical balloon secured by the first catheter; and a plurality of flex sensors juxtaposed with a surface of the inflatable medical balloon.

In one example, the medical balloon sensing assembly further comprises a sensor data unit in communication with the plurality of flex sensors, the sensor data unit arranged, responsive to an output of the plurality of flex sensors, to: determine a surface topography of the inflatable medical balloon; and output an indication of the determined surface topography. In another example, the medical balloon sensing assembly further comprises a sensor data unit in communication with the plurality of flex sensors, the sensor data unit arranged, responsive to an output of the plurality of flex sensors, to: determine a protrusion depth of one or more protrusions extending into the inflatable medical balloon; and output an indication of the determined protrusion depth.

In one example, the medical balloon sensing assembly further comprises a sensor data unit in communication with the plurality of flex sensors, the sensor data unit arranged, responsive to an output of the plurality of flex sensors, to: determine a height-diameter aspect ratio of one or more protrusions extending into the inflatable medical balloon; and output an indication of the determined ratio. In another example, the medical balloon sensing assembly further comprises at least one sensor position member arranged to contact an outer face of the inflatable medical balloon, the plurality of flex sensors secured to the at least one sensor position member.

In one further example, the at least one sensor position member comprises at least one elongated arm. In another further example, the medical balloon sensing assembly further comprises a second catheter extending distally from the handle, the at least one sensor position member secured to the second catheter.

In one example, the medical balloon sensing assembly further comprise: a diameter sensor, an output of the diameter sensor responsive to a radial diameter of the inflatable medical balloon; and a sensor data unit in communication with the diameter sensor, wherein, responsive to the output of the diameter sensor, the control circuitry is arranged to: determine a diameter indication of the inflatable medical balloon; and output the determined diameter indication. In another example, the medical balloon sensing assembly further comprises at least one ultrasound transducer positioned on the first catheter within the inflatable medical balloon.

In one further example, the at least one ultrasound transducer is directed at a surface of the inflatable medical balloon. In another further example, the at least one ultrasound transducer comprises at least two ultrasound transducers, an orientation of a first of the ultrasound transducers generally opposing an orientation of the second of the ultrasound transducers.

In one example, the medical balloon sensing assembly further comprises: a reservoir containing a predetermined volume of inflation fluid; and a pump in fluid communication with the reservoir and the inflatable medical balloon, wherein the pump is arranged to generate flow of the inflation fluid into the inflatable medical balloon.

In another independent example, a process for preparing an inflatable medical balloon is provided, the process comprising: inserting a predetermined volume of a polymer composition into a cavity of a mold, the mold exhibiting at least one protrusion or at least one depression; applying a predetermined pressure, at a predetermined temperature, to the inserted polymer composition to conform to the mold, thereby forming the inflatable medical balloon with at least one depression; removing the inflatable medical balloon from the mold; and positioning at least one sensor in the at least one depression of the inflatable medical balloon.

In one example, at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped. In another example, at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

In one example, the polymer composition is shaped as a tube. In another example, the polymer composition is in a molten state, the inserting comprising injecting the molten polymer composition into the cavity of the mold.

In one example, the process further comprises forming an outer layer encompassing an outer face of the inflatable medical balloon. In one further example, the at least one sensor faces the outer layer.

In one independent example, a process for preparing an inflatable medical balloon is provided, the process comprising: coating a mold with a polymer composition or emulsion, the mold exhibiting at least one protrusion or at least one depression; drying and/or curing the polymer coating, thereby forming the inflatable medical balloon with at least one depression; removing the inflatable medical balloon from the mold; and positioning at least one sensor in the at least one depression of the inflatable medical balloon

In one example, at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being channel shaped.

In another example, at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

In one example, the coating the mold comprises dipping the mold within the polymer composition or emulsion. In another example, the process further comprises forming an outer layer encompassing an outer face of the inflatable medical balloon. In one yet further example, the at least one sensor faces the outer layer.

In another independent example, an inflatable medical balloon is provided, the inflatable medical balloon prepared by a process comprising: inserting a predetermined volume of a polymer composition into a cavity of a mold, the mold exhibiting at least one protrusion or at least one depression; applying a predetermined pressure, at a predetermined temperature, to the inserted polymer composition to conform to the mold, thereby forming the inflatable medical balloon with at least one depression; removing the inflatable medical balloon from the mold; and positioning at least one sensor in the at least one depression of the inflatable medical balloon.

In one example, at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped.

In another example, at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being channel shaped.

In one example, the polymer composition is shaped as a tube. In another example, the polymer composition is in a molten state, the inserting comprising injecting the molten polymer composition into the cavity of the mold. In another example, the inflatable medical balloon, wherein the process further comprises further comprising forming an outer layer encompassing an outer face of the inflatable medical balloon. In one further example, the at least one sensor faces the outer layer.

In one independent example, an inflatable medical balloon is provided, the inflatable medical balloon prepared by a process comprising: coating a mold with a polymer composition or emulsion, the mold exhibiting at least one protrusion or at least one depression; drying and/or curing the polymer coating, thereby forming the inflatable medical balloon with at least one depression; removing the inflatable medical balloon from the mold; and positioning at least one sensor in the at least one depression of the inflatable medical balloon.

In one example, at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped.

In another example, at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

In one example, the coating the mold comprises dipping the mold within the polymer composition or emulsion. In another example, the inflatable medical balloon further comprises forming an outer layer encompassing an outer face of the inflatable medical balloon. In one yet further example, the at least one sensor faces the outer layer.

In another independent example, a medical balloon sensing method is provided, the method comprising: delivering an inflatable medical balloon and a first catheter to a predetermined anatomical location, the inflatable medical balloon secured to the first catheter; and inflating the delivered inflatable medical balloon, wherein at least one sensor position member is arranged to contact an outer face of the inflatable medical balloon, and wherein at least one sensor is secured to the at least one sensor position member.

In one example, the at least one sensor position member at least partially circumferentially surrounds the outer face of the inflatable medical balloon. In another example, the at least one sensor position member comprises a plurality of elongated arms.

In one example, the method further comprises delivering a second catheter to the predetermined anatomical location, the at least one sensor position member secured to the second catheter. In another example, the method further comprises delivering a prosthetic valve to the predetermined anatomical location, the inflatable medical balloon positioned within the prosthetic valve such that the prosthetic valve expands responsive to the inflation of the inflatable medical balloon. In one further example, the at least one sensor position member is secured between the inflatable medical balloon and the prosthetic valve.

In one example, the at least one sensor comprises a plurality of force sensors, the method further comprising, responsive to an output of the plurality of force sensors: determining a map of forces applied to the inflatable medical balloon; and outputting an indication of the determined map. In another example, the at least one sensor comprises a plurality of force sensors, the method further comprising, responsive to an output the plurality of force sensors: determining a map of forces applied to the inflatable medical balloon, responsive to the determined map of forces, determining an appropriate orientation for a prosthetic heart valve; and outputting an indication of the determined orientation.

In one example, the at least one sensor comprises a plurality of force sensors, the method further comprising, responsive to an output the plurality of force sensors: determining a map of forces applied to the inflatable medical balloon; responsive to the determined map of forces, determining a viability of implanting a balloon expandable prosthetic valve at the predetermined anatomical location; and outputting an indication of the determined viability.

In one independent example, a medical balloon sensing method is provided, the method comprising: delivering an inflatable medical balloon and a first catheter to a predetermined anatomical location, the inflatable medical balloon secured to the first catheter; and inflating the delivered inflatable medical balloon, wherein at least one ultrasound transducer is positioned on the first catheter within the inflatable medical balloon.

In one example, the at least one ultrasound transducer is directed at a surface of the inflatable medical balloon. In another example, the at least one ultrasound transducer comprises two ultrasound transducers, an orientation of a first of the ultrasound transducers generally opposing an orientation of the second of the ultrasound transducers.

In one example, the method further comprises, responsive to an output of the at least one ultrasound transducer: determining a diameter indication of the inflatable medical balloon; and outputting an indication of the determined diameter indication. In one further example, the diameter indication comprises a radial diameter of the inflatable medical balloon. In another further example, the diameter indication comprises a surface topography of the inflatable medical balloon. In another further example, the diameter indication comprises an indication of the recoil of the inflatable medical balloon.

In one further example, the diameter indication comprises a protrusion depth of one or more protrusions extending into the inflatable medical balloon. In another further example, the diameter indication comprises a height-diameter aspect ratio of one or more protrusions extending into the inflatable medical balloon.

In one example, the method further comprises, responsive to the determined diameter indication: determining an appropriate orientation for a prosthetic heart valve; and outputting an indication of the determined orientation. In another example, the method further comprises, responsive to the determined diameter indication: determining whether implantation of a balloon prosthetic valve at the predetermined anatomical location is viable; and outputting an indication of the determined viability.

In another independent example, a medical balloon sensing method is provided, the method comprising: delivering an inflatable medical balloon and a first catheter to a predetermined anatomical location, the inflatable medical balloon secured to the first catheter; and inflating the delivered inflatable medical balloon, wherein the inflatable medical balloon exhibits at least one depression, at least one sensor position within the at least one depression.

In one example, the inflatable medical balloon further exhibits at least one channel, at least one communication medium positioned within the at least one channel, the at least one sensor in communication with a sensor data unit via the at least one communication medium. In another example, an outer layer at least partially encompasses an outer face of the inflatable medical balloon, the at least one sensor facing an inner face of the outer layer.

In one independent example, a medical balloon sensing method is provided, the method comprising: delivering the inflatable medical balloon and a first catheter to a predetermined anatomical location, the inflatable medical balloon secured to the first catheter; and inflating the delivered inflatable medical balloon, wherein a plurality of flex sensors are juxtaposed with a surface of the inflatable medical balloon. In one further example, the method further comprises, responsive to an output of the plurality of flex sensors: determining a surface topography of the inflatable medical balloon; and outputting an indication of the determined surface topography. In another further example, the method further comprises, responsive to an output of the plurality of flex sensors: determining a protrusion depth of one or more protrusions extending into the inflatable medical balloon; and outputting an indication of the determined protrusion depth.

In one further example, the method further comprises, responsive to an output of the plurality of flex sensors: determining a height-diameter aspect ratio of one or more protrusions extending into the inflatable medical balloon, and outputting an indication of the determined ratio.

In another independent example, a medical balloon sensing assembly is provided, the medical balloons sensing assembly comprising: a handle; a catheter extending distally from the handle; an inflatable medical balloon secured by the catheter; at least one thermal sensor juxtaposed with the inflatable medical balloon; and a sensor data unit in communication with the at least one thermal sensor, wherein the sensor data unit is arranged, responsive to an output of the at least one thermal sensor, to: determine an indication of a rate of thermal dispersion between the inflatable medical balloon and a plurality of predetermined locations juxtaposed with the inflatable medical balloon; and output the determined indication of the rate of thermal dispersion.

In one example, the plurality of predetermined locations constitutes a predetermined area surrounding the inflatable medical balloon. In another example, the sensor data unit is further arranged, responsive to the determined indication of the rate of thermal dispersion, to: generate a map of the rate of thermal dispersion between the inflatable medical balloon and the plurality of predetermined locations; and output an indication of the determined map. In another example, the sensor data unit is further arranged, responsive to the determined indication of the rate of thermal dispersion, to: identify a tissue gap at one of the plurality of predetermined locations; and output an indication of the identified tissue gap.

In one further example, the medical balloon sensing assembly further comprises: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein the sensor data unit is further arranged, responsive to the identification of a tissue gap, to control the pump to increase an amount of the inflation fluid pumped into the inflatable medical balloon.

In one example, the sensor data unit is further arranged, for each of the plurality of predetermined locations and responsive to the respective determined indication of the rate of thermal dispersion, to: determine an extent of calcification at the respective predetermined location; and output an indication of the determined extent of calcification. In another example, the sensor data unit is further arranged, responsive to the determined indications of the rate of thermal dispersion, to: determine an appropriate orientation for a prosthetic heart valve; and output an indication of the determined orientation.

In one example, the sensor data unit is further arranged, responsive to the determined indications of the rate of thermal dispersion, to: determine whether implantation of a balloon expandable prosthetic valve at an anatomical location of the inflatable medical balloon is viable; and output an indication of the determined viability. In another example, the medical balloon sensing assembly further comprises a plurality of heating elements, each of the plurality of heating elements secured in relation to a respective predetermined point on a surface of the inflatable medical balloon, wherein the sensor data unit is further arranged to control the plurality of heating elements to generate heat at a predetermined temperature, the determined indication of the rate of thermal dispersion being between the plurality of heating elements and the plurality of predetermined locations.

In one further example, the at least one thermal sensor comprises a plurality of thermal sensors, each of the plurality of thermal sensors associated with a respective one of the plurality of heating elements, and wherein, for each of the plurality predetermined locations, the determination of the indication of the rate of thermal dispersion is responsive to a temperature difference between a respective one of the plurality of thermal sensors and the associated heating element.

In one example, the at least one thermal sensor comprises a plurality of thermal sensors, wherein, responsive to the sensor data unit, each of the plurality of thermal sensors is arranged to alternately operate in a heating mode and a sensing mode, wherein in the heating mode the respective thermal sensor is arranged to generate heat at a predetermined temperature, and in the sensing mode the respective thermal sensor is arranged to sense a temperature thereat, wherein each of the plurality of thermal sensors is associated with another or others of the plurality of thermal sensors, and wherein the sensor data unit is further arranged, at each of a plurality of predetermined time points, to: control a respective one of the plurality of thermal sensors to operate in the heating mode; and control the thermal sensors associated with the respective thermal sensor to operate in the sensing mode, wherein the determination of the indication of the rate of thermal dispersion is responsive to a temperature difference between the respective thermal sensor operating in the heating mode and the associated thermal sensors operating in the sensing mode.

In another example, the medical balloon sensing assembly further comprises: a plurality of imaging markers; and an imager in communication with the sensor data unit, wherein the imager is arranged to image the plurality of imaging markers, and wherein the sensor data unit is further arranged, responsive to the output of the imager to: determine a surface topography of the plurality of predetermined locations; and generate a 4-dimensional (4D) map of the plurality of predetermined locations, the 4D map comprising the determined surface topography and the respective indications of the rate of thermal dispersion.

In one example, the medical balloon sensing assembly further comprises: a plurality of imaging markers juxtaposed with a surface of the inflatable medical balloon; and an optical camera in communication with the sensor data unit, wherein the optical camera is arranged to image the plurality of imaging markers, and wherein the sensor data unit is further arranged, responsive to the output of the optical camera to: determine a surface topography of the plurality of predetermined locations; and generate a 4-dimensional (4D) map of the plurality of predetermined locations, the 4D map comprising the determined surface topography and the respective indications of the rate of thermal dispersion.

In another example, the medical balloon sensing assembly further comprises: a plurality of imaging markers; and a thermal camera in communication with the sensor data unit, wherein the thermal camera is arranged to image the plurality of imaging markers, and wherein the sensor data unit is further arranged, responsive to the output of the thermal camera to: determine a surface topography of the plurality of predetermined locations; and generate a 4-dimensional (4D) map of the plurality of predetermined locations, the 4D map comprising the determined surface topography and the respective indications of the rate of thermal dispersion.

In one example, a temperature of an interior of the inflatable medical balloon is less than 21 degrees Celsius. In another example, the medical balloon sensing assembly further comprises: a reservoir containing a predetermined volume of inflation fluid, the inflation fluid exhibiting a temperature of less than 21 degrees Celsius, wherein the reservoir is in fluid communication with the inflatable medical balloon. In one further example, the medical balloon sensing assembly further comprises a pump in fluid communication with the reservoir and the inflatable medical balloon, wherein the pump is arranged to generate flow of the inflation fluid into the inflatable medical balloon.

In one independent example, a medical balloon sensing method is provided, the method comprising: delivering an inflatable medical balloon and a catheter to a predetermined anatomical position, the inflatable medical balloon secured to the catheter; inflating the delivered inflatable medical balloon; determining an indication of a rate of thermal dispersion between the inflatable medical balloon and a plurality of predetermined locations juxtaposed with the inflatable medical balloon; and outputting the determined indication of the rate of thermal dispersion.

In one example, the plurality of predetermined locations constitutes a predetermined area surrounding the inflatable medical balloon. In another example, the method further comprises: responsive to the determined indication of the rate of thermal dispersion, generating a map of the rate of thermal dispersion between the inflatable medical balloon and the plurality of predetermined locations; and outputting an indication of the determined map.

In one example, the method further comprises responsive to the determined indication of the rate of thermal dispersion, identifying a tissue gap at one of the plurality of predetermined locations. In one further example, the method further comprises: generating flow of an inflation fluid into the inflatable medical balloon; and responsive to the identification of a tissue gap, increasing an amount of the inflation fluid pumped into the inflatable medical balloon. In another example, the method further comprises, for each of the plurality of predetermined locations and responsive to the respective determined indication of the rate of thermal dispersion: determining an extent of calcification at the respective predetermined location; and outputting an indication of the determined extent of calcification.

In one example, the method further comprises, responsive to the determined indications of the rate of thermal dispersion: determining an appropriate orientation for a prosthetic heart valve; and outputting an indication of the determined orientation. In another example, the method further comprises, responsive to the determined indications of the rate of thermal dispersion: determining whether implantation of a balloon expandable prosthetic valve at an anatomical location of the inflatable medical balloon is viable, and outputting an indication of the determined viability.

In one example, the method further comprises controlling a plurality of heating elements to generate heat at a predetermined temperature, the plurality of heating elements secured in relation to a respective predetermined point on a surface of the inflatable medical balloon, wherein the determined indication of the rate of thermal dispersion being between the plurality of heating elements and the plurality of predetermined locations. In one further example, for each of the plurality predetermined locations, the determination of the indication of the rate of thermal dispersion is responsive to a temperature difference between a respective one of a plurality of thermal sensors and an associated one of the plurality of heating elements.

In another example, the method further comprises, at each of a plurality of predetermined time points: controlling a respective one of a plurality of thermal sensors to operate in a heating mode wherein the respective thermal sensor is arranged to generate heat at a predetermined temperature, each of the plurality of thermal sensors associated with another or others of the plurality of thermal sensors; and controlling the thermal sensors associated with the respective thermal sensor to operate in a sensing mode wherein the associated thermal sensors are each arranged to sense a temperature thereat, wherein the determination of the indication of the rate of thermal dispersion is responsive to a temperature difference between the respective thermal sensor operating in the heating mode and the associated thermal sensors operating in the sensing mode.

In one example, the method further comprises: imaging a plurality of imaging markers juxtaposed with a surface of the inflatable medical balloon; responsive to the imaging, determining a surface topography of the plurality of predetermined locations; and generating a 4-dimensional (4D) map of the plurality of predetermined locations, the 4D map comprising the determined surface topography and the respective indications of the rate of thermal dispersion. In another example, a temperature of an interior of the inflatable medical balloon is less than 21 degrees Celsius.

In one example, the method further comprises generating flow of an inflation fluid into the inflatable medical balloon, the inflation fluid exhibiting a temperature of less than 21 degrees Celsius.

In another independent example, a medical balloon sensing assembly is provided, the medical balloon sensing assembly comprises: a handle; a plurality of catheters extending distally from the handle; a plurality of first inflatable medical balloons, each of the first inflatable medical balloons secured by a respective one of the plurality catheters, wherein the plurality of first inflatable medical balloons are arranged in a radially arrayed configuration.

In one example, each of the plurality of first inflatable medical balloons comprises a respective longitudinal axis, the longitudinal axes of the plurality of first inflatable medical balloons being generally in parallel with each other. In another example, each first inflatable medical balloon is arranged to contact each of a pair of first inflatable medical balloons adjacent thereto.

In one example, the medical balloon sensing assembly further comprises a second inflatable medical balloon secured by a respective one of the plurality of catheters, wherein the plurality of first inflatable medical balloons are radially arrayed about the second inflatable medical balloon. In one further example, each of the plurality of first inflatable medical balloons comprises a respective longitudinal axis and the second inflatable medical balloon comprises a respective longitudinal axis, and wherein the longitudinal axes of the first inflatable medical balloons are generally in parallel with the longitudinal axis of the second inflatable medical balloon. In another example, each of the plurality of first inflatable medical balloons is arranged to contact the second inflatable medical balloon.

In one example, each first inflatable medical balloon is arranged to contact each of a pair of first inflatable medical balloons adjacent thereto. In another example, the medical balloon sensing assembly further comprises a plurality of pressure sensors, each of the plurality of pressure sensors juxtaposed with a respective input port of a respective one of the plurality of catheters.

In one example, the medical balloon sensing assembly further comprises a plurality of flow sensors, each of the plurality of flow sensors juxtaposed with a respective input port of a respective one of the plurality of catheters. In another example, the medical balloon sensing assembly further comprises: a reservoir containing a predetermined volume of inflation fluid; and a plurality of pumps in fluid communication with the reservoir and the plurality of catheters, wherein each of the plurality of pumps is arranged to generate flow of the inflation fluid into a respective one of the plurality of first inflatable medical balloons via the respective one of the plurality of catheters.

In one further example, the medical balloon sensing assembly further comprises a plurality of sensors, each of the plurality of sensors juxtaposed with a respective one of the plurality of first inflatable medical balloons or with a respective one of the plurality of catheters, wherein, responsive to the output of each of the plurality of sensors, the respective one of the plurality of pumps is arranged to adjust the flow of the inflation fluid in relation to the respective one of the plurality of first inflatable medical balloons.

In one independent example, a medical balloon sensing assembly is provided, the medical balloon sensing assembly comprising: a handle; a catheter extending distally from the handle; an inflatable medical balloon secured by the catheter; one or more sensors juxtaposed with the inflatable medical balloon; and a sensor data unit in communication with the one or more sensors, wherein, responsive to an output of each of the one or more sensors, the sensor data unit is arranged to: determine a surface topography of a plurality of predetermined locations juxtaposed with the one or more sensors; determine an indication of a physical property of material of the plurality of predetermined locations; generate a 4-dimensional (4D) map of the plurality of predetermined locations, the 4D map comprising the determined surface topography and the determined indication of the physical property of the material; and output the generated 4D map.

In one example, the medical balloon sensing assembly further comprises a plurality of imaging markers disposed on the inflatable medical balloon, the one or more sensors comprising an imager arranged to image the imaging markers, wherein the determination of the surface topography is responsive to an output of the imager. In another example, the medical balloon sensing assembly further comprises a plurality of imaging markers disposed on the inflatable medical balloon, the one or more sensors comprising an optical camera arranged to image the imaging markers, wherein the determination of the surface topography is responsive to an output of the optical camera.

In one example the medical balloon sensing assembly further comprises a plurality of imaging markers disposed on the inflatable medical balloon, the one or more sensors comprising an ultrasound transducer arranged to image the imaging markers, wherein the determination of the surface topography is responsive to an output of the ultrasound transducer. In another example, the medical balloon sensing assembly further comprises a plurality of imaging markers disposed on the inflatable medical balloon, the one or more sensors comprising a thermal camera arranged to image the imaging markers, wherein the determination of the surface topography is responsive to an output of the thermal camera. In one further example, the indication of a physical property comprises an indication of a rate of thermal dispersion, the indication of the rate of thermal dispersion determined responsive to an output of the thermal camera.

In one example, the indication of a physical property comprises an indication of a rate of thermal dispersion, and wherein the plurality of sensors comprises a plurality of thermal sensors, the indication of the rate of thermal dispersion determined responsive to respective outputs of the plurality of thermal sensors. In another example, the indication of a physical property comprises a density, and wherein the plurality of sensors comprises an ultrasound transducer, the indication of the density determined responsive to an output of the ultrasound transducer.

In one example, the medical balloon sensing assembly further comprises a plurality of imaging markers disposed on the inflatable medical balloon, the one or more sensors comprising an ultrasound transducer arranged to image the imaging markers, wherein the determination of the surface topography is responsive to an output of the ultrasound transducer, and wherein the indication of a physical property comprises a density the indication of the density determined responsive to an output of the ultrasound transducer. In another example, the indication of a physical property comprises an extent of calcification.

In another independent example, an expandable structure sensing method is provided, the method comprising: delivering an expandable structure and a catheter to a predetermined anatomical location, the expandable structure secured to the catheter; inflating the delivered expandable structure; determining a surface topography of a plurality of predetermined locations juxtaposed with the expandable structure; determining an indication of a physical property of material of the plurality of predetermined locations; generating a 4-dimensional (4D) map of the plurality of predetermined locations, the 4D map comprising the determined surface topography and the determined indication of the physical property of the material; and outputting the generated 4D map.

In one example, the method further comprises imaging a plurality of imaging markers disposed on the expandable structure, wherein the determination of the surface topography is responsive to the imaging. In another example, the indication of a physical property comprises an indication of a rate of thermal dispersion.

In one example, the indication of a physical property comprises a density. In another example, the indication of a physical property comprises an extent of calcification.

Additional features and advantages of the invention will become apparent from the following drawings and description.

Unless otherwise defined, all technical and scientific terms used herein have the same meaning as commonly understood by one of ordinary skill in the art to which this invention pertains. In case of conflict, the patent specification, including definitions, governs. As used herein, the articles “a” and “an” mean “at least one” or “one or more” unless the context clearly dictates otherwise. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of examples of the instant inventive concepts. This is done merely for convenience and to give a general sense of the inventive concepts, and “a” and “an” are intended to include one or at least one and the singular also includes the plural unless it is obvious that it is meant otherwise.

The following examples and aspects thereof are described and illustrated in conjunction with systems, tools and methods which are meant to be exemplary and illustrative, but not limiting in scope. In various examples, one or more of the above-described problems have been reduced or eliminated, while other examples are directed to other advantages or improvements.

### BRIEF DESCRIPTION OF DRAWINGS

For a better understanding of the invention and to show how the same may be carried into effect, reference will now be made, purely by way of example, to the accompanying drawings in which like numerals designate corresponding sections or elements throughout.

With specific reference now to the drawings in detail, it is stressed that the particulars shown are by way of example and for purposes of illustrative discussion of the preferred examples of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice. In the accompanying drawings:

FIGS. 1A-1H illustrate various high-level views of a medical balloon sensing assembly comprising an inflatable medical balloon and a first example of a sensor position member, in accordance with certain examples;

FIGS. 1I-1JI illustrate various deployment states of the medical balloon sensing assembly of FIGS. 1A-1H, in accordance with certain examples;

FIGS. 1K-1L illustrate high level views of portions of the sensor position member of FIGS. 1A-1J exhibiting various depressions, in accordance with certain examples;

FIG. 2 illustrates a high-level perspective view of a medical balloon sensing assembly comprising an inflatable medical balloon and a second example of a sensor position member, in accordance with certain examples;

FIGS. 3A-3B illustrate various high-level perspective view of an inflatable medical balloon with embedded sensors, in accordance with certain examples;

FIGS. 3C-3D illustrate various high-level flow charts of methods of constructing the inflatable medical balloon of FIGS. 3A-3B, in accordance with certain examples;

FIGS. 4A-4B illustrate various deployment states of a medical balloon sensing assembly comprising an inflatable medical balloon and a prosthetic valve, in accordance with certain examples;

FIG. 4C illustrates a perspective view of the medical balloon sensing assembly of FIGS. 4A-4B, without the prosthetic valve;

FIG. 4D illustrates a cut away view of the medical balloon sensing assembly of FIGS. 4A-4B;

FIG. 4E illustrates a high-level perspective view of the prosthetic valve of FIGS. 4A-4B, in accordance with certain examples;

FIG. 5A illustrates a high-level perspective view of a medical balloon sensing assembly comprising an inflatable medical balloon and an ultrasound sensor, in accordance with certain examples;

FIG. 5B illustrates a high-level side view of a portion of the medical balloon sensing assembly of FIG. 5A, in accordance with certain examples;

FIG. 6 illustrates a high-level perspective view of a medical balloon sensing assembly comprising an inflatable medical balloon and a pump, in accordance with certain examples;

FIG. 7A illustrates a high-level side view of a medical balloon sensing assembly comprising an inflatable medical balloon and a thermal sensor in a first configuration, in accordance with certain examples;

FIG. 7B illustrates a high-level side view of a medical balloon sensing assembly comprising an inflatable medical balloon and a thermal sensor in a second configuration, in accordance with certain examples;

FIGS. 8A-8B illustrate various high-level views of a medical balloon sensing assembly comprising an inflatable medical balloon and a movable thermal camera, in accordance with certain examples;

FIGS. 9A-9B illustrate high-level side views of various configurations of a medical balloon sensing assembly comprising an inflatable medical balloon and a thermal sensor secured to a guidewire, in accordance with certain examples;

FIGS. 10A-10C illustrate various high-level views of an imaging system, and an associated medical balloon sensing assembly, in accordance with certain examples;

FIGS. 11A-11C illustrate various high-level views of a first configuration of a medical balloon sensing assembly comprising a plurality of inflatable medical balloons, in accordance with certain examples;

FIGS. 11D-11E illustrate various high-level views of a first configuration of a medical balloon sensing assembly comprising a plurality of inflatable medical balloons, in accordance with certain examples;

FIGS. 12A-12F illustrate high-level flow charts of various medical balloon sensing methods, in accordance with certain examples.

### DETAILED DESCRIPTION OF CERTAIN EXAMPLES

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout. In order to avoid undue clutter from having too many reference numbers and lead lines on a particular drawing, some components will be introduced via one or more drawings and not explicitly identified in every subsequent drawing that contains that component.

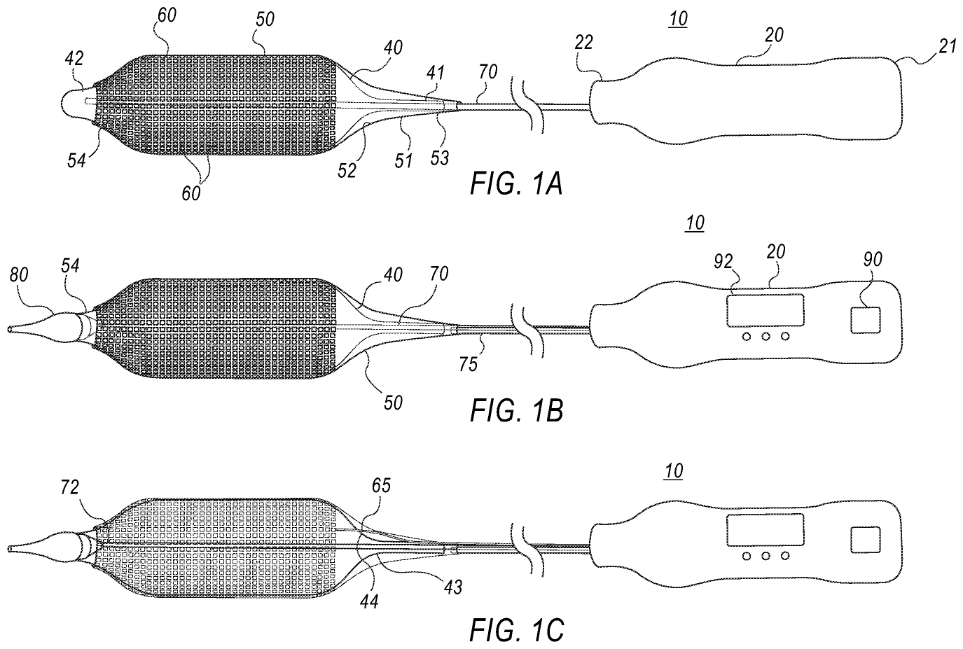

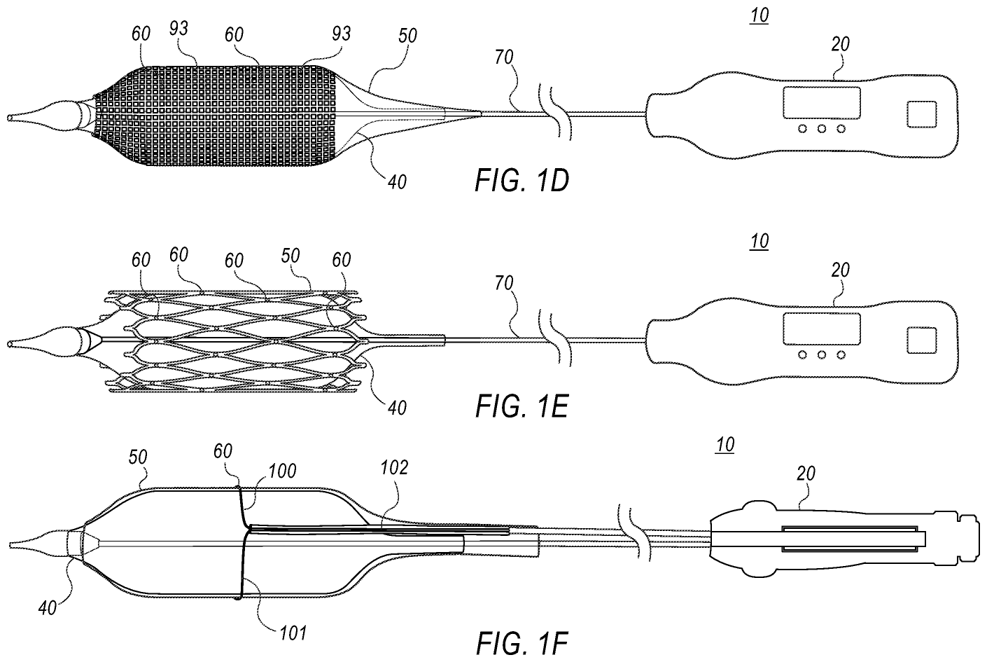

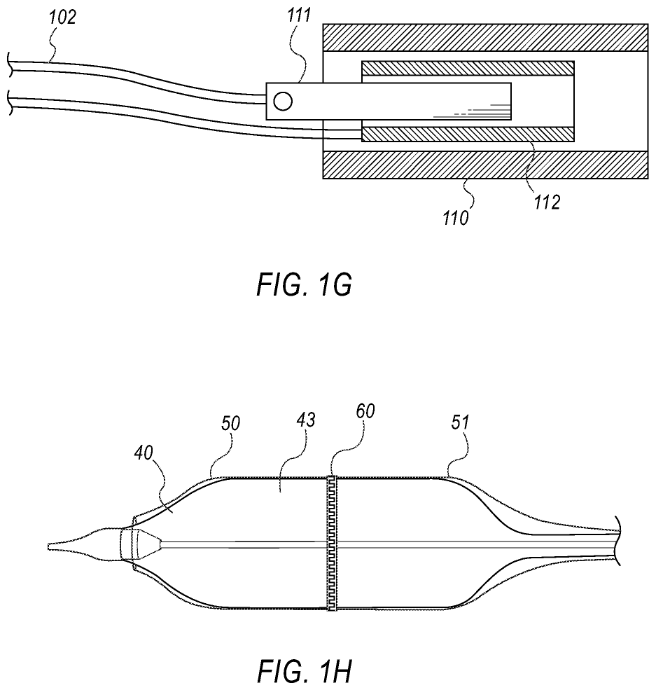

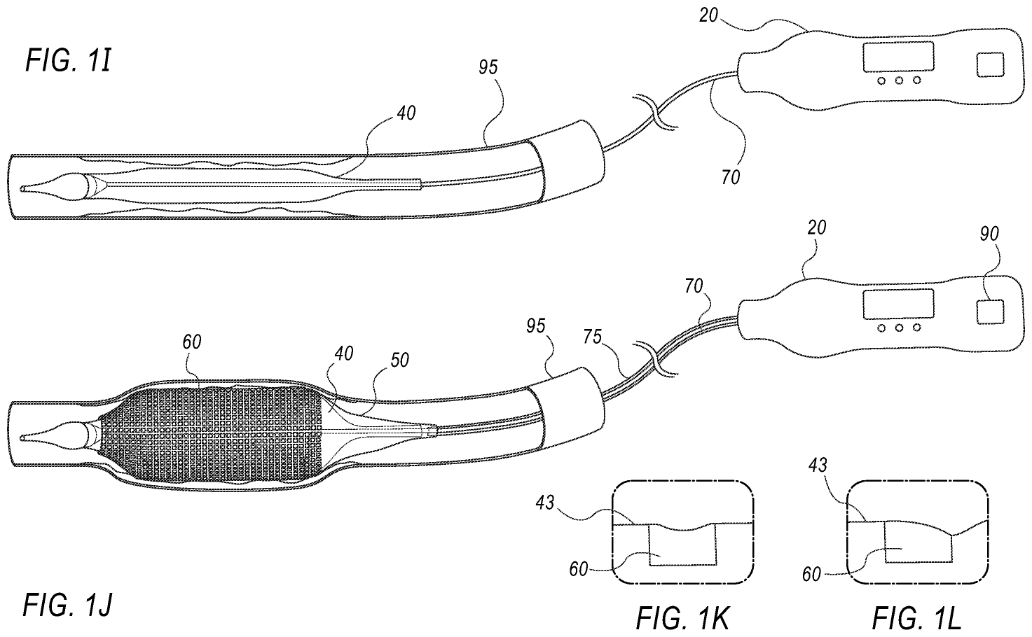

FIGS. 1A-1D illustrate a high-level perspective view of a medical balloon sensing assembly 10, in accordance with certain examples. Medical balloon sensing assembly 10 comprises: a handle 20, exhibiting a proximal end 21 and a distal end 22; an inflatable medical balloon 40, extending from a proximal end 41 to a distal end 42, and exhibiting an outer face 43 and an inner face 44 (illustrated in FIG. 1C) opposing outer face 43; a sensor position member 50 exhibiting an outer face 51 and an inner face 52 opposing outer face 51; one or more sensors 60; one or more optional communication mediums 65 (illustrated in FIG. 1C); a catheter 70 (illustrated in FIG. 1B) extending towards a distal end 72; and an optional catheter 75. In one example, sensor position member 50 further exhibits a proximal section 53 and a distal section 54. FIG. 1B illustrates a more detailed example of medical balloon sensing assembly 10, further optionally comprising: a nosecone 80; a sensor data unit 90; and one or more visual or auditory informative elements configured to provide visual or auditory information and/or feedback to a user or operator of medical balloon sensing assembly 10, such as a display 92, LED lights, speakers (not shown) and the like. FIG. 1C illustrates a partially transparent view of the detailed example of medical balloon sensing assembly 10. FIG. 1D illustrates medical balloon sensing assembly 10 without optional catheter 75.

In one example, as illustrated in FIG. 1D, medical balloon sensing assembly further comprises a plurality of heating elements 93. Each heating element is secured in relation to a respective predetermined point on outer surface 43 or inner surface 44 of inflatable medical balloon 40. Each heating element 93 is arranged to generate heat at a predetermined temperature. In one further example, each heating element 93 comprises a conductive material arranged to generate heat responsive to an electrical current flowing therethrough. In another further example, each heating element 93 comprises a thermistor.

The term “proximal”, as used herein, generally refers to the side or end of any device or a component of a device, which is closer to handle 20 or an operator of handle 20 when in use. The term “distal”, as used herein, generally refers to the side or end of any device or a component of a device, which is farther from handle 20 or an operator of handle 20 when in use.

Although FIGS. 1A-1D are described herein together, this is not meant to be limiting in any way, and medical balloon sensing assembly 10 can be provided without optional catheter 75, nosecone 80 and sensor data unit 90, without exceeding the scope of the disclosure. FIGS. 1A-1D illustrate inflatable medical balloon 40 in an inflated state, as will be described below.

In one example, inflatable medical balloon 40 is formed of polyester, nylon, polyether block amide, polyurethane and/or silicone. Although inflatable medical balloon 40 is illustrated as being generally cylindrical shaped, this is not meant to be limiting in any way. In another example (not shown), inflatable medical balloon 40 can be shaped as a cone, an ellipse, a sphere, or any other suitable shape known to those skilled in the art. In one example, inflatable medical balloon 40 is transparent, semi-transparent or opaque.

In one example, sensor position member 50 at least partially circumferentially surrounds outer face 43 of inflatable medical balloon 40. The term “circumferentially surrounds”, as used herein, means to extend along the circumference thereof. In one further example, sensor position member 50 is a closed sleeve, optionally shaped similarly to inflatable medical balloon 40. In one example, distal section 54 of sensor position member 50 extends over inflatable medical balloon 40 and proximal section 53 extends over catheter 75 and/or catheter 70 towards handle 20. In another example (not shown), sensor position member 50 is shaped as an open sleeve.

In another example, as shown in FIG. 1E, sensor position member 50 comprises a collapsible stent and sensors 60 are disposed on the struts of the collapsible stent. Particularly, when inflatable medical balloon 40 is in a collapsed state the stent is also in a collapsed state. Similarly, when inflatable medical balloon is in an inflated state the stent is expanded. In one further example, the stent is expanded by the inflation of inflatable medical balloon 40, as known to those skilled in the art in relation to balloon expandable stents.

In one example, the collapsible stent is removable from the patient lumen when inflatable medical balloon 40 is deflated and removed from the patient lumen. In one further example (not shown), a collapsing mechanism is provided on the collapsible stent, such as a lasso-shaped mechanism, which collapses the stent when inflatable medical balloon 40 is deflated. Thus, the collapsible stent is removed from the patient lumen when inflatable medical balloon 40 is removed. In another further example (not shown), the collapsible stent is self-expanding such that each portion of the collapsible stent expands as it is released from a delivery sheath, and a portion of the collapsible stent remains inside the delivery sheath. In such an example, a collapsing mechanism is provided inside the delivery sheath to gradually collapse the stent from inside the delivery sheath. In another further example, the collapsible stent is constructed such that the collapsed state is a resting state thereof. In such an example, when inflatable medical balloon 40 is inflated, the stent is expanded and inflatable medical balloon 40 keeps the elastic forces of the stent from returning the structure to its collapsed resting state. Thus, when inflatable medical balloon 40 is deflated, the stent collapses back to its resting state and can then be removed from the patient lumen.

Although sensor position member 50 is illustrated as circumferentially surrounding the outer face 43 around the entire circumference thereof, this is not meant to be limiting in any way and sensor position member 50 can be arranged to circumferentially surround any portion of the circumference of outer face 43 of inflatable medical balloon 40 without exceeding the scope of the disclosure. Similarly, although sensor position member 50 is illustrated as circumferentially surrounding the outer face 43 along nearly the entirety of the length thereof, this is not meant to be limiting in any way and sensor position member 50 can be arranged to extend along any portion of the length of outer face 43 without exceeding the scope of the disclosure. Although a single sensor position member 50 is illustrated, this is not meant to be limiting in any way. In another example (not shown), a plurality of sensor position members 50 are provided, each at least partially circumferentially surrounding outer face 43 of inflatable medical balloon 40.

In one example, sensor position member 50 is generally formed of the same material as inflatable medical balloon 40. In another example, sensor position member 50 comprises a woven and/or braided material. In another example, sensor position member 50 exhibits generally the same elasticity and tearing strength as inflatable medical balloon 40. In one further example, distal section 54 exhibits generally the same elasticity and tearing strength as inflatable medical balloon 40.

In one example, at least one sensor 60 comprises at least one force sensor. The term “force sensor”, as used herein, means any sensor that senses the magnitude of a force, or pressure, applied thereto. In one further example, each sensor 60 comprises: a piezoresistive sensor, such as a strain gauge or a strain gauge bridge, the resistance of the piezoresistive sensor being a respective predetermined function of the force applied thereto; a piezoelectric sensor, the voltage at the output of the piezoelectric sensor being a respective predetermined function of the force applied thereto; a capacitive sensor, the capacitance of the capacitive sensor being a respective predetermined function of the force applied thereto; and/or an optical sensor, the optical interferometry of the optical sensor being a respective predetermined function of the force applied thereto. In another further example, each sensor 60 comprises a wide diameter pressure sensor.

In another example, at least one sensor 60 comprises at least one flex sensor. Each flex sensor 60 is arranged to measure how much it is bent. The terms bend and flex, as used herein, are interchangeable.

In another example, at least one sensor 60 comprises at least one diameter sensor. In one further example, as illustrated in FIGS. 1F-1G, sensor 60 comprises a radially translatable member 100 and a linear displacement sensor 110. In one example, radially translatable member 100 comprises a loop shaped balloon portion 101 and a connection portion 102, connection portion coupled to an input of linear displacement sensor 110, as will be described below. In another example, linear displacement sensor 110 is implemented as a linear variable differential transformer (LVDT) sensor, comprising a transformer core 111 within a tube 112.

In another further example, at least one sensor 60 comprises one or more distance sensors arranged to detect a distance from the respective sensor 60 to a predetermined point. In one example, the one or more distance sensors 60 comprise: one or more optical sensors arranged to detect the dispersion and/or absorption of light; and/or one more ultrasound sensor. In another example, at least one sensor 60 comprises one or more force sensors and one or more diameter sensors. Although sensors 60 are illustrated in FIGS. 1A-1E as being square shaped, this is not meant to be limiting in any way, and sensors 60 can be any suitable shape, without exceeding the scope of the disclosure.

In another example, at least one sensor 60 comprises one or more thermal sensors. In one further example, the one or more thermal sensors 60 comprise a thermal camera. In one further example, the thermal camera comprises an infrared camera, such as a thermographic camera. In another further example, one or more thermal sensors 60 each comprise a respective thermocouple. In another further example, one or more thermal sensors 60 each comprise a respective thermistor. In one example, the one or more thermal sensors 60 comprises a plurality of thermal sensors 60, each thermal sensor 60 arranged to alternately operate in a heating mode and a sensing mode. In the heating mode, the respective thermal sensor 60 is arranged to generate heat at a predetermined temperature. In the sensing mode, the respective thermal sensor 60 is arranged to sense the temperature thereat. In one example, the heating mode is responsive to a current exhibiting a first magnitude being applied thereto and the sensing mode is responsive to a current exhibiting a second magnitude being applied thereto, the first magnitude being greater than the second magnitude. For example, in an example where each thermal sensor 60 comprises a thermistor, a current exhibiting a large magnitude is driven through the respective thermal sensor 60.

In another example, at least one sensor 60 comprises one or more electrical conductance sensors 60. Particularly, an output of each electrical conductance sensor 60 indicates the electrical conductance of the material juxtaposed therewith. In one example, each electrical conductance sensor 60 comprises a pair of electrical leads and a current magnitude sensor. In such an example, a predetermined voltage is optionally applied to the pair of electrical leads and the magnitude of current flowing between the electrical leads is measured by the current magnitude sensor. Responsive to the measured current magnitude and the predetermined voltage, the electrical resistance of the material is determined. In one example, the measurement of the electrical conductance is performed by sensor data unit 90, responsive to a predetermined current and/or voltage provided by sensor data unit 90 to the respective electrical conductance sensor 60, as described below. In another example, each electrical conductance sensor 60 comprises dedicated circuitry that measures electrical conductance of the material juxtaposed with the respective electrical conductance sensor 60, and sensor data unit 90 reads the output of the respective electrical conductance sensor 60, as described below.

In one example, each communication medium 65 is configured to allow: electrical communication via a conductive material, such as a wire; and/or optical communication, e.g. via an optical fiber.

In one example, a series of traces form interconnects (e.g., serpentine interconnects) between sensors 60 and/or between sensors 60 and an actuator or other centralized control element. Non-limiting example approaches for arranging sensors and interconnects on a deformable surface are described in Krishnan et al., “Epidermal electronics for noninvasive, wireless, quantitative assessment of ventricular shunt function in patients with hydrocephalus,” Science Translational Medicine 10, eeat8437 (2018), the entire contents of which are incorporated herein by reference.

In one example, sensor data unit 90 comprises a central processing unit (CPU), a microprocessor, a microcomputer, a programmable logic controller, an application-specific integrated circuit (ASIC) and/or a field-programmable gate array (FPGA), without limitation. In another example, sensor data unit 90 comprises electrical and/or electro-optical circuitry.

Inflatable medical balloon 40 is secured by catheter 70 and catheter 70 extends into inflatable medical balloon 40, as known to those skilled in the art of balloon catheters. In another example, nosecone 80 is secured to distal end 72 of catheter 70 and/or to distal end 42 of inflatable medical balloon 40.

Sensor position member 50 is juxtaposed with outer face 43 of inflatable medical balloon 40. In an example where sensor position member 50 comprises a sleeve, sensor position member 50 covers at least a portion of inflatable medical balloon 40 such that inner face 52 of sensor position member 50 is juxtaposed with outer face 43 of inflatable medical balloon 40. In one example, as illustrated in FIGS. 1B-1C, sensor position member 50 is secured to optional catheter 75. In one further example, optional catheter 75 is positioned alongside catheter 70. In another further example (not shown), optional catheter 75 is disposed within catheter 70. In another example, as illustrated in FIG. 1D, sensor position member 50 is secured to catheter 70 and optional catheter 75 is not provided. In another example (not shown), sensor position member 50 is attached to inflatable medical balloon 40. In one example, catheter 70 extends from handle 20. In another example, optional catheter 75 extends from handle 20.

At least one sensor 60 is secured to sensor position member 50. In one example, at least one sensor 60 is secured to sensor position member 50 by an adhesive and/or a securing member, such as one or more sutures (not shown). In one example, at least one sensor 60 is positioned on outer face 51 of sensor position member 50 and/or inner face 52 of sensor position member 50. In another example, at least one sensor 60 is embedded within the material of sensor position member 50. In another example, where sensor position member 50 comprises a woven or braided material, at least one sensor 60 is woven within the material of sensor position member 50.

In one example, a plurality of sensors 60 are provided, sensors 60 positioned on sensor position member 50 at predefined positions and exhibiting respective predetermined distances between adjacent sensors 60. In one further example, sensors 60 are positioned on sensor position member 50 in a grid pattern. In another further example, the predetermined distances between adjacent sensors 60 are substantially equal to each other. In another further example, the predetermined distances between adjacent sensors 60 are each shorter than a width of each sensor 60. In another further example, sensors 60 are positioned on a sensor area of sensor position member 50 such that the sensor area is essentially completely covered by sensors 60. In another further example, one or more sensors 60 are positioned on sensor position member 50, juxtaposed with distal end 42 of inflatable medical balloon 40. Thus, each sensor 60 is juxtaposed with inflatable medical balloon 40.

In one example, where sensor 60 comprises radially translatable member 100 and a linear displacement sensor 110, loop shaped balloon portion 101 of radially translatable member 100 is secured to sensor position member 50 and surrounds outer face 43 of inflatable medical balloon 40 and outer face 51 of sensor position member 50. As described above, in one example, balloon portion 101 is secured to outer face 51 of sensor position member 50, inner face 52 of sensor position member 50 and/or embedded within sensor position member 50. Connection portion 102 extends from balloon portion 101 and is coupled to the input of linear displacement sensor 110. An output of linear displacement sensor is in communication with sensor data unit 90.

In one example, where linear displacement sensor 110 is implemented as an LVDT sensor, connection portion 102 is coupled to core 111. Although the coupling of connection portion 102 to core 111 is illustrated as being a direct connection, this is not meant to be limiting in any way. In another example, a cable (not shown) extends from core 111 and connection portion 102 is coupled to the cable. Although balloon portion 101 is illustrated as being loop shaped and completely surrounding outer face 43 of inflatable medical balloon 40, this is not meant to be limiting in any way. In another example (not shown), balloon portion 101 circumferentially surrounds a portion of outer face 43. In another example (not shown), balloon portion 101 comprises a first section extending in a first circumferential direction and a second section extending in a second circumferential direction, the second circumferential direction opposing the first circumferential direction.

In one example (not shown), where at least one sensor 60 comprises at least one distance sensor, each sensor 60 comprises a transmitter and a receiver, each positioned at a respective position on sensor position member 50 such that the transceiver and receiver oppose each other, thereby allowing measurement of the distance between the transmitter and the receiver. In another example, where at least one sensor 60 comprises at least one distance sensor, each sensor 60 is a standalone sensor positioned at a respective position on sensor position member 50. In such an example, each sensor 60 measures the distance to a point, and/or area of position member 50 and/or inflatable medical balloon 40 opposing the position of the respective sensor 60.

In one example, each sensor 60 is in communication with sensor data unit 90. In one further example, each sensor 60 is in communication with sensor data unit 90 via a respective optional communication medium 65. Only one communication medium 65 is illustrated for clarity, however this is not meant to be limiting in any way. In another further example, each sensor 60 is in wireless communication with sensor data unit 90. In one example, sensors 60 are separated into a plurality of sets, each set of sensors 60 in communication with sensor data unit 90 via a respective communication medium 65. In such an example, as will be described below, the operation of sensors 60 is multiplexed such that only one respective sensor 60 of each set outputs data each time.

In one example, sensors 60 include one or more thin wires, where one or more sensors form one or more respective portions of the wire(s), and where the wire(s) are connected to a ground. In such an example, the wire(s) optionally come out of catheter 70 and/or optional catheter 75, surround inflatable medical balloon 40, then go back down catheter 70 (e.g., multiplexing energy all around the loop), where at least some of the sensors/wires 60 are connected to a common ground in medical balloon sensing assembly 10. In one further example, the wire(s) and/or other sensor assembly forms a net that is coupled to inflatable medical balloon 40. In another example, sensors 60 are mounted on a respective catheter or mounted on a wire then coiled around inflatable medical balloon 40.

In one example, each sensor 60 is operated by sensor data unit 90 such that the sensing of sensor 60 is performed in cooperation with sensor data unit 90. For example, in an example where each sensor 60 comprises a strain gauge bridge, sensor data unit 90 applies a predetermined excitation voltage at the input leads of the bridge and measures the voltage at the output leads of the bridge. Sensor data unit 90 then determines the applied force, or pressure, from the measured output voltage. In another example, each sensor 60 comprises dedicated circuitry for operation and sensor data unit 90 receives the measured data from the sensors 60. In another example (not shown), each sensor 60 is in wireless communication with an external computing device.

In one example, as illustrated in FIG. 1H, one or more sensors 60 comprise a strain gauge circumferentially surrounding outer face 51 of sensor position member 50 and outer face 43 of inflatable medical balloon 40. For simplicity, handle 20 is not illustrated in FIG. 1H. Although FIGS. 1F-1H are illustrated in an example where a single sensor 60 is provided (radially translatable member 100 and linear displacement sensor 110 of FIGS. 1F-1G or strain gauge 60 of FIG. 1H, this is not meant to be limiting in any way. In another example, a plurality of such sensors 60 are provided. In another example, the respective illustrated single sensor 60 is provided along with additional sensors 60 of one or more different types.

FIGS. 1I-1J illustrate various deployment stages of medical balloon sensing assembly 10 into a patient lumen 95, in accordance with certain examples, FIGS. 1A-1J being described together. Patient lumen 95 is shown in a cut away view. In operation, a user holds handle 20 and maneuvers catheter 70 such that inflatable medical balloon 40 is inserted into patient lumen 95 and positioned at a desired implantation site. In one example, the desired implantation site is an annulus of the heart. As illustrated in FIG. 1I, inflatable medical balloon 40 is initially in a deflated state. In one example, inflatable medical balloon 40 is inserted into patient lumen 95 in order to measure the diameter of patient lumen 95 and/or identify calcifications within patient lumen 95 for preparation of implantation of a prosthetic heart valve. For clarity, FIG. 1I is illustrated without sensor position member 50 and optional catheter 75.

In one example (not shown), a guidewire extends through a central lumen of catheter 70 and an inner lumen of nosecone 80, so that catheter 70 can be advanced over the guidewire through the patient's vasculature. In one example, during delivery, handle 20 is maneuvered by an operator (e.g., a clinician or a surgeon) to axially advance or retract catheter 70 and/or catheter 75 through the patient's vasculature. In another example, handle 20 comprises one or more operating interfaces, such as steerable or rotatable adjustment knobs, levers, sliders, buttons (not shown) and other actuating mechanisms, which are operatively connected to catheter 70 and/or catheter 75 and configured to produce axial movement of catheter 70 and/or catheter 75 in the proximal and distal directions.

After positioning inflatable medical balloon 40 at the desired implantation site, an inflation fluid (not shown) is injected into inflatable medical balloon 40. In one example, the inflation fluid comprises saline. In another example, the inflation fluid comprises sterile water. Although the above examples relate to liquid fluids, this is not meant to be limiting in any way. In one example, the inflation fluid is injected using a syringe or a manual pump, such that inflation fluid is transferred from a reservoir to inflatable medical balloon 40. In another example, as will be described below, the inflation fluid is injected using an electrically powered pump controlled by a computerized controller. In one example, the inflation fluid enters inflatable medical balloon 40 via openings in catheter 70 (not shown). In one example, the temperature of the interior of inflatable medical balloon 40, i.e. the cavity formed by inner face 44, is less than 31 degrees Celsius, optionally less than 21 degrees Celsius, optionally less than 10 degrees Celsius, optionally less than 4 degrees Celsius. In one further example, this is achieved by providing inflation fluid exhibiting a temperature less than 31 degrees Celsius, optionally less than 21 degrees Celsius optionally less than 10 degrees Celsius, optionally less than 4 degrees Celsius.

As the inflation fluid fills inflatable medical balloon 40, inflatable medical balloon 40 is inflated towards the walls of patient lumen 95, as illustrated in FIG. 1J. As described above, sensor position member 50 is juxtaposed with outer face 43 of inflatable medical balloon 40, such that the inflation of inflatable medical balloon 40 causes sensor position member 50 to expand as well. In an example where at least one sensor 60 comprises a diameter sensor, sensor data unit 90 determines a diameter indication of inflatable medical balloon 40 and in one further example outputs the diameter indication. In one example, the diameter indication of inflatable medical balloon 40 is determined at predetermined intervals during inflation. In another example, the diameter indication of inflatable medical balloon 40 is determined continuously during inflation.

The term “diameter indication”, as used herein, means a predetermined indication of a diameter. In one example, the diameter indication comprises the radial diameter of inflatable medical balloon 40. In another example, the diameter indication comprises a predetermined function of the diameter. In another example, the diameter indication comprises the value of a change in the diameter. Particularly, in such an example, sensor data unit 90 identifies the rate of change in the diameter of inflatable medical balloon 40 and/or the amount the diameter has changed between two or more measurements. In one further example, upon detection that the diameter is no longer increasing, i.e. outer face 43 of inflatable medical balloon 40 is pushing against the walls of patient lumen 95, sensor data unit 90 outputs a signal indicating that the diameter is no longer increasing. In such a case, it may be desired to cease the injection of inflation fluid into inflatable medical balloon 40, so as to reduce risk of damaging the surrounding tissue due to increased pressure applied by the balloon thereto. In one example, sensor data unit 90 is further arranged to control a flow controller (not shown) to prevent the flow of any more inflation fluid into inflatable medical balloon 40.

In one example, responsive to the determined diameter indication, sensor data unit 90 outputs an indication of the maximum diameter allowed for expansion in patient lumen 95. For example, inflatable medical balloon 40 can be used to determine the size of patient lumen 95 for purposes of determining the target expansion diameter of a prosthetic valve. Thus, sensor data unit 90 measures the diameter of patient lumen 95 when inflatable medical balloon 40 is inflated, thereby identifying the appropriate expansion diameter for a prosthetic valve.

As described above, in one example sensor 60 comprises a radially translatable member 100 and a linear displacement sensor 110. In such an example, balloon portion 101 of radially translatable member 100 is radially translated outwards as the perimeter of the balloon 40 is increased, thereby linearly pulling connection portion 102. The linear translation of connection portion 102 causes relative movement between core 111 and tube 112 of linear displacement sensor 110. This relative movement is a predetermined function of the amount of radial translation of balloon portion 101 and generates a respective voltage. Sensor data unit 90 measures the voltage at the output of linear displacement sensor 100 and responsive thereto determines a diameter indication of inflatable medical balloon 40, optionally the diameter indication being the diameter thereof.

In one example, where at least one sensor 60 comprises one or more flex sensors, sensor data unit 90 determines a diameter indication of inflatable medical balloon 40 responsive to an output of flex sensor/s 60. Particularly, each flex sensor 60 is positioned such that the flex thereof decreases as inflatable medical balloon 40 is inflated. In one example, sensor data unit 90 compares the amount of flex of each flex sensor 60 with the amount of flex of the respective flex sensor 60 when inflatable medical balloon 40 was in the initial deflated state. In another example, sensor data unit 90 identifies the rate of change in the flex of the flex sensor/s 60. In one further example, as described above, upon detection that the diameter is no longer increasing, sensor data unit 90 outputs a signal indicating that the diameter is no longer increasing.

In one example, sensor data unit 90 determines a recoil of inflatable medical balloon 40 and the diameter indication comprises an indication of the determined recoil. Particularly, in one example, as will be described below, a prosthetic valve is provided over inflatable medical balloon 40 and expanded by inflatable medical balloon 40. When inflatable medical balloon 40 ceases to be inflated, the prosthetic valve ceases its expansion. However, due to the forces applied between the prosthetic valve and the patient lumen, there is a small recoil of the prosthetic valve, i.e. the diameter of the prosthetic valve is slightly reduced. As a result, the diameter of inflatable medical balloon 4 is also slightly reduced. Thus, in such an example, sensor data unit 90 determines the diameter of inflatable medical balloon before and after the recoil. The difference in diameter of inflatable medical balloon 40 from before the recoil and after the recoil, i.e. the recoil indication, provides an indication of the rigidity of the tissue surrounding the patient lumen. Therefore, an indication of the extent of calcification of the patient lumen is provided.

As inflatable medical balloon 40 presses against the walls of patient lumen 95, a force is applied to sensor position member 50. In an example where sensor/s 60 comprises at least one force sensor, the at least one force sensor 60 measures the applied force and/or pressure. Responsive to the received measurements of sensor/s 60, sensor data unit 90 determines the pressure between inflatable medical balloon 40 and the walls of patient lumen 95. In one example, where one or more sensors 60 are juxtaposed with distal end 42 of inflatable medical balloon 40, i.e. reference sensors, sensor data unit 90 determines the pressure against patient lumen 95 by subtracting the pressure values measured by the reference sensors from the pressure values measured by the rest of sensors 60. Particularly, inflatable medical balloon 40 may not inflate uniformly and there can be deformation of the balloon during inflation. This deformation can generate pressure against sensors 60, however this pressure is not applied to the walls of patient lumen 95. By subtracting the pressure of the reference sensors, a more accurate measurement of the pressure applied to the walls of patient lumen can be achieved.

In another example, one or more displacement sensors 60 are juxtaposed with distal end 42 of inflatable medical balloon 40. In such an example, these displacement sensors 60 measure the axial elongation of inflatable medical balloon 40. This axial elongation affects the pressure measurements, as described above. Therefore, in one example sensor data unit 90 determines a predetermined function of the received pressure measurements and the displacement measurements to determine an accurate pressure measurement that is not affected by the axial elongation. In one example, the ratio of the pressure that is applied to axial elongation of inflatable medical balloon 40 to the pressure that is applied to radial expansion of inflatable medical balloon 40 should be 0.5. Thus, if the ratio is greater than 0.5, it is an indication that inflatable medical balloon 40 is fully expanded, or close to fully expanded. In another example, alternatively or additionally, sensor data unit 90 determines a predetermined function of the flow sensor measurements and the displacement measurements to determine an accurate flow measurement of the inflation fluid that is acting to radially inflate inflatable medical balloon 40 and does not include the inflation fluid that is acting to axially elongate inflatable medical balloon 40.

In another example, sensor data unit 90 determines an average, or other predetermined function, of the measurements of sensors 60 in order to achieve a more accurate measurement of the pressure between inflatable medical balloon 40 and the walls of patient lumen 95. In an example where at least one sensor 60 comprises a wide diameter pressure sensor, an accurate measurement of the pressure between inflatable medical balloon 40 and the walls of patient lumen 95 is provided.

In another example, where a plurality of diameter sensors are provided, such as a plurality of radially translatable members 100 axially spaced from each other along inflatable medical balloon 40, sensor data unit 90 determines a diameter profile of inflatable medical balloon 40, which can exhibit a plurality of different measured diameters if inflatable medical balloon 40 expands non-uniformly with different diameters at different axial positions (e.g. a frustoconical profile of inflatable medical balloon 40). In one example, sensor data unit 90 determines the difference between the output of several of the diameter sensors, and the surface topography of patient lumen 95 is determined responsive to the difference between the outputs of the diameter sensors.

As described above, in one example the measurements of sensor/s 60 are performed in cooperation with sensor data unit 90. Alternatively, the measurements of sensor/s 60 are performed by dedicated circuitry of sensor/s 60 and transmitted to sensor data unit 90.

In one example, where at least one sensor 60 comprises a plurality of force sensors, such as a grid of force sensors, sensor data unit 90 determines a map of the forces applied to inflatable medical balloon 40. The term “map of forces”, as used herein, means a representation, graphical or otherwise, of the locations and magnitudes of forces and/or pressures applied to inflatable medical balloon 40. In one example, sensor data unit 90 outputs an indication of the determined map. In one further example, outputting the indication of the determined map comprises controlling display 92, or an external user display, to display the determined map.

In one example, sensor data unit 90 identifies any areas in the map of forces which exhibit increased forces applied thereto. These areas may be indicative of calcifications being pressed against inflatable medical balloon 40. In one further example, the increased forces are identified by comparing the forces to one or more predetermined threshold values. Alternatively, or additionally, the increased forces are identified in relation to an average, or other function, of the identified force values from all or a portion of sensors 60. In one example, the magnitude of force applied by a calcification is determined by subtracting from the measured force, applied by the calcification, an average of the forces measured by the rest of sensors 60.

In one example, responsive to an indication of an area exhibiting increased force or pressure, sensor data unit 90 outputs a signal indicative of the presence, location and/or applied magnitude of force or pressure of the one or more calcifications. In another example, responsive to the determined magnitude of force applied by the one or more calcifications, sensor data unit 90 determines a maximum inflation pressure value allowed for inflation of inflatable medical balloon 40 and outputs a respective signal indicating the determined maximum inflation pressure value. In one further example, sensor data unit 90 monitors the determined magnitude of pressure applied by the calcifications and the pressure applied by the uncalcified portions of patient lumen 95. The maximum inflation pressure value can then be determined by identifying the pressure applied by the uncalcified portions of patient lumen 95 when the pressure applied by one or more calcifications reaches a predetermined value.

In another example, responsive to the determined magnitude of force applied by the one or more calcification, sensor data unit 90 determines an appropriate orientation for the prosthetic heart valve. For example, in one example, sensor data unit 90 determines an appropriate orientation such that predetermined portions of the prosthetic heart valve won't come in contact with the identified calcification. In one example, sensor data unit 90 identifies the location of the calcification in relation to predefined locations on inflatable medical balloon 40, these predefined locations corresponding to the predetermined portions of the prosthetic heart valve. Sensor data unit 90 thus determines the appropriate orientation for the prosthetic heart valve in relation to the orientation of inflatable medical balloon 40. In one example, sensor data unit 90 outputs an indication of the determined orientation.

In another example, responsive to the determined magnitude of force applied by the one or more calcification, sensor data unit 90 determines whether implantation of a balloon expandable prosthetic valve at the anatomical location where inflatable medical balloon 40 is currently located is viable. Particularly, if it is not viable, a different type of prosthetic valve can be implanted, such as a mechanically expandable prosthetic valve. In one example, sensor data unit 90 outputs an indication of the determined viability. In one example, viability is determined by determining the maximum pressure values applied by one or more calcifications. If the determined maximum pressure exceeds a predetermined pressure threshold, implantation of the balloon expandable prosthetic valve is considered unviable.

In one example, where at least one sensor 60 comprises one or more flex sensors, sensor data unit 90 determines the amount each flex sensor 60 is bent, as described above. A calcification which protrudes into inflatable medical balloon 40 will create a depression in outer face 43. In the event that this depression is formed at the position of a flex sensor 60, the respective flex sensor 60 bends inwards, i.e. towards catheter 70, as illustrated in FIG. 1K. In one example, responsive to the output of the respective flex sensor 60, sensor data unit 90 identifies the inward bend and outputs a signal indicative of an identified calcification.

In one further example, responsive to the output of the respective flex sensor 60, sensor data unit 90 further determines the amount of inward flex experienced by the respective flex sensor 60 and compares the amount of inward flex to a respective predetermined threshold value, optionally 30 degrees. Responsive to an outcome of the comparison indicating that the amount of inward flex is greater than the predetermined threshold value, sensor data unit 90 outputs a signal indicating that the identified calcification is hazardous. In one example, the output signal further indicates the position of the identified calcification in relation to inflatable medical balloon 40 and/or patient lumen 95.

In the event that the depression formed by a calcification is near a flex sensor 60, the outwards flex of the respective flex sensor 60 will increase, as illustrated in FIG. 1L. In one example, responsive to the output of the respective flex sensor 60, sensor data unit 90 compares the amount of outwards flex of the respective flex sensor 60 to a respective predetermined threshold value, optionally, 30 degrees. In another example, responsive to the output of the respective flex sensor 60, sensor data unit 90 compares the amount of outwards flex of the respective flex sensor 60 to the amount of outwards flex of the other flex sensors 60. The difference between the flex amount values is compared to a respective predetermined threshold value. Responsive to an outcome of the comparison indicating that the amount of outward flex, or the flex amount difference, is greater than the respective predetermined threshold value, sensor data unit 90 outputs a signal indicating that the identified calcification is hazardous. In one example, the output signal further indicates the position of the identified calcification in relation to inflatable medical balloon 40 and/or patient lumen 95, as described above.

In one example, responsive to the respective outputs of flex sensors 60, sensor data unit 90 determines a height-diameter aspect ratio of each identified calcification. The term “height-diameter aspect ratio” means the ratio between the height of the calcification and a diameter thereof. Particularly, a lower aspect ratio is indicative of a wide and shallow calcification, which is less likely to be pushed into the tissue and/or to tear inflatable medical balloon 40 or certain portions of a prosthetic heart valve to be later implanted. In contrast, a higher aspect ratio is indicative of a thinner calcification, which is more likely to be pushed into the tissue and/or tear inflatable medical balloon 40 or the respective portions of the prosthetic heart valve.

In one example, responsive to an identification of a calcification, sensor data unit 90 compares the identified location of the calcification to predetermined portions of a prosthetic heart valve, as will be described below. In such an example, responsive to an outcome of the comparison, sensor data unit 90 determines an appropriate orientation for the prosthetic heart valve, as described above.

In another example, as described above, sensor data unit 90 determines a maximum pressure value for inflating inflatable medical balloon 40, i.e. the maximum pressure allowed between inflatable medical balloon 40 and the walls of patient lumen 95. In another example, sensor data unit 90 compares the number, locations and/or height-diameter aspect ratios of the identified one or more calcifications to predetermined threshold values. In one further example, a predetermined function of the number, locations and/or height-diameter aspect ratios of the identified one or more calcifications is compared to the predetermined threshold values. In one example, responsive to an outcome of the comparison, sensor data unit 90 determines the feasibility for implanting a balloon expandable prosthetic heart valve. For example, if the outcome of the comparison indicates hazardous calcification conditions, sensor data unit 90 determines that it is not feasible or safe to implant a balloon expandable prosthetic heart valve, which is expanded at high pressure. In such a case, other prosthetic heart valves can be utilized, which are not expanded by an inflatable medical balloon, and therefore may exert lower forces on the surrounding anatomy during deployment.

In one example, where at least one sensor 60 comprises a plurality of flex sensors, sensor data unit 90 determines a diameter indication of inflatable medical balloon 40, the diameter indication comprising a surface topography of inflatable medical balloon 40. The term “surface topography”, as used herein, means the way the surface is shaped. Particularly, as described above, flex sensors 60 indicate various depressions in the surface of inflatable medical balloon 40, thereby providing an indication of the surface topography.

In one example, where at least one sensor 60 comprises one or more diameter sensors and one or more force sensors, sensor data unit 90 compares changes in the diameter of inflatable medical balloon 40 to changes in the pressure between inflatable medical balloon 40 and the walls of patient lumen 95. If the pressure rises without a similar increase in diameter, it can be an indication that pressure against the tissue is increasing while inflatable medical balloon 40 has reached its maximum (or near maximum) expandible parameters, and should not be expanded further. Similarly, if the diameter increases without an increase in pressure, it can be an indication that inflatable medical balloon 40 has not yet reached its maximum expandible parameters and should thus may be safely expanded further. If the pressure increases along with a respective increase in diameter, it is also indicative that inflatable medical balloon 40 has not yet reached its maximum expandible parameters.

In one further example, sensor data unit 90 compares a predetermined function of the difference between the increase in pressure and the increase in diameter to a predetermined threshold. In one example, the predetermined function is a derivative of a curve plotted from: the increase values of the pressure and diameter; and/or the absolute values thereof.

It is known from material science that stress-strain curves describe the relationship between stress and strain, and are typically obtained by gradually applying a load (i.e., force) to a material and measuring the deformation caused thereto as a result of the applied load. Certain materials exhibit a behavior, in which the strain initially increases in a proportional ratio to the increase in the stress applied to the material (the linear elastic region). After a certain critical point (i.e., yield strength), the stress increase can cause the material to undergo plastic deformation and/or to suffer failure (e.g., fracture).

Is it contemplated that arterial and annular tissues (e.g., at a native heart valve) can exhibit certain similar behaviors, as described by stress-strain curves. For example, upon the initial application of a radial expanding force (i.e., stress) to the tissue, and more specifically to the annulus, the annular diameter can increase in a proportional ratio to the increase in the radial force applied thereto (an elastic region). After reaching a certain critical diameter, the tissue is expanded or stretched beyond its physiological limit, and therefore increasing the application of radial forces thereto can cause the tissue to sustain irreversible plastic deformation and/or suffer critical damage (e.g., rupture).

Sensor data unit 90, responsive to the outputs of sensor/s 60, is thus advantageously able to produce measurements which are indicative of the inflation diameter of inflatable medical balloon 40 and the radial forces exerted thereby on the surrounding tissue, within the desired implantation site, such as the site of a malfunctioning native valve within the heart. By simultaneously measuring the inflation diameter of inflatable medical balloon 40 and the forces exerted thereby on the surrounding tissue, it is possible to identify the critical expansion diameter for a prosthetic heart valve, in which a diameter larger than the critical diameter will exert increasing radial forces thereto, which can result in critical damage to the surrounding tissue. Advantageously, this identified critical expansion diameter is utilized to expand a prosthetic heart valve to a diameter optionally equal to or smaller than the critical diameter, in order to prevent possible tissue damage.

In one example, where sensor/s 60 comprise one or more thermal sensors, sensor data unit 90 receives data from the thermal sensor/s regarding the temperature at a plurality of predetermined locations juxtaposed with inflatable medical balloon 40. In one further example, where a plurality of thermal sensors 60 are secured to sensor position member 50, sensor data unit 90 receives data regarding the temperature at each of the plurality of predetermined locations, each of the plurality of predetermined locations juxtaposed with a respective thermal sensor 60. In another further example, where sensor/s 60 comprises one or more thermal cameras, the one or more thermal cameras 60 scan the surroundings of inflatable medical balloon 40 and output data regarding the temperature at each of the plurality of predetermined locations juxtaposed with inflatable medical balloon 40. In one example, the plurality of predetermined locations constitutes a predetermined area surrounding inflatable medical balloon 40. In one further example, the predetermined area exhibits a generally circular shape surrounding inflatable medical balloon 40.

Responsive to the received temperature data, sensor data unit 90 determines an indication of the rate of thermal dispersion between the inflatable medical balloon and the plurality of predetermined locations juxtaposed with inflatable medical balloon 40. Particularly, in one example sensor data unit 90 determines an indication of the rate of thermal dispersion by determining a heat gradient present between inflatable medical balloon 40 and the area surrounding inflatable medical balloon 40. In one example, the indication of the rate of thermal dispersion is determined responsive to a plurality of temperature measurements for each of the plurality of predetermined locations, taken at a plurality of predetermined time points.

In one example, the indication of the rate of thermal dispersion is determined responsive to the difference in temperature, optionally at the plurality of time points, between the inflation fluid of inflatable medical balloon 40 and the measured temperatures of the plurality of predetermined locations. In another example, the indication of the rate of thermal dispersion is determined responsive to the difference between the measured temperatures of the plurality of predetermined locations, optionally at the plurality of time points, and one or more predetermined temperature thresholds.

The term “rate of thermal dispersion”, as used herein, means the rate at which heat is transferred between each location and inflatable medical balloon 40. For example, a calcification exhibits a lower thermal conductivity than tissue, therefore there will be a lower rate of thermal dispersion between a calcification and inflatable medical balloon 40. In one example, the indication of the rate of thermal dispersion comprises the value of the rate of thermal dispersion. In another example, sensor data unit 90 compares the measured temperatures of the plurality of predetermined locations to each other and the indication of the rate of thermal dispersion is responsive to, and/or comprises, the difference between the measured temperatures. Particularly, if the measured temperature at a first location is lower than the measured temperature at a second location this is an indication that the rate of thermal dispersion is higher at the first location than at the second location.

In another example, sensor data 90 compares the measured temperatures of the plurality of predetermined locations to a predetermined temperature threshold and the indication of the rate of thermal dispersion for each location is responsive to, and/or comprises, the difference between the respective measured temperature and the predetermined temperature threshold. As described above, in one example a plurality of predetermined temperature thresholds are provided, and a plurality of temperature measurements are taken at a plurality of time points. In one further example, the temperature measurements from each time point are compared to a respective one of the plurality of predetermined temperature thresholds. In such an example, the indication of the rate of thermal dispersion for each location is responsive to, and/or comprises, a predetermined function of the respective comparisons through the different time points. It is noted that the above examples are not limiting, and the indication of the rate of thermal dispersion can be any suitable indication.

In one example, sensor data unit 90 determines, responsive to the determined rate of thermal dispersion for the plurality of predetermined locations, a map of the rate of thermal dispersion between inflatable medical balloon 40 and the plurality of predetermined locations. Particularly, in one example sensor data unit 90 generates a map of an area constituted of the plurality of predetermined locations, the information for each location of the map comprising the respective rate of thermal dispersion.

In one example, sensor data unit 90 identifies, responsive to the output of the at least one thermal sensor 60, a tissue gap at one or more of the plurality of predetermined locations. The term “tissue gap”, as used herein, means a space juxtaposed with inflatable medical balloon 40 that is void of tissue. A tissue gap can be present due to the wall of patient lumen 95 exhibiting an inward curvature. Tissue gaps can be a source of paravalvular leakage, therefore it is advantageous to identify these gaps.

In one further example, the tissue gap is identified responsive to the determined rate of thermal dispersion for the respective predetermined location. Particularly, in an example where the temperature of the inflation fluid is lower than the patient's body temperature, the tissue contacting inflatable medical balloon 40 will be cooled. Thus, an area where there is no tissue, i.e. where there is a tissue gap, will cool at a slower rate than an area where there is tissue in contact with inflatable medical balloon 40, i.e. it will exhibit a lower rate of thermal dispersion.

In one example, for each of the plurality of predetermined locations and responsive to the respective determined indication of the rate of thermal dispersion, sensor data unit 90 determines an extent of calcification at the respective predetermined location. Particularly, calcifications exhibit a lower thermal conductivity than surrounding tissue, and thus a lower rate of thermal dispersion. In one further example, sensor data unit 90 outputs an indication of the determined extent of calcification. In one example, the indication of the determined calcification extent comprises the size and/or position of each calcification. In another example, sensor data unit 90 determines a map of the area constituted of the plurality of predetermined locations, the map exhibiting indications of the extent of calcifications within the area. Thus, in such an example, the map comprises the indication of the determined extent of calcification at each of the plurality of predetermined locations. In one yet further example, the map further exhibits indications of any identified tissue gaps, as described above. In one example, a first predetermined temperature value, or values, is used for comparison to the measured temperatures to identify calcifications and a second predetermined temperature value, or values, is used for comparison to the measured temperatures to identify tissue gaps.

In another example, responsive the determined indication of the rate of thermal dispersion, sensor data unit 90 determines the density of the material juxtaposed with inflatable medical balloon 40. Particularly, the thermal dispersion of a material exhibits an inverse correlation with the density thereof, thus a lower thermal dispersion can indicate a higher density, and vice versa. An example of a high-density material is a calcification. In another example, responsive to the determined indication of the rate of thermal dispersion from two distinct locations along the native valve, sensor data unit 90 calculates flow velocities of blood (e.g., as in a Swan Ganz catheter).

As described above, in one example the temperature of the interior of inflatable medical balloon 40 is less than 31 degrees Celsius, optionally less than 21 degrees Celsius, optionally less than 10 degrees Celsius, optionally less than 4 degrees Celsius. The average body temperature is about 37 degrees Celsius, thus the lower the temperature of inflatable medical balloon 40 the higher the temperature difference will be between inflatable medical balloon 40 and the tissue of patient lumen 95. Advantageously, a higher temperature difference will allow a more accurate measurement of an indication of the thermal dispersion between inflatable medical balloon 40 and the tissue of patient lumen 95.

In one example, each heating element 93 generates heat at the predetermined temperature, for a predetermined heating period, and sensor data unit 90 determines the measured temperature at the one or more thermal sensors 60. Thus, in such an example the determined indication of the rate of thermal dispersion is between heating elements 93 and the plurality of predetermined locations. Particularly, the temperature measurements of thermal sensors 60 are affected by the rate at which the heat generated by heating elements 93 is diffused throughout the surrounding tissue and/or blood. In one further example, sensor data unit 90 controls each heating element 93 to generate heat.

In another further example, where a plurality of thermal sensors 60 are provided, sensor data unit 90 sequentially controls heating elements 93 to generate heat, i.e. a subset (one or more) of the plurality of heating elements 93 are operated each time. In such an example, responsive to the generated heat of each heating element 93, sensor data unit 90 determines the measured temperature at the respective thermal sensors adjacent thereto. Advantageously, this allows sensor data unit 90 to determine the rate of thermal dispersion in the vicinity of each heating element 93 without the measurements being affected by heat generated by other heating elements 93. Further advantageously, this allows communication of data with sensor data unit 90 to be multiplexed, thus using a reduced number of communication mediums 65.

In one example each thermal sensor 60 is associated with a respective heating element 93. For each of the plurality of predetermined locations, the determination of the indication of the rate of thermal dispersion is responsive to the temperature difference between a respective thermal sensor 60 and the associated heating element 93. Each thermal sensor 60 is not limited to being associated with a single heating element 93 and each heating element 93 is not limited to being associated with a single thermal sensor 60. Additionally, the association of thermal sensors 60 with respective heating elements 93 is not limited to an example where heating elements 93 are operated sequentially.

In another example, sensor data unit 90 determines an indication of the rate of thermal dispersion between each heating element 93 and one or more associated thermal sensors 60, when heating element 93 are generating heat at the same time. In one example, each thermal sensor 60 associated with a respective heating element 93 is adjacent thereto, however this is not meant to be limiting in any way. In another example, a first thermal sensor 60 associated with a respective heating element 93 can be positioned such that a second thermal sensor 60 is positioned between the first thermal sensor 60 and the respective heating element 93.

In another example, as described above, each thermal sensor 60, or each of a subset of thermal sensors 60, is arranged to alternately operate in a heating mode and a sensing mode. Particularly, in the heating mode, sensor data unit 90 controls the respective thermal sensor 60 to generate heat at a predetermined temperature, as described above in relation to heating element 93. In the sensing mode, sensor data unit 90 controls the respective thermal sensor 60 to sense the temperature at the respective thermal sensor 60. In such an example, at each of a plurality of predetermined time points, sensor data unit 90 controls a respective thermal sensor 60 to operate in the heating mode. Additionally, at the respective time point, sensor data unit 90 controls the thermal sensors 60 that are associated with the respective thermal sensor 60 operating in the heating mode to operate in the sensing mode. Thus, determining an indication of the rate of thermal dispersion is responsive to a temperature difference between the respective thermal sensor operating in the heating mode and the associated thermal sensors operating in the sensing mode.

It is noted that sensor data unit 90 can control more than one thermal sensor 60 to operate in the heating mode at each time point, while controlling the associated thermal sensors 60 of each of the thermal sensors 60 operating in the heating mode to operate in the sensing mode. Advantageously, by alternating thermal sensors 60 between the heating mode and the sensing mode, thermal dispersion can be measured by applying heat, yet without having to add additional heating elements, such as heating elements 93.

In another example, sensor data unit 90 outputs the determined indication of the rate of thermal dispersion. In one further example, sensor data unit 90 outputs the determined indication to a user display of handle 20, such as display 92. In another further example, sensor data unit 90 outputs the determined indication to an external display or system (not shown).

In another example, sensor data unit 90 outputs information associated with the determined indication of the rate of thermal dispersion. For example, as described above, the information associated with the determined indication of the rate of thermal dispersion can comprise, without limitation: the determined rate of thermal dispersion, an indication of a map of the thermal dispersion; an indication of the presence of a tissue gap at one or more of the plurality of predetermined locations; an indication that inflatable medical balloon 40 should be expanded more, or less, due to the presence or absence of a tissue gap; and/or an indication of the extent of calcification at one or more of the predetermined locations.

In one example, responsive to the determined rate of thermal dispersion, sensor data unit 90 determines whether there is a reduction in pulsatility. Particularly, when implanted into the native annulus, inflatable medical balloon 40 can reduce pulsatility when fully expanded, thereby reducing blood flow. A reduction in blood flow will reduce the rate of thermal dispersion. Therefore, sensor data unit 90 can identify a reduction in blood flow, and a reduction in pulsatility, responsive to the determined rate of thermal dispersion. In the event that sensor data unit 90 detects a reduction in pulsatility, in one example sensor data unit 90 outputs an indication that inflation of inflatable medical balloon 40 can be stopped. In an example, as described below, where sensor data unit 90 controls a pump to inflate inflatable medical balloon 40, sensor data unit 90 controls the pump to cease the inflation of inflatable medical balloon 40.

In one example, where a plurality of electrical conductance sensors 60 are provided, sensor data unit 90 determines, responsive to an output of electrical conductance sensors 60, an indication of the electrical conductance of the material juxtaposed with each electrical conductance sensor 60. In one further example, sensor data unit 90 further determines an extent of calcification of patient lumen 95 responsive the determined indications of electrical conductance. Particularly, the electrical conductance of tissue will be greater than the electrical conductance of calcifications. In another further example, sensor data unit 90 further identifies the presence of a gap in the tissue responsive to the determined indications of electrical conductance. Particularly, the electrical conductance of blood flowing through a gap between inflatable medical balloon 40 and patient lumen 95 will be different than the electrical conductance of tissue.

As described above, in one example each of a plurality of sets of sensors 60 is in communication with sensor data unit 90 via a respective communication medium 65. In such an example, communication between sensors 60 and sensor data unit 90 is multiplexed. Particularly, at each of a plurality of time points a respective sensor 60 of each set transmits data to sensor data unit 90. In one further example, the measurements are performed by the respective sensors 60 at the respective time points, i.e. at each time point only a respective sensor 60 of each set performs a measurements. In another example, the measurements are performed continuously, and the communication with sensor data unit 90 is performed one sensor at a time.

The mapping and/or imaging of the balloon/annulus described herein may be performed in real-time or near real-time during a balloon insertion and inflation procedure, which may be executed for a valvuloplasty or other repair procedure, or which may be executed prior to a prosthetic valve deployment (e.g., prior to a transcatheter aortic valve implantation (TAVI) procedure) in some examples. The above-described mapping may be performed in order to model the anatomy to provide measurements for use during a later deployment of the prosthetic valve (e.g., a distance to the annulus, an amount of inflation to use, a size of the prosthetic valve to use to fit the annulus, etc.) and/or to prepare the patient for the later deployment of the prosthetic valve (e.g., to identify obstacles in the annulus or the pathway to the annulus that may be targeted for clearing prior to deployment of the prosthetic valve). In other examples, the mapping and/or imaging of the balloon/annulus may be performed during deployment of a prosthetic valve in order to provide real-time feedback of the deployment status and allow optimization of the deployment size with the specific anatomy, as will be described further below.

After completing the inflation of inflatable medical balloon 40, the inflation fluid is removed therefrom thereby deflating inflatable medical balloon 40. Catheter 70 and inflatable medical balloon 40 are then retracted from patient lumen 95. Additionally, sensor position member 50 is similarly retracted. In an example where catheter 75 is provided, catheter 75 is retracted along with catheter 70. Advantageously, by securing sensor position member 50 to catheter 75, sensor position member 50 can be used with any standard balloon catheter, without having to make any manufacturing adjustments to the balloon or the catheter.

Although the above has been described in an example wherein sensor position member 50 is secured to catheter 75 and/or catheter 70, this is not meant to be limiting in any way. In another example (not shown), a delivery shaft is provided, sensor position member 50 being secured to the delivery shaft. In another example, as described above, sensor position member 50 is secured to inflatable medical balloon 40 at one or more predetermined locations. In another example, sensor position member 50 is tightly compressed over inflatable medical balloon 40 such that the friction between sensor position member 50 and inflatable medical balloon 40 prevents relative movement therebetween. Although sensor position member 50 is described herein as being secured to catheter 75 and/or catheter 70, catheter 70 and catheter 75 are preferably secured to handle 20, thus sensor position member 50 is preferably secured to handle 20. Advantageously, in the above examples, sensor position member 50 is secured to prevent relative movement between sensor position member 50 and inflatable medical balloon 40.

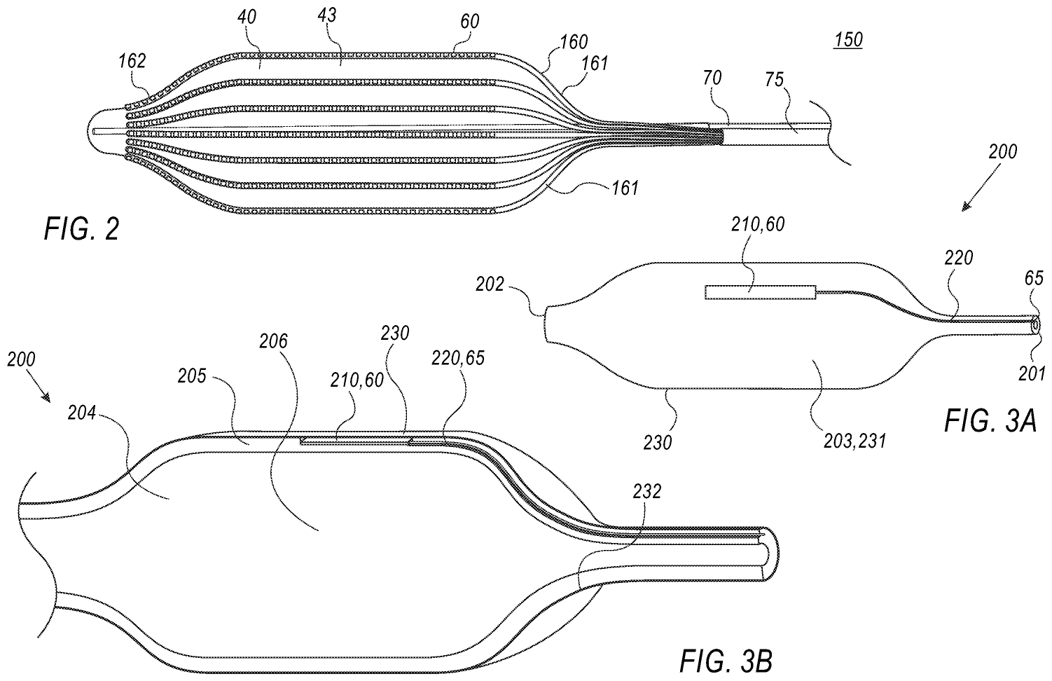

FIG. 2 illustrates a high-level perspective view of a medical balloon sensing assembly 150, in accordance with certain examples. Medical balloon sensing assembly 150 is in all respects similar to medical balloon sensing assembly 10, with the exception that sensor position member 50 is replaced with at least one sensor position member 160. For clarity, handle 20 is not illustrated. In one example, as illustrated, each position member 160 exhibits a proximal end 161 and a distal end 162. In one example, proximal end 161 of each position member 160 is secured to catheter 75 and distal end 162 of each position member 160 is juxtaposed with outer face 43 of inflatable medical balloon 40. In another example (not shown), proximal end 161 of each position member 160 is secured to catheter 70 and distal end 162 of each position member 160 is juxtaposed with outer face 43 of inflatable medical balloon 40.

In one example, each position member 160 is elongated, extending from the respective proximal end 161 to the respective distal end 162. In another example, distal end 162 of each position member 160 is attached to outer face 43 of inflatable medical balloon 40. One or more sensors 60 are secured to each position member 160. In one example, a plurality of sensors 60 are positioned along each sensor position member 160. The operation of medical balloon sensing assembly 150 is in all respects similar to the operation of medical balloon sensing assembly 10, and in the interest of brevity will not be described further.

Although the above has been described wherein sensor position members 160 are secured to catheter 75 and/or catheter 70, this is not meant to be limiting in any way. In another example (not shown), a delivery shaft is provided, sensor position members 160 secured to the delivery shaft. Although sensor position members 160 are described herein as being secured to catheter 75 and/or catheter 70, catheter 75 and catheter 75 are secured to handle 20, thus sensor position members 160 are secured to handle 20. Although the above has been described wherein sensor position members 160 extend longitudinal along inflatable medical balloon 40, this is not meant to be limiting in any way. In another example (not shown), one or more sensor position members 160 extend circumferentially around inflatable medical balloon 40, such as in a circular shape.

Although the above has been described in relation to examples where sensors 60 are positioned on a sensor position member 50 and/or sensor position members 160, this is not meant to be limiting in any way. In another example, sensors 60 are positioned on outer surface 43 and/or inner surface 44 of inflatable medical balloon 40.

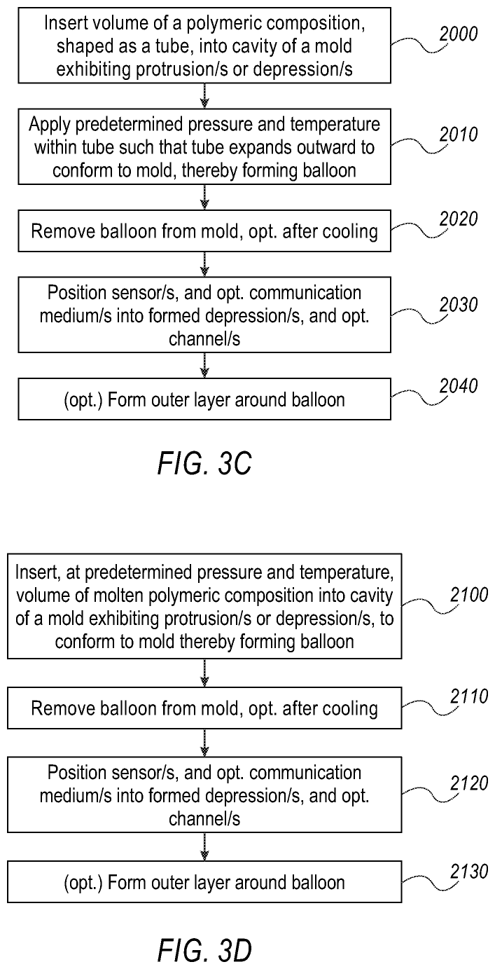

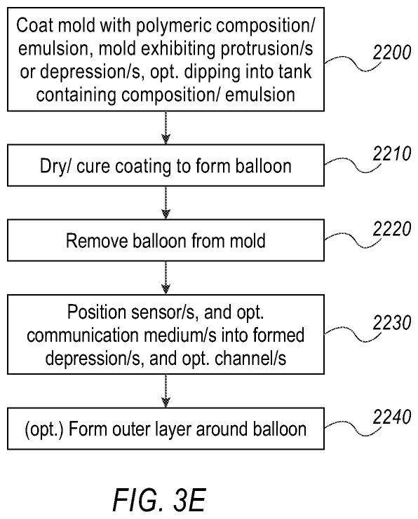

FIG. 3A illustrates a high-level perspective view of an inflatable medical balloon 200 and FIG. 3B illustrates a high-level perspective cut-away view of inflatable medical balloon 200, in accordance with certain examples, FIGS. 3A-3B described together. In one example, inflatable medical balloon 200 replaces: inflatable medical balloon 40 and sensor position member 50 of medical balloon sensing assembly 10; and/or inflatable medical balloon 40 and sensor position members 160 of medical balloon sensing assembly 150. Inflatable medical balloon 200 exhibits: a proximal end 201; a distal end 202 opposing proximal end 201; an outer face 203; an inner face 204 opposing outer face 203; and a material layer 205, material layer 205 defined between inner face 204 and outer face 203. Outer face 203 and material layer 205 exhibit one or more depressions 210. Each depression 210 defines a predetermined volume which is void of the material of material layer 205. In one example, inflatable medical balloon 200 further exhibits one or more channels 220, each channel 220 extending from a respective depression 210 towards proximal end 201. In one further example, each channel 220 is shaped as a groove.

In another example, inflatable medical balloon 200 further exhibits an outer layer 230, outer layer 230 enclosing outer face 203. Particularly, outer layer 230 exhibits an outer face 231 and an inner face 232 opposing outer face 231. In such an example, inner face 232 of outer layer 230 faces outer face 203 of inflatable medical balloon 200 and outer face 231 faces away from inflatable medical balloon 200. Although outer layer 230 is illustrated as surrounding the entire circumference of inflatable medical balloon 200 and extending from proximal end 201 to distal end 202, this is not meant to be limiting in any way. In another example (not shown), outer layer 230 covers only a portion of outer face 203 of inflatable medical balloon 200.

In one example, outer layer 230 is formed by dip-coating, chemical vapor deposition, extrusion and/or any other suitable methods known to those skilled in the art. In another example, outer layer 230 comprises a polymer. In one further example, outer layer 230 is formed as a parylene coating. In one example, outer layer 230 is formed by dip coating a mold with a polymer composition or emulsion and then attaching outer layer 230 to inflatable medical balloon 200. In another example, outer layer 230 is formed by dip coating inflatable medical balloon 200 with a polymer composition or emulsion. In another example, outer layer 230 is formed by blow molding or injection molding, and then attaching outer layer 230 to inflatable medical balloon 200.

Although inflatable medical balloon 200 is illustrated as exhibiting only a single depression 210, this is not meant to be limiting in any way, and any number of depressions 210 can be provided without exceeding the scope of the disclosure. As described above in relation to sensors 60 of medical balloon sensing assembly 10, in one example a plurality of depressions 210 are provided, depressions 210 separated from each other by predetermined spacings. Additionally, although depression 210 is illustrated as being rectangular shaped, this is not meant to be limiting in any way, and depression 210 can be shaped in any suitable way, without exceeding the scope of the disclosure. Inner face 204 defines an enclosure 206.

Although inflatable medical balloon 200 is illustrated as being generally cylindrical shaped, this is not meant to be limiting in any. In another example (not shown), inflatable medical balloon 200 can be shaped as a cone, an ellipse, a sphere, or any other suitable shape known to those skilled in the art.

As described above in relation to medical balloon sensing assembly 10, one or more sensors 60 are provided. Each sensor 60 is positioned within a respective depression 210 such that the respective sensor 60 is embedded within the material of inflatable medical balloon 200. In one example, depressions 210 are shaped and dimensioned such that each sensor 60 is completely disposed within the respective depression 210, i.e. completely embedded within the material of inflatable medical balloon 200. In another example (not shown), one or more sensors 60 partially protrude outwards from the respective depressions 210. In one example, each sensor 60 is positioned within a respective depression 210. In another example (not shown), some of sensors 60 are positioned within depressions 210 and the rest of sensors 60 are positioned on outer face 203 of inflatable medical balloon 200 and/or on outer face 231 of outer layer 230.

Although the illustrated depression 210 is shown as having positioned therein one sensor 60, this is not meant to be limiting in any way, and any number of sensors 60 can be positioned within each depression 210 without exceeding the scope of the disclosure. In one example, a respective communication medium 65 is positioned within each channel 220 and is coupled to a respective sensor 60. Each communication medium 65 provides communication between the respective sensor 60 and a sensor data unit (not shown). In one further example, each communication medium 65 is arranged to allow: electrical communication via a conductive material, such as a wire; and/or optical communication, e.g. via an optical fiber. Alternatively, as described above, each sensor 60 is in wireless communication with the sensor data unit.

In one example (not shown), where a sensor 60 comprises a radially translatable member connected to a linear displacement sensor, as described above, depression 220 extends along the circumference of inflatable medical balloon 200 such that the radially translatable member is positioned within depression 210. In one example (not shown), where each depression 210 has positioned therein a plurality of sensors 60, a plurality of communication mediums 65 are positioned within the respective channel 220, each communication medium 65 coupled to a respective sensor 60.

In the example where outer layer 230 is provided, sensors 60 are enclosed within the respective depressions 210 by outer layer 230 such that each sensor 60 faces inner face 232 of outer layer 230. Similarly, in such an example, communication mediums 65 are enclosed within the respective channels 220 by outer layer 230 such that each communication medium 65 faces inner face 232 of outer layer 230.

Although inflatable medical balloon 200 is described and illustrated herein in an example where depressions 210 and channels 220 are in the outer portion of material layer 205, this is not meant to be limiting in any way. In another example (not shown), depressions 210 and/or channels 220 are in an inner portion of material layer 205, i.e. they face enclosure 206. Additionally, although inflatable medical balloon 200 is illustrated herein in an example where depressions 210 and channels 220 do not alter the shape of inner face 204, this is not meant to be limiting in any way. In another example (not shown), depressions 210 and/or channels 220 extend into enclosure 206.

The operation of inflatable medical balloon 200 is in all respects similar to the operation of inflatable medical balloon 40. Particularly, as described above in relation to inflatable medical balloon 40, inflatable medical balloon 200 inflates when inflation fluid is injected therein. As inflatable medical balloon 200 is inflated, sensors 60 measure diameter, force and/or pressure indicators, as described above. The operation of inflatable medical balloon 200 will not be described further, for the sake of brevity. Although inflatable medical balloon 40 and inflatable medical balloon 200 are illustrated herein as distinct examples, this is not meant to be limiting in any way. In one example (not shown), inflatable medical balloon 40 exhibits one or more depressions 210 with some of sensors 60 positioned within depressions 210 and the rest of sensors 60 positioned on sensor position member 50 and/or sensor position members 160. In another example (not shown), inflatable medical balloon 200 is associated with a sensor position member 50 and/or one or more sensor position members 160, such that some of sensors 60 are positioned within depressions 210 and the rest of sensors 60 are positioned on sensor position member 50 and/or sensor position members 160.

FIGS. 3C-3E each illustrate a high-level flow chart of a respective method of constructing inflatable medical balloon 200, in accordance with certain examples. Inflatable medical balloons are typically manufactured independently of the associated catheter and are then secured to the catheter with an adhesive or via other bonding methods. One standard balloon manufacture technique comprises utilizing an extruded polymeric tube which may be radially expanded into a mold cavity by blow molding or by free-blowing. In a standard blow molding process, a polymeric tube is blown radially outward under the action of internal pressure and heat within a balloon mold, until the expanding polymer tube forms a balloon shape that is constrained by the mold cavity wall(s). The polymer tube may either be simultaneously stretched in the radial and axial directions, or first stretched axially and then radially. In another manufacturing technique, a molten polymeric composition is injected into the cavity of a mold at a predetermined pressure and temperature. In another manufacturing technique, a mandrel is dipped into the liquid polymer, thereby forming the balloon around the mandrel. The final balloon is typically characterized by having a balloon wall exhibiting a uniform thickness in an expanded (inflated) and/or unexpanded (relaxed/deflated) state.

Although a range of materials (and combinations of materials) are capable of being inflated at the pressures needed to perform functions, polymeric materials for layers or compounds are particularly well suited for applications. They have the flexibility to shrink to small diameters and the elasticity to expand without bursting. Polymeric materials include, for example, thermoplastic and thermoset polymers. Such polymers include, for example, polyethylene terephthalate (PET), nylon, Pebax®, polyurethane, polyetherurathane, polyvinylpyrrolidone (PVP), polyethylene oxide (PEO), high-density polyethylene (HDPE) and low-density polyethylene (LDPE).

FIG. 3C illustrates a high-level flow chart of a first method of constructing inflatable medical balloon 200, in accordance with certain examples. In stage 2000, a predetermined volume of a polymeric composition shaped as a tube is inserted into a cavity of a mold, the mold exhibiting one or more protrusions extending into the cavity. Alternatively, the mold exhibits one or more depressions that form an expansion of the cavity. In one example, the mold exhibits a plurality of protrusions or a plurality of depressions. In one further example, the protrusions, or the depressions, exhibit a variety of shapes and dimensions. For example, as described above, some of the protrusions, or depressions, are shaped and dimensioned to form depressions 210 and some of the protrusions, or depressions, are shaped and dimensioned to form channels 220.

In one example, the tube is formed of one or more polymers. In another example, the tube is formed by an extrusion process. In one example, the tube is stretched axially and/or radially prior to placement within the mold. In one further example, the axial and radial stretching is performed simultaneously. In one example, the tube is pre-cut into predetermined dimensions prior to placement within the mold. In another example, the cavity of the mold exhibits a center section exhibiting a first radial diameter and a pair of side sections each exhibiting a narrower radial diameter.

In stage 2010, a predetermined pressure, at a predetermined temperature, is applied within an opening of the tube thereby causing the tube to radially expand outward and conform to the shape and dimensions of the mold, as known to those skilled in the art. The conformance to the shape and dimensions of the mold defines inflatable medical balloon 200. The one or more protrusions, or depressions, of the mold form depression/s 210 and optionally channels 220. In one example, the predetermined pressure is applied by blowing a hot gas (e.g. air) at the predetermined temperature within the opening of the tube.

In stage 2020, inflatable medical balloon 200 is removed from the mold. In one example, prior to being removed from the mold, inflatable medical balloon 200 is cooled to a predetermined cooling temperature. In one example, where the mold exhibits one or more depressions, inflatable medical balloon 200 is turned inside out such that depressions 210, and optionally channels 220, face outwards.

In stage 2030, one or more sensors 60 are positioned within the respective depressions 210, as described above. In one example, as further described above, one or more communication mediums 65 are positioned within the respective channels 220. Placement of sensors 60 and/or communication mediums 65 can be performed before or after inflatable medical balloon 40 is removed from the mold. For example, in an example where the mold comprises one or more depressions, sensors 60 and/or communication mediums 65 can be positioned while still on the mold such that the mold provides support during placement. In another example, inflatable medical balloon 200 is further secured to a catheter by melting, utilizing appropriate adhesives or any other suitable method, as known to those skilled in the art.

In optional stage 2040, an outer layer is formed around at least a portion of an outer face of inflatable medical balloon 200, as described above in relation to outer layer 230. In one example, the outer layer is further formed around at least a portion of a catheter that secures inflatable medical balloon 200.

Although the method of FIG. 3C is described for the sake of clarity in relation to inflatable medical balloon 200, this is not meant to be limiting in any way, and the method of stages 2000-2040 can be utilized to form any type of inflatable medical balloon without exceeding the scope of the disclosure.

FIG. 3D illustrates a high-level flow chart of a second method of constructing inflatable medical balloon 200, in accordance with certain examples. In stage 2100, a predetermined volume of molten polymeric composition is inserted into a cavity of a mold at a predetermined pressure and temperature, as known to those skilled in the art. As described above in relation to stage 2000, the mold exhibits one or more protrusions extending into the cavity, or one or more depressions that form an expansion of the cavity. In one example, the molten polymer composition is injected into the cavity of the mold. The inserted polymer composition conforms to the shape and dimensions of the mold thereby forming inflatable medical balloon 200, as known to those skilled in the art. As described above, the one or more protrusions, or depressions, of the mold form depression/s 210 and optionally channels 220.

In another example, the molten material is manufactured utilizing an injection molding machine, as known to those skilled in the art. In one further example, the injection molding machine comprises an injection ram or a screw-type plunger similar to the one used in extrusion processes. In another further example, the injection molding machine comprises 3-dimensional (3D) printing technologies, as known to those skilled in the art. In one example, the manufacturing of the molten material and the injection into the mold can be performed within a single injection molding machine. In another example, manufacturing of the molten material is performed with a first machine and the injection into the mold is performed with a separate machine.

In stage 2110, inflatable medical balloon 200 is removed from the mold. In one example, prior to being removed from the mold, inflatable medical balloon 200 is cooled to a predetermined cooling temperature until it hardens. In one example, where the mold exhibits one or more depressions, inflatable medical balloon 200 is turned inside out such that depressions 210, and optionally channels 220, face outwards.

In stage 2120, one or more sensors 60 are positioned within the respective depressions 210, as described above. In one example, as further described above, one or more communication mediums 65 are positioned within the respective channels 220. Placement of sensors 60 and/or communication mediums 65 can be performed before or after inflatable medical balloon 40 is removed from the mold. For example, in an example where the mold comprises one or more depressions, sensors 60 and/or communication mediums 65 can be positioned while still on the mold such that the mold provides support during placement. In another example, inflatable medical balloon 200 is further secured to a catheter by melting, utilizing appropriate adhesives or any other suitable method, as known to those skilled in the art.

In optional stage 2130, an outer layer is formed around at least a portion of an outer face of inflatable medical balloon 200, as described above in relation to outer layer 230. In one example, the outer layer is further formed around at least a portion of a catheter that secures inflatable medical balloon 200.

Although the method of FIG. 3D is described for the sake of clarity in relation to inflatable medical balloon 200, this is not meant to be limiting in any way, and the method of stages 2100-2130 can be utilized to form any type of inflatable medical balloon without exceeding the scope of the disclosure.

FIG. 3E illustrates a high-level flow chart of a third method of constructing inflatable medical balloon 200, in accordance with certain examples. In stage 2200, a mold is coated with a polymeric composition or emulsion to form inflatable medical balloon 200, as known to those skilled in the art. In one example, the mold comprises a mandrel. In another example, the polymeric composition/emulsion is in a liquid uncured state. In one example, the polymeric composition/emulsion comprises silicone, polyurethane, polyisoprene, polyvinyl, polyether, polydimethylsiloxane, elastomers, latex rubber and/or nitrile rubber, and variations and combinations thereof. The mold exhibits one or more protrusions, or one or more depressions, as described above in relation to stages 2000 and 2100. As described above, the protrusions/depressions are shaped and dimensioned to form one or more depressions 210 and optionally one or more channels 220.

In one example, the coating is performed by dipping the mold into a tank containing the polymeric composition/emulsion. The terms “dipping” and “immersing” are interchangeable. In another example, the mold and/or the polymeric composition/emulsion are heated to at least one predetermined temperature. Responsive to the coating, a layer of liquid polymeric material is formed over the outer surface of the mold, as known to those skilled in the art. In one example, the formed layer is a thin film layer.

In stage 2210, the polymeric layer encompassing the mandrel is dried and/or cured, thereby forming a cured/solid polymeric inflatable medical balloon 200 attached thereto. The term “cured”, as used herein, refers to a process where the liquid polymeric layer undergoes toughening or hardening of the polymer material by cross-linking of polymer chains in order to form a solid polymeric layer. As described above, one or more depressions 210, and optionally one or more channels 220, are formed in a surface of inflatable medical balloon 200 by the protrusions/depressions of the mold. In one example, excess ingredients and/or solvents are removed from the polymeric layer. These can be removed prior, during and/or after the curing and/or drying process. In one example, the polymeric layer is further cooled.

In stage 2220, inflatable medical balloon 200 is removed from the mold. In one example, inflatable medical balloon 200 is peeled from the mold. In one example, where the mold exhibits one or more protrusions, inflatable medical balloon 200 is turned inside out such that depressions 210, and optionally channels 220, face outwards.

In stage 2230, one or more sensors 60 are positioned within the respective depressions 210, as described above. In one example, as further described above, one or more communication mediums 65 are positioned within the respective channels 220. In another example, inflatable medical balloon 200 is further secured to a catheter by melting, utilizing appropriate adhesives or any other suitable method, as known to those skilled in the art.

In optional stage 2240, an outer layer is formed around at least a portion of an outer face of inflatable medical balloon 200, as described above in relation to outer layer 230. In one example, the outer layer is further formed around at least a portion of a catheter that secures inflatable medical balloon 200.

Although the method of FIG. 3E is described for the sake of clarity in relation to inflatable medical balloon 200, this is not meant to be limiting in any way, and the method of stages 2200-2240 can be utilized to form any type of inflatable medical balloon without exceeding the scope of the disclosure.

Advantageously, unlike the standard balloon manufacturing techniques known in the art, resulting in the formation of balloons having uniform thickness, the above methods provide an inflatable medical balloon exhibiting respective depressions for placement of sensors such that sensors are provided without increasing the dimensions of the balloon. Additionally, an outer layer can cover the sensors such that the balloon exhibits a smooth outer face, without any protrusions caused by sensors positioned on the outer face of the balloon. Such configurations can be advantageous for balloons supporting external expandable structures such as stents or prosthetic valves, so as to reduce the risk of frictional contact between the external structures and the sensors attached to the balloon, which can result in accidental detachment of the sensors from the balloon and/or damage to the sensors.

Although the above has been described in relation to an example where sensors 60 are embedded into respective depressions 210 of inflatable medical balloon 200, this is not meant to be limiting in any way, and sensors 60 can be embedded in an inflatable medical balloon in any suitable way. In another example, the inflatable medical balloon is formed of a woven fabric and sensors 60 are positioned within the woven structure of the fabric.

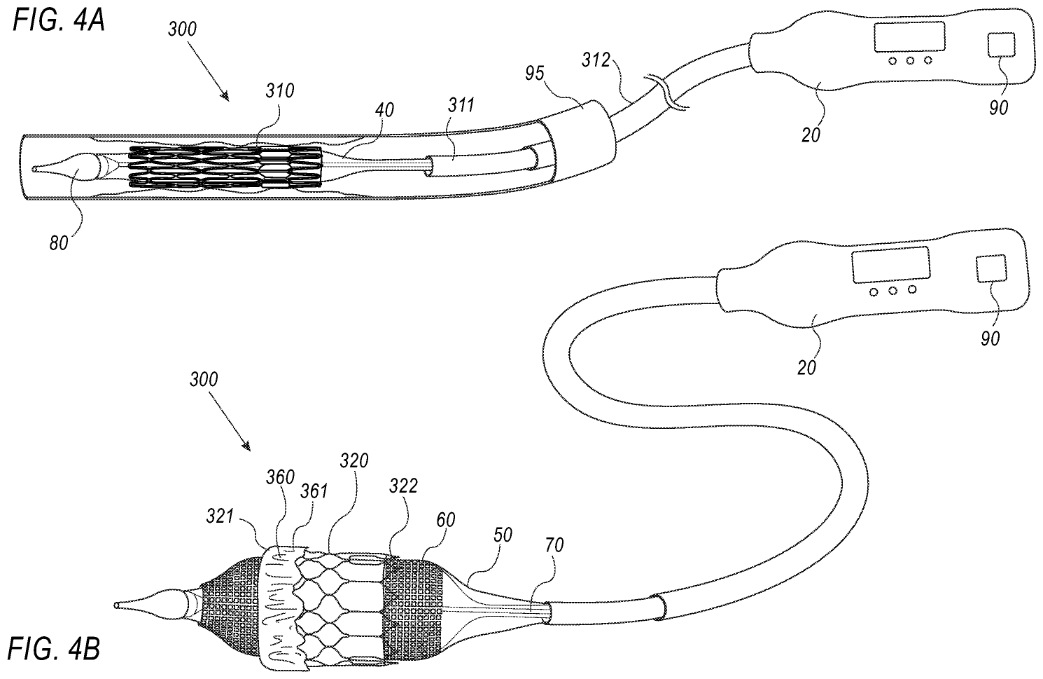

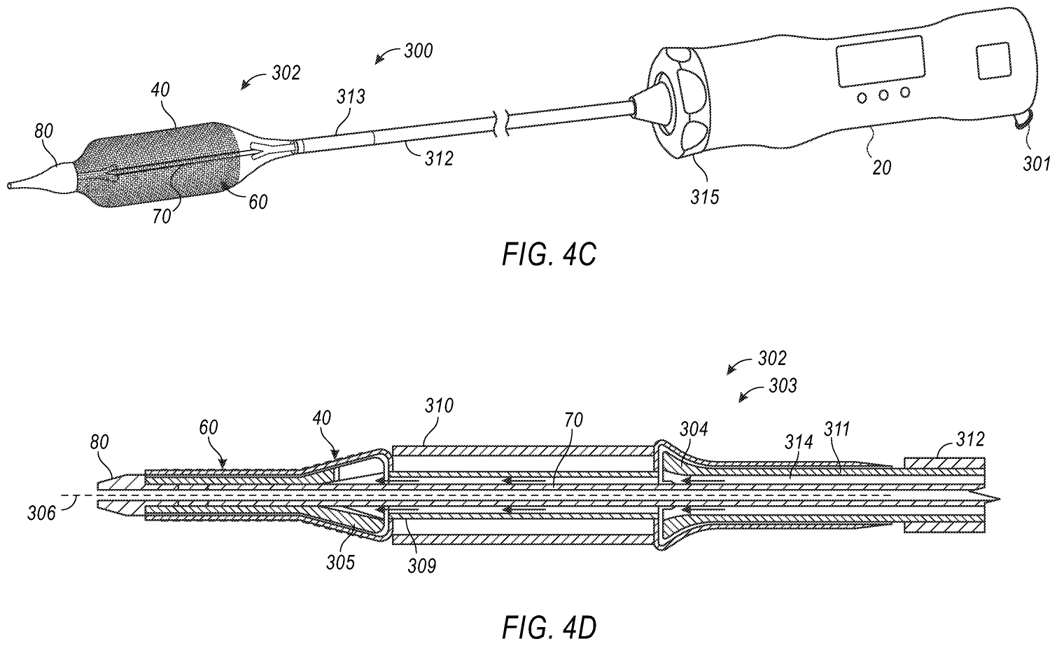

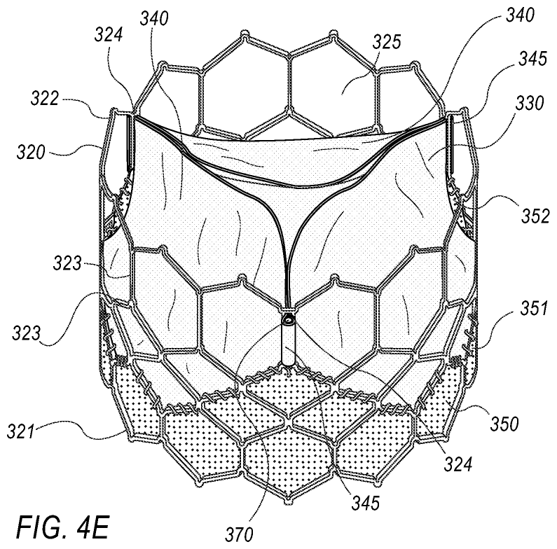

FIGS. 4A-4E illustrate high-level perspective views of various parts and states of a medical balloon sensing assembly 300, in accordance with certain examples. Medical balloon sensing assembly 300 is in all respects similar to medical balloon sensing assembly 10, with the exception that medical balloon sensing assembly 300 further comprises a prosthetic valve 310, a delivery shaft 311 and an optional outer shaft 312. The term “prosthetic valve”, as used herein, refers to any type of a prosthetic valve deliverable to a patient's target site, which is radially expandable and compressible between a radially compressed, or crimped, state, and a radially expanded state. Although medical balloon sensing assembly 300 is described as comprising a prosthetic valve 310, this is not meant to be limiting in any way. In another example (not shown), a stent, graft or other balloon expandable device is provided instead of prosthetic valve 310. Alternatively, a prosthetic valve constructed differently than prosthetic valve 310 can be provided, without exceeding the scope of the disclosure. In one example, prosthetic valve 310 is constructed so as to be mounted within the native aortic valve, the native mitral valve, the native pulmonary valve and/or the native tricuspid. Sensor position member 50 and sensors 60 are not shown in FIG. 4A for simplicity.

In one example, as illustrated in FIG. 4C, handle 20 further comprises a fluid port 301. In one further example, fluid port 301 is in fluid communication with a reservoir comprising an inflation fluid, as described below in relation to medical balloon sensing assembly 500. In another further example, fluid port 301 is shaped and dimensioned such that a syringe and/or manual pump can be connected thereto. In another example, as further illustrated in FIG. 4C, handle 20 further comprises an adjustment member 315, such as a rotatable knob. In one further example, adjustment member 315 is operatively coupled to a proximal end portion of a pull wire (not shown). The pull wire extends distally from handle 20 through delivery shaft 311 and has a distal end portion affixed to delivery shaft 311 at or near a distal end 313 of delivery shaft 311.

In one example, as illustrated in FIG. 4D, catheter 70 extends distally beyond optional outer shaft 312 and delivery shaft 311, and through inflatable medical balloon 40. In another example, inflatable medical balloon 40 is supported on a balloon shoulder assembly 303. Balloon shoulder assembly 303 exhibits a proximal shoulder 304 connected to a distal end of delivery shaft 311 and a distal shoulder 305 mounted on catheter 70. Proximal end 41 of inflatable medical balloon 40 surrounds and/or is folded over proximal shoulder 304 and distal end 42 of inflatable medical balloon 40 surrounds and/or is folded over distal shoulder 305. In one example, proximal end 41 of inflatable medical balloon 40 is secured to the outer surface of delivery shaft 311. In another example, distal end 42 of inflatable medical balloon 40 is secured to the outer surface of nosecone 80 (as shown), which is optionally mounted on, or coupled to, catheter 70.

In one example, nosecone 80 and distal shoulder 305 are a one-piece or unitary component, that is, nosecone 80 is a distal portion of the unitary component and distal shoulder 305 is a proximal portion of the unitary component. In another example, nosecone 80 and distal shoulder 305 are separate components, and each can be mounted on catheter 70 next to each other or at axially spaced locations.

In one example, proximal shoulder 304 and distal shoulder 305 are spaced apart from one another, in an axial direction relative to a central longitudinal axis 306 of medical balloon sensing assembly 300. As a result, inflatable medical balloon 40 defines a valve-retaining portion 309 in the space that separates proximal shoulder 304 and distal shoulder 305 (e.g., between flared ends of proximal shoulder 304 and distal shoulder 305), central longitudinal axis 306 extending through valve-retaining portion 309. As shown in FIG. 4D, prosthetic valve 310 is in one example crimped onto valve retaining portion 309 of inflatable medical balloon 40, between proximal shoulder 304 and distal shoulder 305, thereby preventing or reducing axial movement of prosthetic valve 310 relative to inflatable medical balloon 40 during insertion of medical balloon sensing assembly 300 into the patient and delivery of prosthetic valve 310 to the target implantation site.

In one example, the outer diameter of catheter 70 is sized such that an annular space 314 is defined between catheter 70 and delivery shaft 311 along the length of delivery shaft 311. In one further example, annular space 314 is in fluid communication with one or more fluid passageways of medical sensing assembly 300 which can be in fluid communication with a fluid source (e.g., a syringe) that can inject an inflation fluid (e.g., saline) into the delivery device, optionally via fluid port 301. In this way, fluid from the fluid source can flow through the one or more fluid passageways, through annular space 314, and into inflatable medical balloon 40 to inflate the balloon 40 and expand and deploy the prosthetic valve 310.

Catheter 70, delivery shaft 311 and optional outer shaft 312 of medical balloon sensing assembly 300 can be formed from any of various suitable materials, such as nylon, braided stainless steel wires, or a polyether block amide (commercially available as Pebax®). In one example, delivery shaft 311 and optional outer shaft 312 of medical balloon sensing assembly 300 have longitudinal sections formed from different materials in order to vary the flexibility of the shafts along their lengths. In another example, catheter 70 has an inner liner or layer formed of Teflon® to minimize sliding friction with a guide wire (not shown). In another example, delivery shaft 311 and optional outer shaft 312 of medical balloon sensing assembly 300 are axially and/or rotatably movable relative to each other and/or handle 20.

In one example, as further illustrated in FIGS. 4B and 4E, prosthetic valve 310 comprises: a frame 320 exhibiting an inflow end portion 321 and an outflow end portion 322; a leaflet assembly 330 comprising one or more leaflets 340; an inner skirt 350 exhibiting an outer face 351 and an inner face 352, inner face 352 opposing outer face 351; and an outer skirt 360 exhibiting an outer face 361 and an inner face (not shown), the inner face opposing outer face 361. In one example, frame 320 comprises a plurality of interconnected struts 323. In one further example, struts 323 are arranged in a lattice-type pattern.

In one example, frame 320 is made of any of various suitable plastically-expandable materials (e.g., stainless steel, a cobalt chromium alloy, etc.) or self-expanding materials (e.g., a nickel titanium alloy (NiTi), such as nitinol) as known in the art. When constructed of a plastically-expandable material, frame 320 (and thus prosthetic valve 310) is in one example crimped to a radially collapsed configuration on a delivery catheter and then expanded inside a patient by an inflatable medical balloon or equivalent expansion mechanism. When constructed of a self-expandable material, frame 320 (and thus prosthetic valve 310) is in one example crimped to a radially collapsed configuration and restrained in the collapsed configuration by insertion into a sheath or equivalent mechanism of a delivery catheter. Once inside the body, prosthetic valve 310 can be advanced from the delivery sheath, which allows prosthetic valve 310 to expand to its functional size. In some cases, a prosthetic valve equipped with a self-expandable frame can be used in combination with an inflatable medical balloon, wherein the valve can self-expand to a certain degree (i.e., a certain expansion diameter), while a balloon may be inflated to promote additional expansion of the prosthetic valve against the native anatomy.

In another example, struts 323 are constructed of any suitable plastically-expandable materials. Suitable plastically-expandable materials that can be used to form struts 323 include, without limitation, stainless steel, a biocompatible, high-strength alloys (e.g., a cobalt-chromium or a nickel-cobalt-chromium alloys), polymers, or combinations thereof. In one example, struts 323 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding prosthetic valve 310 and its various components are described in PCT Patent Application Publication No. WO 2018/222799, published Dec. 6, 2018 and entitled “Sealing member for prosthetic heart valve”, the entire contents of which are incorporated herein by reference.

In one example, frame 320 is formed from a single piece of material, such as a metal tube, via various processes such as, but not limited to, laser cutting, electroforming, and/or physical vapor deposition, while retaining the ability to collapse/expand radially.

In one example, one or more of struts 323 comprises an opening 324. In one example, each opening 324 is at, or near, outflow end portion 322. In one example, the number of openings 324 is equal to the number of leaflets 340. In another example, prosthetic valve 310 further comprises one or more commissure posts (not shown), each commissure post being attached to, or integrally formed with, the frame. In another example, each leaflet 340 includes two opposing upper tabs, the tabs of each pair of adjacent leaflets 340 being positioned within a respective opening 324. In one further example, a wedge 370 is positioned between the portions of the tabs disposed externally to frame 320. Wedges 370 prevent these portions of the tabs from sliding inwards through the respective openings 324. In one example, inner skirt 350 and/or outer skirt 360 are constructed of various biocompatible materials, such as, but not limited to, various synthetic materials (e.g., polyethylene terephthalate) or natural tissue (e.g. pericardial tissue).

Leaflet assembly 330 is illustrated as comprising 3 leaflets 340, however this is not meant to be limiting in any way. Particularly, while three leaflets 340 arranged to collapse in a tricuspid arrangement similar to the native aortic valve are illustrated, it will be clear that prosthetic valve 310 can include any other number of leaflets 340, such as two leaflets configured to collapse in a bicuspid arrangement similar to the native mitral valve, or more than three leaflets, depending upon the particular application. In one example, leaflets 340 are made of a flexible material, derived from biological materials (e.g., bovine pericardium or pericardium from other sources), bio-compatible synthetic materials, or other suitable materials as known in the art and described, for example, in: U.S. Pat. No. 6,730,118, issued May 4, 2004, and entitled “Implantable prosthetic valve”; U.S. Pat. No. 6,767,362, issued Jul. 27, 2004, and entitled “Minimally-invasive heart valves and methods of use”; and U.S. Pat. No. 6,908,481, issued Jun. 21, 2005, and entitled “Value prosthesis for implantation in body channels”, the entire contents of each of which are incorporated herein by reference.

In one example, leaflet assembly 330 is secured to frame 320 at, or near, outflow end portion 322. In one example, leaflets 340 are formed of a continuous piece of material. In another example, separate leaflets 340 are provided, and are separately secured to frame 320 and/or secured to each other.

Each pair of leaflets 340 is secured (for example, at their tabs) to a respective opening 324 which serves as a commissure window, thereby defining a commissure 345, i.e. an area where two leaflets 340 meet. Although leaflets 340 are illustrated and described herein as extending through and being secured to openings 324, this is not meant to be limiting in any way. In another example, leaflets 340 can be directly attached to frame 320 or attached to other structural elements connected to frame 320, such as commissure posts, without exceeding the scope of the disclosure. Further details regarding prosthetic valves, including the manner in which leaflets may be mounted to their frames, are described in: U.S. Pat. No. 7,393,360, issued Jul. 1, 2008, and entitled “Implantable prosthetic valve”; U.S. Pat. No. 7,510,575, issued Mar. 31, 2009, and entitled “Implantable prosthetic valve”; and U.S. Pat. No. 7,993,394, issued Aug. 9, 2011, and entitled “Low profile transcatheter heart valve”, the entire contents of each of which are incorporated herein by reference.

Although prosthetic valve 310 is illustrated and described herein as comprising inner skirt 350 and outer skirt 360, this is not meant to be limiting in any way. In another example (not shown), one or both of inner skirt 350 and outer skirt 360 are not provided. In one example, inner skirt 350 is arranged to function as a sealing member to prevent or decrease perivalvular leakage. In another example, leaflets 340 are secured to inner skirt 350 such that inner skirt 350 functions as an anchoring region for anchoring leaflets 340 to frame 320 and/or functions to protect leaflets 340 against damage which may be caused by contact with frame 320, for example during valve crimping or during working cycles of prosthetic valve 310. In one example, the edge of leaflets 340 secured to inner skirt 350 exhibits an undulating, curved and/or scalloped shape. In one example, outer skirt 360 is arranged to function as a sealing member retained between frame 320 and the surrounding tissue of the native annulus against which prosthetic valve 310 is mounted, thereby reducing risk of paravalvular leakage past prosthetic valve 310.

The term “outflow”, as used herein, refers to a region of prosthetic valve 310 through which the blood flows through and out of prosthetic valve 310. The term “inflow”, as used herein, refers to a region of prosthetic valve 310 through which the blood flows into prosthetic valve 310.

Although commissures 345 are illustrated and described herein as being secured to the opening 324 of frame 320 by wedges 370 extending along outer portions of their tabs, this is not meant to be limiting in any way, and commissures 345 can be formed in any suitable configuration, without exceeding the scope of the disclosure.

Frame 320 is constructed to form an enclosure 325. In one further example, frame 320 is generally cylindrical shaped. Inner skirt 350 is positioned within enclosure 325 and outer face 351 of inner skirt 350 is juxtaposed with frame 320 such that inner face 352 faces enclosure 325. Outer skirt 360 is positioned outside of enclosure 325. The inner face of outer skirt 360 is juxtaposed with frame 320 such that outer face 361 faces away from frame 320. Each of inner skirt 350 and outer skirt 360 is secured to frame 320.

Inflatable medical balloon 40 is positioned within enclosure 325, such that sensor position member 50 is positioned between inflatable medical balloon 40 and prosthetic valve 310. In one example, sensor position member 50 is secured by inflatable medical balloon 40 and prosthetic valve 310, i.e. sensor position member 50 is held in place due to inflatable medical balloon 40 pushing against prosthetic valve 310. Catheter 70 is positioned within delivery shaft 311. In an example where outer shaft 312 is provided, delivery shaft 311 is positioned within outer shaft 312. Catheter 70, delivery shaft 311 and outer shaft 312 extend from handle 20.

FIGS. 4A-4B illustrate various deployment stages of medical balloon sensing assembly 300, in accordance with certain examples. In operation, as described above in relation to medical balloon sensing assembly 10, a user holds handle 20 and maneuvers delivery shaft 311 such that inflatable medical balloon 40 and prosthetic valve 310 are inserted into a patient lumen 95 and positioned at a desired implantation site. In one example, during delivery, prosthetic valve 310 is secured within delivery shaft 311 in a crimped state. In another example, delivery shaft 311 is maneuvered along with outer shaft 312.

In one example, during delivery of prosthetic valve 310, handle 20 is maneuvered by an operator (e.g., a clinician or a surgeon) to axially advance or retract components of medical balloon sensing assembly 300, such as nosecone shaft 80, delivery shaft 311 and/or outer shaft 312, through the patient's vasculature. In another example, handle 20 comprises one or more operating interfaces, such as steerable or rotatable adjustment knobs, levers, sliders, buttons (not shown) and other actuating mechanisms, which are operatively connected to different components of medical balloon sensing assembly 300 and configured to produce axial movement of medical balloon sensing assembly 300 in the proximal and distal directions. Particularly, in an example where rotatable knob 315 is provided, and operatively coupled to a pull wire, rotating rotatable knob 315 increases or decreases the tension in the pull wire, thereby adjusting the curvature of distal end portion 313 of delivery shaft 311 and therefore the distal end portion 302 of medical balloon sensing assembly 300. As illustrated, distal end portion 302 of medical balloon sensing assembly 300 comprises inflatable medical balloon 40 and prosthetic valve 310.

Medical balloon sensing assembly 300 can be utilized, for example, to deliver prosthetic valve 310 for mounting against the aortic annulus, the mitral annulus or any other native annulus. In one example (not shown), a pusher is further provided, the pusher arranged to push prosthetic valve 310 out of delivery shaft 311. In one further example, prosthetic valve 310 is crimped over a portion of catheter 70, proximal to inflatable medical balloon 40, or such that only a distal portion of prosthetic valve 310 is crimped over inflatable medical balloon 40, so as to reduce the overall profile in a crimped state during delivery. In such an example, the pusher can be utilized to push the balloon to extend entirely over the balloon upon reaching the site of implantation, prior to balloon inflation.

As illustrated in FIG. 4A, inflatable medical balloon 40 is initially in a deflated state and prosthetic valve 310 is in a compressed state. In one example, prosthetic valve 310 and inflatable medical balloon 40 are initially retained within outer shaft 312, and once inflatable medical balloon 40 and prosthetic valve 310 are positioned at the desired implantation site they are pushed out of outer shaft 312 and/or outer shaft 312 is pulled back therefrom. Particularly, in one example, a distal end portion of outer shaft 312 extends over prosthetic valve 310 and contacts nosecone 80 in a delivery configuration (not shown) of medical balloon sensing assembly 300. Thus, in such an example, the distal end portion of outer shaft 312 serves as a delivery capsule that contains, or houses, prosthetic valve 310 in a radially compressed or crimped configuration for delivery through the patient's vasculature. In one example, outer shaft 312 and delivery shaft 311 are configured to be axially movable relative to each other, such that a proximally oriented movement of outer shaft 312 relative to delivery shaft 311, or a distally oriented movement of delivery shaft 311 relative to outer shaft 312, removes prosthetic valve 310 and inflatable medical balloon 40 from the confines of outer shaft 312. In an alternative example, prosthetic valve 310 is not housed within outer shaft 312 during delivery and in such an example outer shaft 312 is optionally not provided.

After positioning inflatable medical balloon 40 at the desired implantation site, an inflation fluid (not shown) is injected into inflatable medical balloon 40, as described above. As the inflation fluid fills inflatable medical balloon 40, inflatable medical balloon 40 is inflated, thereby causing prosthetic valve 310 to expand into an expanded state. In one example, the expanded state includes a range of diameters to which prosthetic valve 310 can expand, between the compressed state and a maximal diameter reached at a fully expanded state. Thus, a plurality of partially expanded states may relate to any expansion diameter between a radially compressed, or crimped state, and a maximally expanded state.

As described above, sensor position member 50 is juxtaposed with outer face 43 of inflatable medical balloon 40 such that the inflation of inflatable medical balloon 40 causes sensor position member 50 to expand. As further described above, in one example, sensor data unit 90 determines a diameter indication of inflatable medical balloon 40, responsive to an output of one or more diameter sensors 60. In one further example, as described above, the diameter indication is determined at predetermined intervals during inflation and in another further example is determined continuously during inflation. As described above, in one example, upon detection that the diameter is no longer increasing, i.e. that prosthetic valve 310 is pushing against the walls of patient lumen 95, sensor data unit 90 outputs a signal indicating that the diameter is no longer increasing. In such a case, as described above, it may be desired to cease the injection of inflation fluid into inflatable medical balloon 40. In one example, as will be described below, sensor data unit 90 further controls a pump to cease the injection of inflation fluid into inflatable medical balloon 40.

As further described above, in one example at least one sensor 60 comprises one or more force and/or pressure sensors 60. As prosthetic valve 310 pushes against the walls of patient lumen 95, pressure is applied between prosthetic valve 310 and sensors 60. Responsive to the output of sensors 60, sensor data unit 90 determines the pressure applied between prosthetic valve 310 and sensors 60, which translates to the pressure applied between prosthetic valve 310 and the walls of patient lumen 95.

As described above, in one example sensor data unit 90 determines a map of the forces applied between prosthetic valve 310 and inflatable medical balloon 40, responsive to the output of sensors 60. In another example, responsive to the outputs of sensors 60, sensor data unit 90 determines locations of increased force and/or pressure, as described above. In one further example, sensor data unit 90 compares the locations of increased force/pressure to predetermined locations on prosthetic valve 310. Responsive to an outcome of the comparison indicating that one or more of the predetermined locations on prosthetic valve 310 exhibit force/pressure above a respective predetermined threshold, or exhibit increased force/pressure in relation to an average force/pressure, sensor data unit 90 outputs a respective signal indicative of the increase force/pressure, optionally being displayed on display 92. For example, if increased pressure is applied to the location of one of leaflets 340, or commissures 345, this can disrupt the abilities of leaflets 340 to function properly as a replacement for the cardiac valve. Advantageously, such an indication can allow the surgeon to move prosthetic valve 310 to a more suitable location.

Although medical balloon sensing assembly 300 is described herein as comprising sensor position member 50, this is not meant to be limiting in any way. In another example (not shown), medical balloon sensing assembly 300 comprises one or more sensor position members 160, as described above in relation to medical balloon sensing assembly 150. In another example (not shown), sensors 60 are positioned on outer face 43 of inflatable medical balloon 40. In another example (not shown), medical balloon sensing assembly 300 comprises an inflatable medical balloon 200, as described above. In one further example (not shown), sensor position member 50 is not provided since sensors 60 are embedded within inflatable medical balloon 40, as described above.

Although medical balloon sensing assembly 300 is described herein in an example where sensors 60 are only positioned between catheter 70 and prosthetic valve 310, this is not meant to be limiting in any way. In another example (not shown), some of sensors 60 are positioned on prosthetic valve 310. In one further example (not shown), one or more force sensors 60 are positioned on prosthetic valve 310 and one or more diameter sensors 60 are positioned on sensor position member 50, on sensor position members 160, on inflatable medical balloon 40, or embedded in inflatable medical balloon 200. In another further example (not shown), one or more diameter sensors 60 are positioned on prosthetic valve 310 and one or more force sensors 60 are positioned on sensor position member 50, on sensor position members 160, on inflatable medical balloon 40, or embedded in inflatable medical balloon 200.

Once prosthetic valve 310 is fully expanded, inflatable medical balloon 200 is deflated and removed along with the remainder of medical balloon sensing assembly 10. Because frame 320 is preferably plastically-deformable, it substantially retains its expanded state.

Although medical balloon sensing assembly 300 is described herein where sensors 60 are secured to one or more sensor position members 50, this is not meant to be limiting in any way. In another example (not shown), sensors 60 are secured to one or more sensor position members 160, as described above in relation to medical balloon sensing assembly 150. In another example (not shown), sensors 60 are positioned within respective depressions 210, as described above in relation to inflatable medical balloon 200. In another example (not shown), sensors 60 are positioned directly on outer face 43 and/or inner face 44 of inflatable medical balloon 40.

Thus, as described above in relation to FIGS. 1A-4C, sensors 60 are provided and juxtaposed with a surface of an inflatable medical balloon. In one example, sensors 60 are secured to one or more sensor position members arranged to contact an outer face of the surface of the inflatable medical balloon. In another example, sensors 60 are positioned within respective depressions in the surface of the inflatable medical balloon. In another example, sensors 60 are positioned on an outer face and/or inner face of the surface of the inflatable medical balloon.

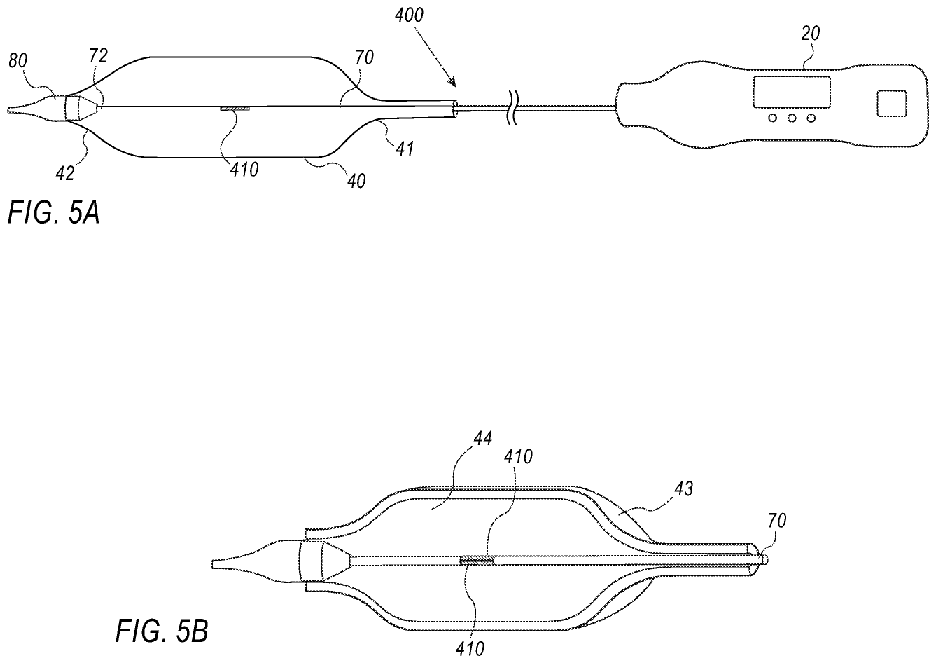

FIG. 5A illustrates a high-level perspective view of a medical balloon sensing assembly 400 and FIG. 5B illustrates a high-level perspective cut-away view of a portion of medical balloon sensing assembly 400, in accordance with certain examples. FIGS. 5A-5B are described together. Medical balloon sensing assembly 400 comprises: a handle 20, exhibiting a proximal end 21 and a distal end 22; an inflatable medical balloon 40, extending from a proximal end 41 to a distal end 42, and exhibiting an outer face 43 and an inner face 44 opposing outer face 43; a catheter 70, extending towards a distal end 72; and one or more ultrasound transducers 410. In one example, medical balloon sensing assembly 400 further comprises: a nosecone 80; a sensor data unit 90; one or more visual or auditory informative elements configured to provide visual or auditory information and/or feedback to a user or operator of medical balloon sensing assembly 10, such as a display 92, LED lights, speakers (not shown) and the like.

As described above in relation to medical balloon sensing assembly 10, catheter 70 extends from handle 20 towards distal end 72. Inflatable medical balloon 40 is secured to catheter 70 and catheter 70 extends into inflatable medical balloon 40, as known to those skilled in the art. In another example, nosecone 80 is secured to distal end 72 of catheter 70 and/or to distal end 42 of inflatable medical balloon 40.

One or more ultrasound transducers 410 are positioned on catheter 70, within inflatable medical balloon 40, such that each ultrasound transducer faces inner face 44 of inflatable medical balloon 40. In one example, as illustrated in FIG. 5B, at least two ultrasound transducers 410 are provided, the orientation of a first ultrasound transducer 410 opposing the orientation of the second ultrasound transducer 410. Particularly, in one example, where each ultrasound transducer 410 exhibits a 180-degree scanning range, the first ultrasound transducer 410 is oriented to cover a predetermined 180 degrees of the circumference of inflatable medical balloon 40 and the second ultrasound transducer 410 is oriented to cover the other 180 degrees of the circumference of inflatable medical balloon 40. In another example (not shown), more than two ultrasound transducers are provided. For example, an array of ultrasound transducers can span circumferentially (e.g., around catheter 70), configured to cover 360-degree scanning.

As described above in relation to medical balloon sensing assembly 10, in one example (not shown), a guidewire extends through a central lumen of catheter 70 and an inner lumen of nosecone 80, so that catheter 70 can be advanced over the guidewire through the patient's vasculature. In one example, each ultrasound transducer 410 is in communication with sensor data unit 90. In one further example (not shown), each ultrasound transducer 410 is in communication with sensor data unit 90 via a respective communication channel. In one yet further example, each communication channel is configured to allow: electrical communication via a conductive material, such as a wire; and/or optical communication, e.g. via an optical fiber. In another further example, each ultrasound transducer 410 is in wireless communication with sensor data unit 90. In another example (not shown), each ultrasound transducer 410 is in communication with an external computing device. In one example, each ultrasound transducer is operated by sensor data unit 90, as described above in relation to sensors 60. In another example, each ultrasound transducer 410 comprises dedicated circuitry for operation and sensor data unit 90 receives the measured data from ultrasound transducer 410.

The operation of medical balloon sensing assembly 400 is in all respects similar to the operation of medical balloon sensing assembly 10, with the exception that a diameter indication is measured by one or more ultrasound transducers 410. Particularly, upon inflation of inflatable medical balloon 40, each ultrasound transducer 410 generates ultrasound waves and measures the waves reflected from inner face 44 of inflatable medical balloon 40. Responsive to the measured waves, sensor data unit 90 determines a diameter indication of inflatable medical balloon 40.

In one example, the diameter indication comprises a radial diameter of inflatable medical balloon 40. In another example, the diameter indication comprises the distance between the respective ultrasound transducer 410 and inner face 44 of inflatable medical balloon 40, i.e. a radial radius of inflatable medical balloon 40. In one example, the radial diameter comprises a sum of: the distance between a first ultrasound transducer 410 and inner face 44 of inflatable medical balloon 40; and the distance between a second ultrasound transducer 410 and inner face 44 of inflatable medical balloon 40, where the orientation of the second ultrasound transducer 410 generally opposes the orientation of the first ultrasound transducer 410. Catheter 70 may not be exactly in the center of inflatable medical balloon 40, thus the distance between one ultrasound transducer 410 and inner face 44 may not give an exact radius of inflatable medical balloon 40. Advantageously, using the sum of the measurements of two ultrasound transducers 410 gives a more accurate measurement of the diameter of inflatable medical balloon 40.

In another example, the diameter indication comprises a surface topography of inflatable medical balloon 40 and/or patient lumen 95, as described above in relation to flex sensors 60. In one further example, responsive to the determined surface topography, sensor data unit 90 identifies the presence and severity of calcifications, as described above. In one example, the diameter indication comprises a protrusion depth of one or more protrusions extending into the inflatable medical balloon, optionally determined responsive to the determined surface topography. The term “protrusion depth”, as used herein, means the depth to which the protrusion protrudes into the inflatable medical balloon. The protrusion depth of a calcification can indicate the severity of damage that can be caused by the calcification. In another example, the diameter indication comprises a height-diameter aspect ratio of a protrusion extending into the inflatable medical balloon, optionally determined responsive to the determined surface topography. As further described above, the height-diameter aspect ratio of a calcification can indicate the severity of damage that can be caused by the calcification.

In one example, as described above, responsive to the determined diameter indication, sensor data unit 90 outputs an indication of the maximum diameter allowed for expansion in patient lumen 95. For example, inflatable medical balloon 40 can be used to determine the size of patient lumen 95 for purposes of determining the target expansion diameter of a prosthetic valve. Thus, sensor data unit 90 measures the diameter of patient lumen 95 when inflatable medical balloon 40 is inflated, thereby identifying the expansion diameter for a prosthetic valve.

In one example, responsive to the output one or more ultrasound transducers 410, sensor data unit 90 determines an indication of the density of various points and/or areas of patient lumen 95. Particularly, the reflection coefficient of the tissue is dependent on its density. Thus, responsive to the intensity of the ultrasound signal returning to the one or more ultrasound transducers 410, sensor data unit 90 determines the indication of the tissue density. In one example, the indication of the density comprises the density value of the tissue at the respective point and/or area. In another example, the indication of the density of the tissue comprises the extent of calcification at the respective point and/or area. In another example, the indication of the density of the tissue comprises an indication of the presence of a calcification at the respective point and/or area.

In one example, the determination of an indication of density is performed in addition to the determination of a diameter indication. In one further example, where the diameter indication comprises a surface topography of inflatable medical balloon 40 and/or patient lumen 95, sensor data unit 90 generates a 4-dimensional (4D) map of the surface of patient lumen 95. Particularly, the 4D map comprises: the 3 dimensions of the surface topography of patient lumen 95; and a fourth dimension comprising information regarding the density and/or type of material of the surface. In one example, the 4D map is output to an external unit. In another example, the 4D map is displayed on a user display. In one further example, the 4D map is color or shade coded, such that calcifications and tissue are displayed in different colors/shades. In another further example, the 4D is color or shade coded, such that the tissue density of patient lumen 95 is indicated by the color/shade.

In one example (not shown), additional sensors are provided to measure the force and/or pressure applied to inflatable medical balloon 40. As described above, the additional sensors can be positioned on outer face 43 of inflatable medical balloon 40, embedded in the material of inflatable medical balloon 40 and/or secured to one or more sensor position members. In another example (not shown), medical balloon sensing assembly 400 further comprises a prosthetic implant, or other balloon expandable device, with inflatable medical balloon 40 positioned within the balloon expandable device, as known to those skilled in the art.

In one example (not shown), a plurality of markers exhibiting a high acoustic reflection coefficient are positioned on outer surface 43 and/or inner surface 44 of inflatable medical balloon 40, as described below in relation to imaging markers 960. In such an example, the markers increase the sensitivity of the ultrasound measurements. In another example, inflatable medical balloon 40, or a portion thereof, is coated with a predetermined coating exhibiting a high acoustic reflection coefficient. In such an example, the coating increases the sensitivity of the ultrasound measurements. In another example (not shown), where a prosthetic valve surrounds inflatable medical balloon 40, sensor data unit 90 determines the diameter of the prosthetic valve responsive to the reflection of the ultrasound waves therefrom. In one further example, a plurality of markers exhibiting a high acoustic reflection coefficient are positioned on the prosthetic valve to increase the sensitivity of the ultrasound measurements.

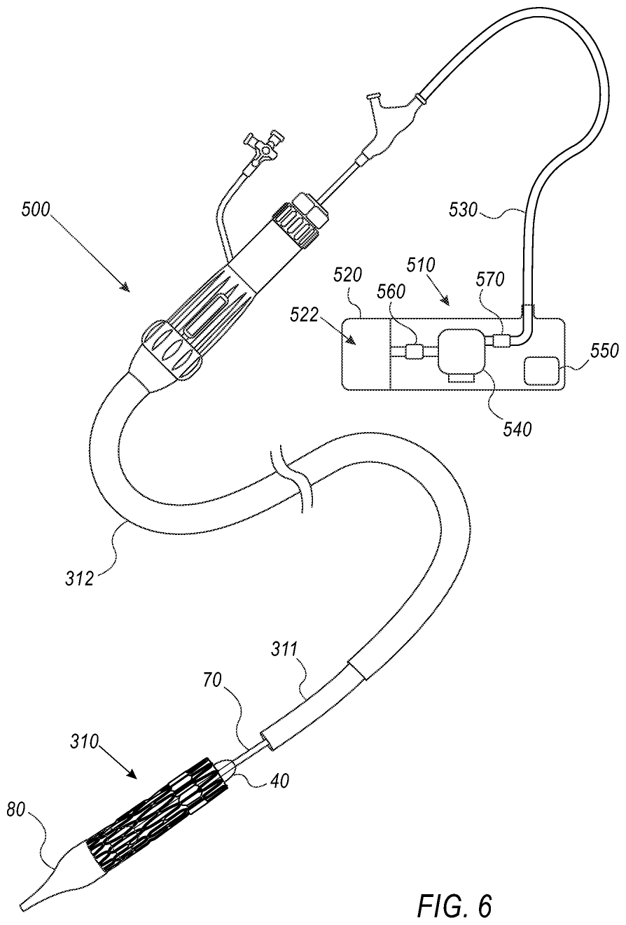

FIG. 6 illustrates a high-level perspective view of a medical balloon sensing assembly 500, in accordance with certain examples. Medical balloon sensing assembly 500 is in all respects similar to medical balloon sensing assembly 10, with the addition of an inflation fluid system 510. Inflation fluid system 510 comprises: a reservoir 520 containing a predetermined volume of inflation fluid 522; a fluid flow channel 530; a pump 540; an optional sensor data unit 550; an optional flow meter 560; and an optional pressure sensor 570. For simplicity, sensor position member 50 and sensors 60 are not shown.

In one example, optional sensor data unit 550 comprises a central processing unit (CPU), a microprocessor, a microcomputer, a programmable logic controller, an application-specific integrated circuit (ASIC) and/or a field-programmable gate array (FPGA), without limitation. In one example, optional sensor data unit 550 is implemented as part of sensor data unit 90 (not shown). In another example, optional sensor data unit 550 is a standalone unit.

Fluid flow channel 530 is in fluid communication with catheter 70. In one example, fluid flow channel 530 is coupled to catheter 70. In another example, fluid flow channel is implemented as catheter 70. In another example, fluid flow channel 530 extends through, and along, catheter 70. As described above, inflation fluid 522 comprises saline and/or sterile water, without limitation. As further described above, in one example inflation fluid 522 exhibits a temperature less than 31 degrees Celsius, optionally less than 21 degrees Celsius. The term “fluid communication”, as used herein, means that fluid can flow between components in fluid communication with each other. The fluid communication can be accomplished via a direct connection between openings of the respective components or via additional components connected therebetween.

In one example, pump 540 is in fluid communication with both reservoir 520 and fluid flow channel 530. In one example, a control input of pump 540 is in communication with optional sensor data unit 550. In one example, optional flow meter 560 is coupled between pump 540 and reservoir 520. Although optional pressure sensor 570 is illustrated as being near pump 540, this is not meant to be limiting in any way, and optional pressure sensor 570 can be positioned anywhere along the fluid path, including within inflatable medical balloon 40. In one example, optional flow meter 560 and/or optional pressure sensor 570 is in communication with optional sensor data unit 550.

In operation, pump 540 pumps inflation fluid 522 through catheter 70 into inflatable medical balloon 40, thereby inflating inflatable medical balloon 40. In one example, optional sensor data unit 550 controls pump 540 to initiate the pumping. In one example, optional sensor data unit 550 controls pump 540 to adjust the flow of inflation fluid 522. In one further example, adjustment of the flow of inflation fluid 522 includes adjustment of the flow rate and/or the amount of inflation fluid 522 that flows into inflatable medical balloon 40. In one example, the flow adjustment is responsive to: a user input, such as an input indicating the amount of inflation fluid 522 to be injected into inflatable medical balloon 40; optional flow meter 560 and/or optional pressure sensor 570, such that the flow of inflation fluid 522 remains within predetermined parameters; and/or outputs of sensors 60. In one example, optional sensor data unit 550 is further arranged to control pump 540 to reverse the flow of inflation fluid 522, thereby removing some, or all, of inflation fluid 522 from inflatable medical balloon 40.

As described above in relation to medical balloon sensing assembly 10, in one example sensor data unit 90, and/or optional sensor data unit 550, determines a diameter indication and/or force/pressure measurements of inflatable medical balloon 40. As described above, responsive to the determined diameter indication and/or measurements, the flow of inflation fluid 522 is adjusted. In one example, the flow of inflation fluid is adjusted by controlling pump 540. As described above, the flow of inflation fluid 522 can be adjusted to: adjust the diameter of inflatable medical balloon 40, and/or adjust the pressure applied by inflatable medical balloon 40. In one example, for deflation of inflatable medical balloon 40, optional sensor data unit 550 controls pump 540 to reverse the flow of inflation fluid 522, thereby emptying inflatable medical balloon 40 of inflation fluid 522.

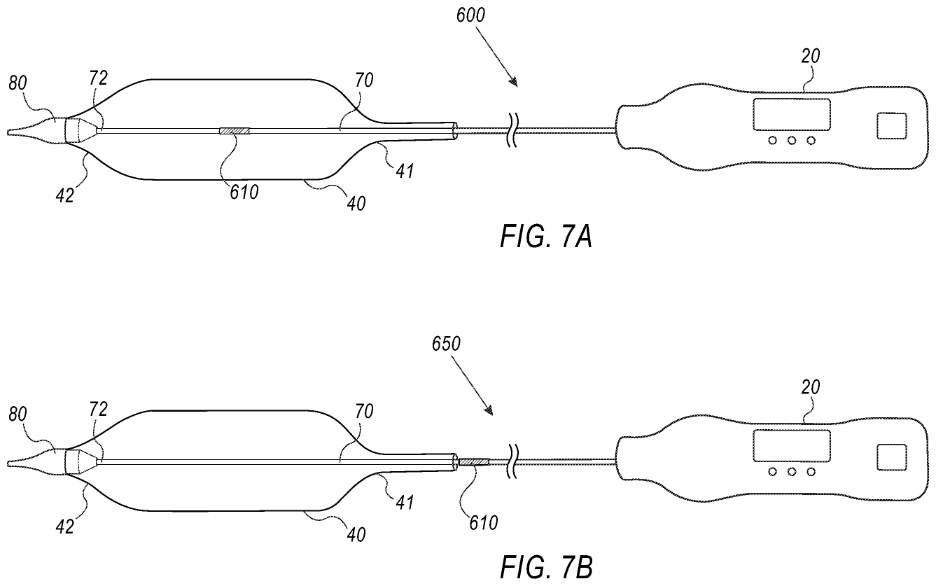

FIG. 7A illustrates a high-level side view of a medical balloon sensing assembly 600, in accordance with certain examples. Medical balloon sensing assembly 600 is in all respects similar to medical balloon sensing assembly 400 of FIGS. 5A-5C, with the exception that ultrasound transducers 410 are replaced with at least one thermal camera 610. In one example, at least one thermal camera 610 comprises at least one infrared camera, such as a thermographic camera. At least one thermal camera 610 is secured to catheter 70, within inflatable medical balloon 40. In one example, at least one thermal camera 610 is sized and shaped so as to provide thermal imaging along the entire circumference of inflatable medical balloon 40. Thermal camera 610 is illustrated as being disposed directly on catheter 70, however this is not meant to be limiting in any way. In another example, thermal camera 610 is secured to catheter 70, yet positioned away from catheter 70 towards inner face 44 of inflatable medical balloon 40. In one example, at least one thermal camera 610 is in communication with sensor data unit 90 and/or an external user display (not shown). Thermal camera 610 is illustrated as being positioned in the center of catheter 70, however this is not meant to be limiting in any way.

FIG. 7B illustrates a high-level side view of a medical balloon sensing assembly 650, in accordance with certain examples. Medical balloon sensing assembly 650 is in all respects similar to medical balloon sensing assembly 600, with the exception that at least one thermal camera 610 is secured to catheter 70 at a position proximal to inflatable medical balloon 40, i.e. between inflatable medical balloon 40 and handle 20.

The operation of medical balloon sensing assemblies 600 and 650 will be described together. Particularly, in operation, at least one thermal camera 610 generates a thermal image of the area surrounding inflatable medical balloon 40. As described above in relation to thermal sensors 60, the generated thermal image comprises the temperature at each of a plurality of predetermined locations juxtaposed with the inflatable medical balloon, i.e. the temperature at each pixel, or group of pixels, of the image. As further described above, in one example sensor data unit 90 determines, responsive to the generated thermal image, an indication of the rate of thermal dispersion between inflatable medical balloon 40 and the plurality of predetermined locations. In one further example, as described above, sensor data unit 90 outputs information regarding the indication of the rate of thermal dispersion. The operation of sensor data unit 90 in relation to at least one thermal camera 610 is in all respects similar to the operation of sensor data unit 90 in relation to at least one thermal sensor 60, and in the interest of brevity will not be further described.

Although the above has been described in relation to examples where at least one thermal camera 610 is secured to catheter 70, this is not meant to be limiting in any way. In another example (not shown), at least one thermal camera 610 is secured to nosecone 80 and/or outer face 43 of inflatable medical balloon 40.

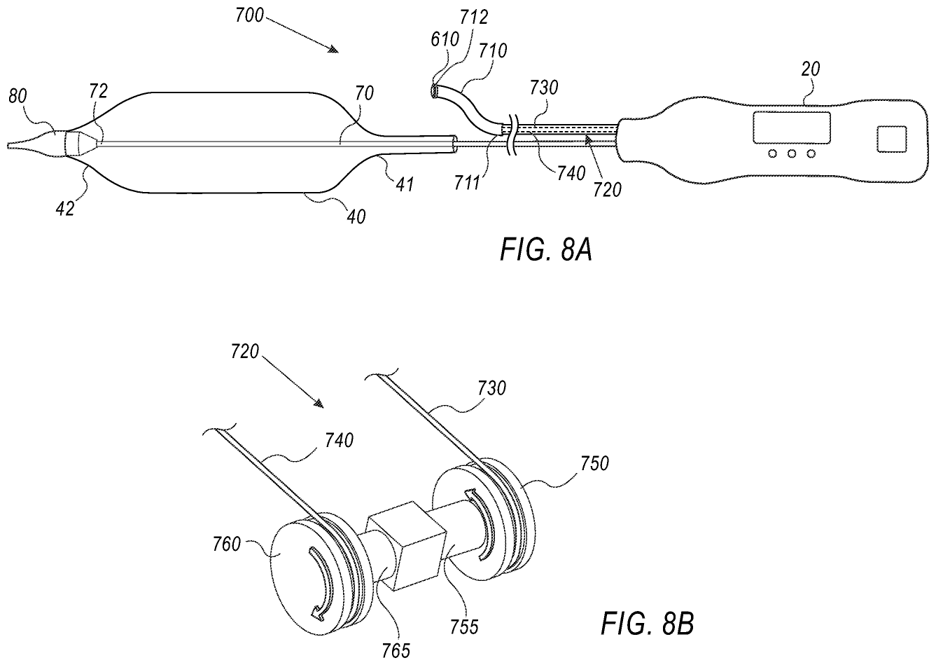

FIG. 8A illustrates a high-level side view of a medical balloon sensing assembly 700, in accordance with certain examples. Medical balloon sensing assembly 700 is in all respects similar to medical balloons sensing assemblies 600 and 650, with the exception that at least one thermal camera 610 is secured to a movable arm 710, movable arm 710 controlled by an arm translation assembly 720, illustrated in FIG. 8B. Movable arm 710 exhibits a proximal end 711 and a distal end 712, thermal camera 610 secured to distal end 712. In one illustrated example, arm translation assembly 720 comprises: a first wire 730; a second wire 740; a first pulley 750; a first motor 755; a second pulley 760; and a second motor 765. First wire 730 is wrapped around first pulley 750 and is secured to distal end 712 of movable arm 710, through proximal end 711. Second wire 740 is wrapped around second pulley 750 and is secured to distal end 712 of movable arm 710, through proximal end 711.

First motor 755 is in mechanical communication with first pulley 750 and is arranged to rotate first pulley 750 in a first rotational direction. Second motor 765 is in mechanical communication with second pulley 760 and is arranged to rotated second pulley 760 in a second rotation direction. In one example, the second rotational direction opposes the first rotational direction. First motor 755 and second motor 765 are each in electrical communication with sensor data unit 90. In one example, arm translation assembly 720 is situated within handle 20. In another example, first wire 730 and second wire 740 are positioned within a dedicated catheter.

In operation, sensor data unit 90 controls first motor 755 and second motor 765 to rotate first pulley 750 and second pulley 760 such that movable arm 710 is translated in a variety of directions, such that thermal camera 610 can image the area around the entire circumference of inflatable medical balloon 40. As described above, at least one thermal camera 610 generates a thermal image of the area surrounding inflatable medical balloon 40. As described above in relation to thermal sensors 60, the generated thermal image comprises the temperature at each of a plurality of predetermined locations juxtaposed with the inflatable medical balloon, i.e. the temperature at each pixel, or group of pixels, of the image. As further described above, in one example sensor data unit 90 determines, responsive to the generated thermal image, an indication of the rate of thermal dispersion between inflatable medical balloon 40 and the plurality of predetermined locations. In one further example, as described above, sensor data unit 90 outputs information regarding the indication of the rate of thermal dispersion. The operation of sensor data unit 90 in relation to at least one thermal camera 610 is in all respects similar to the operation of sensor data unit 90 in relation to at least one thermal sensor 60, and in the interest of brevity will not be further described.

Although the above has been described in relation to a particular example of arm translation assembly 720, this is not meant to be limiting in any way, and any assembly arranged to provide movement of at least one thermal cameral 610.

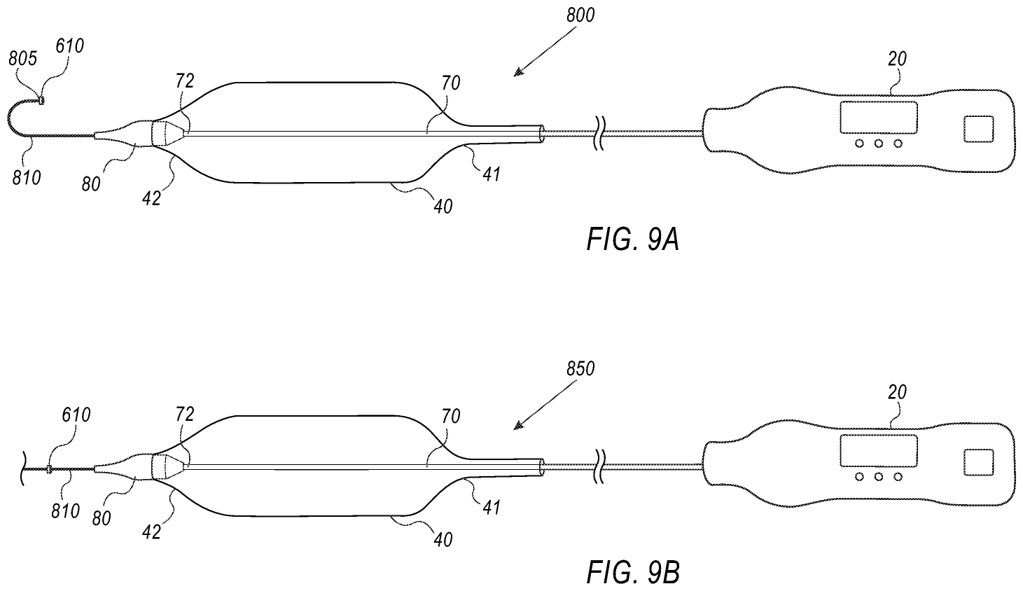

FIG. 9A illustrates a high-level side view of a medical balloon sensing assembly 800, in accordance with certain examples. Medical balloon sensing assembly 800 is in all respects similar to medical balloon sensing assembly 600, with the exception that at least one thermal camera 610 is secured to a distal tip 805 of a guidewire 810. Guidewire 810 extends from handle 20, through inflatable medical balloon 40 and nosecone 80 towards distal tip 805 thereof. In one example, a distal portion of guidewire 810 is generally J shaped, such that distal tip 805 faces inflatable medical balloon 40. As a result, thermal camera 610 faces inflatable medical balloon 40.

FIG. 9B illustrates a high-level side view of a medical balloon sensing assembly 850, in accordance with certain examples. Medical balloon sensing assembly 850 is in all respects similar to medical balloons sensing assembly 800, with the exception that at least one thermal camera 610 is secured to guidewire 810 at a position distal to inflatable medical balloon 40 and proximal to distal tip 805 (not shown). In another example (not shown), at least one thermal camera 610 is secured to nosecone 80. The operation of medical balloon sensing assemblies 800 and 850 will be described together.

Particularly, in operation, guidewire 810 can be translated proximally and distally to position thermal cameral 610 as desired. As described above, at least one thermal camera 610 generates a thermal image of the area surrounding inflatable medical balloon 40. As described above in relation to thermal sensors 60, the generated thermal image comprises the temperature at each of a plurality of predetermined locations juxtaposed with the inflatable medical balloon, i.e. the temperature at each pixel, or group of pixels, of the image. As further described above, in one example sensor data unit 90 determines, responsive to the generated thermal image, an indication of the rate of thermal dispersion between inflatable medical balloon 40 and the plurality of predetermined locations. In one further example, as described above, sensor data unit 90 outputs information regarding the indication of the rate of thermal dispersion.

The operation of sensor data unit 90 in relation to at least one thermal camera 610 is in all respects similar to the operation of sensor data unit 90 in relation to at least one thermal sensor 60, and in the interest of brevity will not be further described. Although medical balloon sensing assemblies 600, 650, 700, 800 and 850 are described in relation to a thermal camera 610, this is not meant to be limiting in any way. In another example, thermal camera 610 is replaced with one or more optical sensors, such as one or more optical cameras and/or lasers. In one further example, the one or more optical sensors are arranged to image the inner walls of the patient lumen. In another further example, a plurality of markers are positioned on inflatable medical balloon 40 and the one or more optical sensors are arranged to image the markers. For example, in one example inflatable medical balloon 40 is at least partially optically transparent such that the one or more optical sensors have a line of sight with the markers when inflatable medical balloon 40 is expanded and in contact with the walls of the patient lumen. In such an example, sensor data unit 90 maps the inner surface of the patient lumen, responsive to the output of the one or more optical sensors.

In one example, the markers are arranged in a lined or mesh configuration, thereby providing greater accuracy for surface mapping. The surface mapping can be performed using any suitable method. In one example, the method comprises: determining the distance between the different markers and/or between each marker and one or more predetermined reference points; responsive to the determined distances, defining the locations of predetermined points of the surface map; and interpolating the topography of the surface between adjacent points responsive to the determined distances.

In one example, the generation of the three-dimensional surface mapping of the patient lumen is performed using similar techniques to those employed in other motion capture processes (e.g., for video games or movies with computer-generated imagery). In one further example, the image processing differs from other motion capture processes due to the use of at least one moveable camera (e.g., in contrast to the multiple static cameras that are typically used on a motion capture stage to capture the same points as those points move in other motion capture processes). For example, a camera (e.g., mounted in a head of a fluoroscopy system) is rotated to capture points (e.g., markers on a balloon) at different angles.

In one example, specific markers may be put on the balloon and/or valve frame, and by rotating the head, these points can be seen from different angles. In another example, the head and/or camera is rotated multiple times to capture images from multiple perspectives and to track corresponding changes in the positions of the points. Using the images (e.g., processing the images in a similar manner to the above-referenced other motion capture techniques), the position of the markers in 3D space are in one example recreated (e.g., based on inputting the tracked changes in the positions of the points, the known relative positions of the points to one another, and the known positions/angles of the camera to a stereometric or other correlation model). In another example, the resolution of the tracking is increased by increasing the number of points that are tracked.

In one example, where multiple cameras are provided, each or a subset of the cameras face in different directions from one another (e.g., such that the cameras collectively image all or a majority portion of the surface of inflatable medical balloon 40). In another example, one or more of the camera(s) are fixed on catheter 70.

In another example, as described above, one or more of the camera(s) are moveable, such that at least a portion of each moveable camera is controllable to be directed to image the balloon from different angles (e.g., controllable to spin or otherwise rotate/tilt/twist/etc. relative to the catheter shaft to provide different views).

In one example, one or more of the camera(s) may be mounted within the proximal and/or distal stops of the balloon catheter.

In another example, one or more fiber optic cameras are incorporated into the catheter shaft. Particularly, thermal camera 610 described above in relation to medical balloon sensing assemblies 600 and 650 is in one example replaced, or complemented by, one or more fiber optic cameras positioned on catheter 70. In one further example, one or more fiber optic cameras each include a lens mounted in inflatable medical balloon 40 and an optical fiber extending from the lens to a proximal end of catheter 70 where it can be coupled to an eye piece or controller for generating image(s). In such an example, the lenses are optionally in a fixed position and/or moveable, as described above for the catheter-mounted cameras.

In one example, the above-described cameras use visible light, infrared light and/or another suitable light. In another example, a lighting source, such as a light-emitting diode (LED), is mounted on catheter 70, within the proximal and/or distal stoppers, via a fiber optic cable, and/or in another location relative to the camera(s) in order to aid in visualization. In one example, a combination of LED color temperature and light frequency is used to visualize through blood and image tissues, such as blood vessel walls, valve leaflets and calcifications.

In one example, where the markers are used in combination with an external imaging system, such as a fluoroscopy system, the external imaging system images the patient (targeting an area of the patient in which the inflatable medical balloon is being inserted/expanded) from multiple angles in order to detect the markers. For example, as the inflatable medical balloon inflates, the system generates a three-dimensional image of the balloon and/or the surface of the patient lumen based on a detection of the markers as imaged from different angles around a patient and a determination of locations of the markers over time.

Although medical balloon sensing assemblies 600, 650, 700, 800 and 850 are described in relation to an example where sensor data unit 90 determines indications of thermal diffusion, responsive to the output of thermal camera 610, this is not meant to be limiting in any way. In another example, as described above, infrared markers are positioned on inflatable medical balloon 40 and one or more thermal cameras 610 identify the positions of the infrared markers. In such an example, sensor data unit 90 maps the inner surface of the patient lumen, responsive to the output of the one or more thermal cameras 610, as described above.

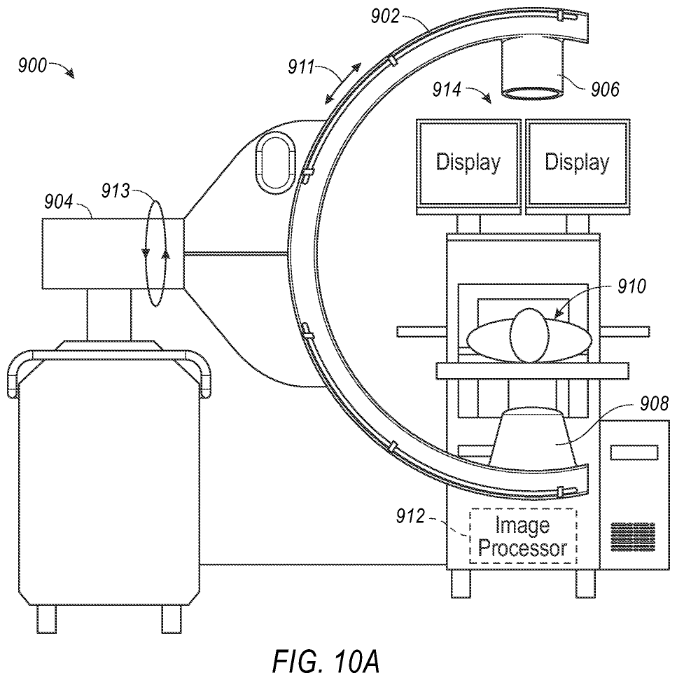

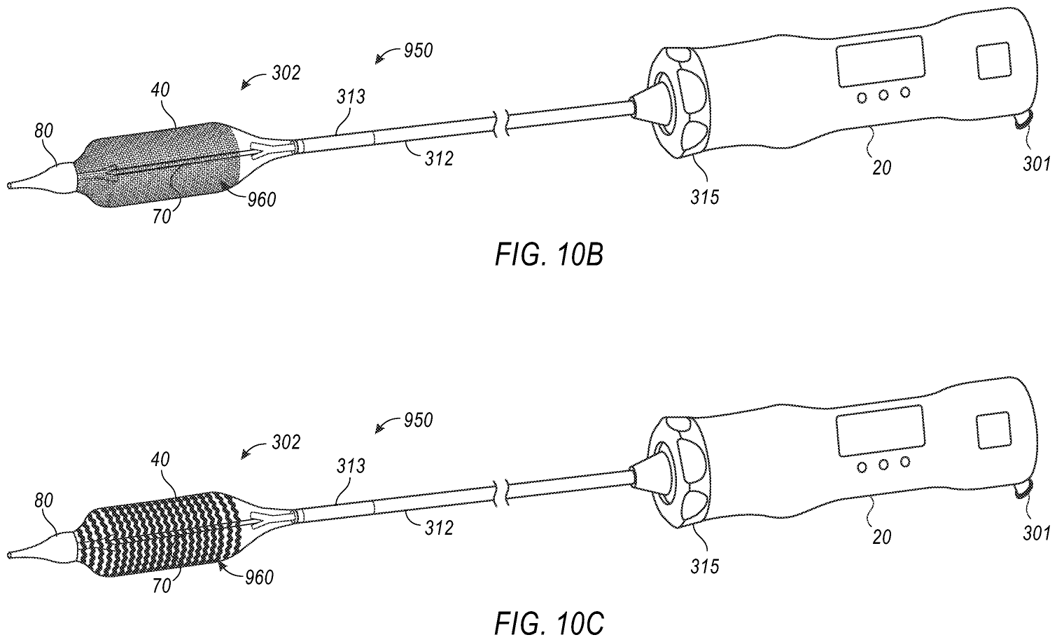

FIG. 10A illustrates a high-level view of an imaging system 900, in accordance with certain examples. Imaging system 900 comprises: a C-arm 902 coupled to a base arm 904. C-arm 902 includes an image detector 906 (e.g., an image intensifier or other element for capturing x-rays and converting the captured x-rays to an image) and an x-ray emitter 908 (e.g., an x-ray tube configured to emit x-rays that penetrate a targeted patient, such as patient 910). In one example, image detector 906 and x-ray emitter 908 are components of a fluoroscopy imaging system, a computed tomography (CT) imaging system or any other suitable imaging system. In another example, C-arm 902 is configured to rotate or slide relative to base arm 904 in the directions indicated by arrows 911. Additionally, base arm 904 is in one example configured to rotate in the directions indicated by arrows 913, such that the rotation is propagated to C-arm 902. In this way, C-arm 902 can move in order to image patient 910 from multiple angles using combinations of tilt, swing, and swivel motions. The output from image detector 906 is in one example provided to a sensor data unit 912 in order to configure the detected images for display via a display device 914. In another example, the image data is provided to the display in real-time or near real-time in order to provide on-going monitoring of patient 910 (e.g., as the balloon is inserted and expanded within the patient).

Although the above has been described in relation to an imaging system 900, this is not meant to be limiting in any way, and patient 910 can be imaged with any suitable imaging system without exceeding the scope of the disclosure.

FIG. 10B illustrates a high-level side view of a first example of a medical balloon sensing assembly 950 and FIG. 10C illustrates a high-level side view of a second example of medical balloons sensing assembly 950. Medical balloon sensing assembly 950 is in all respects similar to medical balloon sensing assembly 10, with the exception that sensors 60 are replaced with imaging markers 960. As such, communication mediums 65 are also not provided. Imaging markers 960 can exhibit any suitable shape. In one example, as illustrated in FIG. 10B, imaging markers 960 are generally square shaped. In another example, as illustrated in FIG. 10C, each imaging marker 960 exhibits an elongated shape surrounding the circumference of inflatable medical balloon 40, such as a zig-zag pattern.

Sensor position member 50 is not illustrated for simplicity, however this is not meant to be limiting in any way. Although medical balloon sensing assembly 950 is described in relation to an example where sensors 60 are replaced with imaging markers 960, this is not meant to be limiting in any way. In another example, imaging markers 960 are provided in addition to sensors 60. In another example, sensors 60 serve as imaging markers 960. Particularly, in such an example, the radio opaqueness of each sensor 60 is detected during imaging.

Imaging markers 960 are juxtaposed with inflatable medical balloon 40. In one example (not shown), imaging markers 960 are positioned on a sensor position member 50, as described above in relation to medical balloon sensing assembly 10. In another example (not shown), imaging markers 960 are positioned on one or more position members 160, as described above in relation to medical balloon sensing assembly 150. In another example (not shown), imaging markers 960 are embedded within depressions of inflatable medical balloon 40, as described above in relation to inflatable medical balloon 200. In another example (not shown), imaging markers 960 are positioned within a woven structure of a fabric forming inflatable medical balloon 40, as described above. In another example, imaging markers 960 are positioned on the outer surface and/or inner surface of inflatable medical balloon 40.

In one example, imaging markers 960 are in the form of any suitable shape for imaging, including: dots (e.g., circular or ovular markers); parallel lines or stripes; grid or lattice formations; and/or other patterns that may be processed to generate a three-dimensional image of the surface of the patient lumen and/or the surface of inflatable medical balloon 40 using one or more imaging devices as described below.

In operation, inflatable medical balloon 40 of medical balloon sensing assembly 950 is delivered to a predetermined anatomical location, such as the location of a defective heart valve. Particularly, inflatable medical balloon 40 is inserted into a lumen of patient 910, as described above. Imaging system 900 then images inflatable medical balloon 40. Particularly, x-ray emitter 908 outputs radiation, which is then detected by image detector 906 after passing through patient 910. Although the imaging of inflatable medical balloon 40 of medical balloon sensing assembly 950 is described herein in relation to imaging system 900, this is not meant to be limiting in any way, and any suitable imaging system can be utilized without exceeding the scope of the disclosure.

Sensor data unit 912 receives the imaging data from image detector 906 and identifies imaging markers 960 within the image. Responsive to the identified imaging markers 960, sensor data unit 912 maps the surface of the patient lumen, as described above. In one example, as described above, the surface is mapped using techniques utilized in motion capture systems.

In one example (not shown), medical balloons sensing assembly 950 further comprises one or more thermal sensors 60 and/or one or more thermal cameras 610. In such an example, sensor data unit 90 and/or sensor data unit 912 further determines thermal diffusion properties of the mapped surface of the patient lumen, as described above. Particularly, as described above, the thermal dispersion rate of tissue is greater than that of calcifications. Thus, sensor data unit 90 and/or sensor data unit 912 determines an indication of the rate of thermal dispersion at various predetermined points along the mapped surface of the patient lumen. In one example, sensor data unit 90 and/or sensor data unit 912, further determines the extent of calcification of each of a plurality of areas on the mapped surface, responsive to the outputs of the one or more thermal sensors 60 and/or the one or more thermal cameras 610. In another example, sensor data unit 90 and/or sensor data unit 912 further determines the extent of calcification of each pixel, or group of pixels, on the mapped surface of the patient lumen.

Thus, a 4D map of the surface of the patient lumen is generated. Particularly, the 4D map comprises: the 3 dimensions of the surface topography of the patient lumen; and a fourth dimension comprising information regarding the rate of thermal dispersion and/or the type of material of the surface. In one example, the 4D map is output to an external unit. In another example, the 4D map is displayed on a user display. In one further example, the 4D map is color or shade coded, such that calcifications and tissue are displayed in different colors/shades. In another further example, the 4D is color or shade coded, such that the rate of thermal dispersion is indicated by the color/shade.

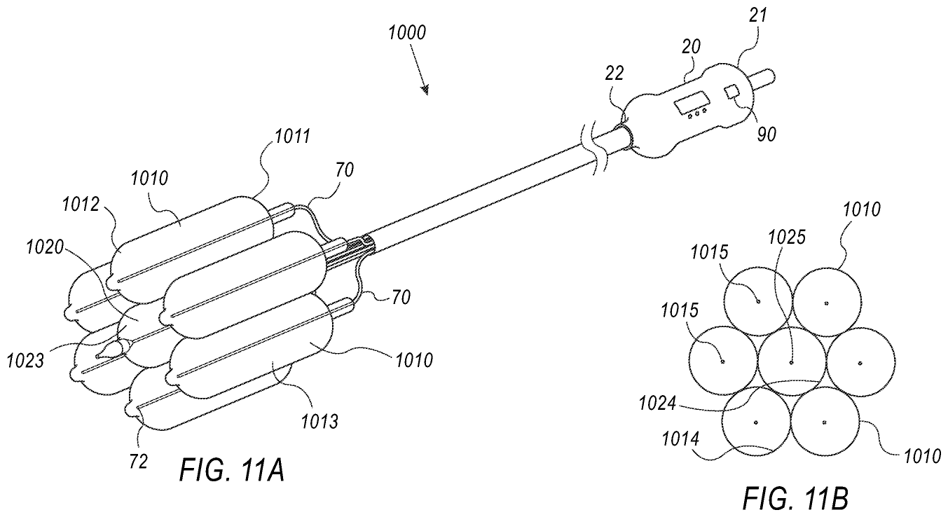

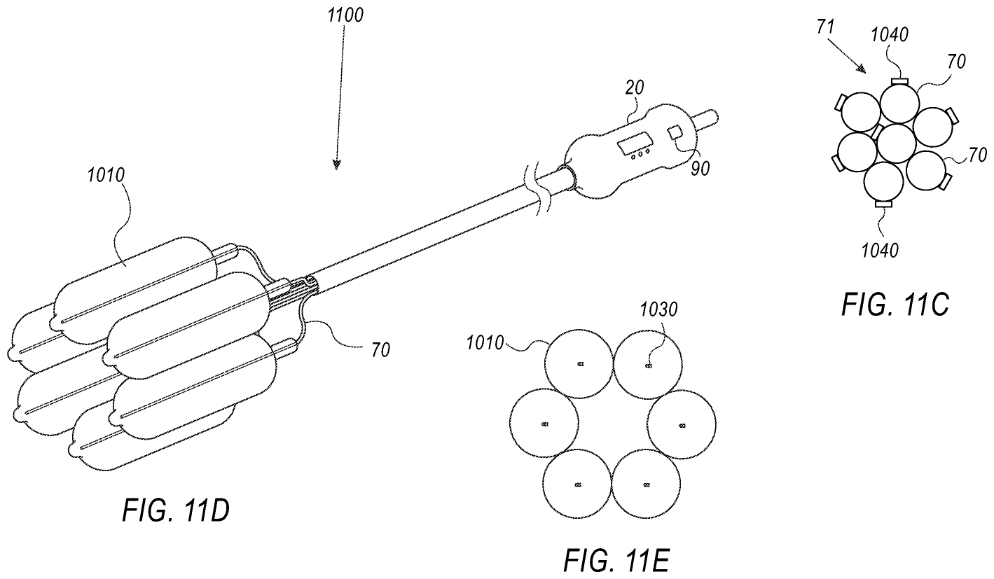

FIG. 11A illustrates a high-level perspective view of a medical balloon sensing assembly 1000, FIG. 11B illustrates a high-level cut-away view of a first portion of medical balloon sensing assembly 1000 and FIG. 11C illustrates a high-level cut-away view of a second portion of medical balloon sensing assembly 1000, in accordance with certain examples. Medical balloon sensing assembly 1000 comprises: a handle 20, exhibiting a proximal end 21 and a distal end 22; a plurality of inflatable medical balloons 1010, each extending from a respective proximal end 1011 to a respective distal 1012, and exhibiting an outer face 1013 and an inner face 1014; an inflatable medical balloon 1020, exhibiting an outer face 1023 and an inner face 1024; a plurality of catheters 70, each exhibiting a distal end 72; and a sensor data unit 90.

In one example, medical balloon sensing assembly 1000 further comprises a plurality of force sensors, such as pressure sensors 1030. In one further example, one or more respective pressure sensors 1030 are positioned within the inflation fluid flow of each inflatable medical balloon 1010, and optionally within the inflation fluid flow of inflatable medical balloon 1020. In one example, each pressure sensor 1030 is positioned within a respective inflatable medical balloon 1010 or within inflatable medical balloon 1020. In another example, each pressure sensor 1030 is positioned within a respective catheter 70. Each pressure sensor 1030 is in communication with sensor data unit 90, as described above in relation to sensors 60. In one example (not shown), a respective set of pressure sensors 1030 are juxtaposed with a surface of each inflatable medical balloon 1010. In one further example, each set of pressure sensors 1030 are positioned on an outer surface of the respective inflatable medical balloon 1010, on an inner surface of the respective inflatable medical balloon 1010, embedded within a surface of the respective inflatable medical balloon 1010 and/or secured to one or more sensor position members, as described above. In another example, each pressure sensor 1030 is secured to a respective catheter 70.

In another example, as illustrated in FIG. 11C, medical balloon sensing assembly 1000 further comprises a plurality of flow sensors 1040. Each flow sensor 1040 is juxtaposed with a respective catheter 70. FIG. 11C illustrates medical balloon sensing assembly 1000 in an example where flow sensors 1040 are each secured to an outer surface of a respective catheter 70, however this is not meant to be limiting in any way. In another example, each flow sensor 1040 is positioned within a respective catheter 70.

Preferably, each flow sensor 1040 is juxtaposed with a respective input port 71 of the respective catheter 70, either within the respective input port or outside of the respective input port 71. Similarly, in one example, each pressure sensor 1030 is juxtaposed with a respective input port 71 of the respective catheter 70, either within the respective input port 71 or outside of the respective input port 71. In one example, input port 71 of each catheter 70 is positioned within handle 20 and/or within an external pump device (not shown).

In one example (not shown), each catheter 70 is in fluid communication with a respective one of a plurality of pumps, as described above in relation to pump 540 of medical balloon sensing assembly 500.

In another example (not shown), medical balloon sensing assembly 1000 further comprises a plurality of diameter sensors, as described above in relation to diameter sensor/s 60 of medical balloon sensing assembly 10. In another example (not shown), medical balloon sensing assembly 1000 further comprises a plurality of thermal sensors, as described above in relation to thermal sensors 60 of medical balloons sensing assembly and/or thermal camera 610 of medical balloon sensing assemblies 600, 650, 700, 800 and 850. In another example (not shown), medical balloon sensing assembly 1000 further comprises one or more ultrasound transducers, as described above in relation to ultrasound transducers 410 of medical balloon sensing assembly 400.

In another example (not shown), medical balloon sensing assembly 1000 further comprises a plurality of flex sensors, as described above in relation to flex sensors 60 of medical balloon sensing assembly 10. In one example, a respective set of flex sensors are juxtaposed with a surface of each inflatable medical balloon 1010. In one further example, each set of flex sensors are positioned on an outer surface of the respective inflatable medical balloon 1010, on an inner surface of the respective inflatable medical balloon 1010, embedded within a surface of the respective inflatable medical balloon 1010 and/or secured to one or more sensor position members, as described above.

In one example, each inflatable medical balloon 1010, and further optionally each inflatable medical balloon 1020, has secured thereto a respective sensor position member 50 and/or at least one sensor position member 160, as described above in relation to medical balloon sensing assemblies 10 and 150. In another example, each inflatable medical balloon 1010, and further optionally each inflatable medical balloon 1020, exhibits one or more depressions, as described above in relation to inflatable medical balloon 200.

In another example, the configuration of each inflatable medical balloon 1010, and further optionally inflatable medical balloon 1020, is in all respects similar to the configuration of inflatable medical balloon 40 and/or 200, described above, with the respective sensors provided therewith.

Each catheter 70 extends from handle 20 into a respective inflatable medical balloon 1010 or inflatable medical balloon 1020, towards distal end 72 thereof. Thus, distal end 72 of each catheter 70 faces either inner face 1014 of a respective inflatable medical balloon 1010 or inner face 1024 of inflatable medical balloon 1020. In one example, handle 20 comprises a fluid port 301. In one further example, fluid port 301 is in fluid communication with a reservoir comprising an inflation fluid, as described above in relation to medical balloon sensing assembly 500. In another further example, fluid port 301 is shaped and dimensioned such that a syringe and/or manual pump can be connected thereto.

Inflatable medical balloons 1010 are radially arrayed about inflatable medical balloon 1020. Particularly, in one example each inflatable medical balloon 1010 exhibits a longitudinal axis 1015 extending through the respective proximal end 1011 and distal end 1012. Similarly, in such an example, inflatable medical balloon 1020 exhibits a longitudinal axis 1025 extending therethrough. In such an example, longitudinal axes 1015 are generally parallel to each other and to longitudinal axis 1025. Outer face 1013 of each inflatable medical balloon 1010 faces outer face 1013 of each of a pair of adjacent inflatable medical balloons 1010, and faces outer face 1023 of inflatable medical balloon 1020. In one further example, outer face 1013 of each inflatable medical balloon 1010 is in contact with outer face 1013 of each of the respective pair of adjacent inflatable medical balloons 1010. Advantageously, if inflatable medical balloons 1010 are in contact with each other, they provide structural support to one another. In another further example, outer face 1013 of each inflatable medical balloon 1010 is in contact with outer face 1023 of inflatable medical balloon 1020. Thus, advantageously, inflatable medical balloon 1020 provides structural support for inflatable medical balloon 1010.

In one example, an even number of inflatable medical balloons 1010 are provided. Medical balloon sensing assembly 1000 is illustrated as comprising 6 inflatable medical balloons 1010, however this is not meant to be limiting in any way, and any number of inflatable medical balloons 1010 can be provided without exceeding the scope of the disclosure. Advantageously, a greater number of inflatable medical balloons 1010 will mean that the force applied to each inflatable medical balloon 1010 is smaller. Further advantageously, a greater number of inflatable medical balloons 1010 provides a greater number of contact points with the tissue, thereby improving the resolution of the sensor measurements.

In operation, inflatable medical balloons 1010 and 1020 are inflated by injecting inflation fluid through the respective catheters 70. In one example, the inflation fluid is injected into each catheter 70 at the respective input port 71 thereof. As each inflatable medical balloon 1010 is inflated, one or more measurements are performed. As described above, the measurements include pressure measurements, thermal measurements, ultrasound measurements and/or flex measurements.

In one example, where each of a plurality of pressure sensors 1030, and/or each of a plurality of flow sensors 1040, is provided for each inflatable medical balloon 1010, each pressure sensor 1030 measures the total pressure applied to the respective inflatable medical balloon 1010 and each flow sensor 1040 measures the amount of flow of inflation fluid into the respective inflatable medical balloon 1010. In one further example, sensor data unit 90 determines, for each inflatable medical balloon 1010 and responsive to the output of the respective pressure sensor 1030 and/or the respective flow sensor 1040, when the respective inflatable medical balloon 1010 is fully inflated. Upon determining that a respective inflatable medical balloon 1010 is fully inflated, i.e. it is pressed against the wall of the patient lumen, sensor data unit 90 further determines the diameter of the respective inflatable medical balloon 1010 responsive to the output of the respective flow sensor 1040. In one further example, sensor data unit 90 further determines the diameter of the patient lumen responsive to the determined diameters of inflatable medical balloons 1010.

Advantageously, because each inflatable medical balloon 1010 is inflated via a separate catheter 70, the flow and/or pressure of the inflation fluid for each inflatable medical balloon 1010 can be sensed within handle 20, or within an external pumping system, without the need for placement of sensors within inflatable medical balloons 1010. Further advantageously, in an example where a plurality of pumps are provided, the flow of inflation fluid to each inflatable medical balloon 1010, and inflatable medical balloon 1020, can be individually controlled.

In one example, where inflatable medical balloons 1010 are in contact with each other, sensor data unit 90 determines for each inflatable medical balloon 1010 that it is in contact with the wall of the patient lumen responsive to a predetermined function of the output of the respective pressure sensor and the outputs of the pressure sensors of the adjacent inflatable medical balloons 1010. In another example, where inflatable medical balloons 1010 are in contact with inflatable medical balloon 1020, sensor data unit 90 determines for each inflatable medical balloon 1010 that it is in contact with the wall of the patient lumen responsive to a predetermined function of the output of the respective pressure sensor and the output of the pressure sensor of inflatable medical balloons 1020.

In another example, for each inflatable medical balloon 1010, sensor data unit 90 determines an extent of calcification the portion of the patient lumen juxtaposed with the respective inflatable medical balloon 1010. Particularly, due to the fact that each inflatable medical balloon 1010 covers only a portion of the circumference of the patient lumen, as opposed to inflatable medical balloon 40 which covers the entire circumference of the patient lumen, sensor data unit 90 is able to more accurately determine an extent of calcification pressing against the respective inflatable medical balloon 1010 responsive to an output of a respective pressure sensor positioned within the respective inflation fluid flow. In one further example, sensor data unit 90 generates a map of the calcification of the patient lumen.

In another example, where respective sets of pressure sensors are juxtaposed with a surface of inflatable medical balloons 1010, for each inflatable medical balloon 1010 sensor data unit 90 determines a surface topography of a portion of the patient lumen juxtaposed with the respective inflatable medical balloon 1010 and/or determines an extent of calcification in the portion of the patient lumen juxtaposed with the respective inflatable medical balloon 1010, as described above. In one further example, sensor data unit 90 further generates a 3D map of the surface topography of the patient lumen, as described above. In another further example, sensor data unit 90 further generates a 4D map of the surface topography and the extent of calcification of the patient lumen, as described above.

In another example, where respective sets of flex sensors are juxtaposed with a surface of inflatable medical balloons 1010, for each inflatable medical balloon 1010 sensor data unit 90 determines a surface topography of a portion of the patient lumen juxtaposed with the respective inflatable medical balloon 1010, as described above. In one further example, sensor data unit 90 generates a 3D map of the surface topography of the patient lumen responsive to the determined surface topography of each portion. In another example, where respective sets of flex sensors are juxtaposed with a surface of inflatable medical balloons 1010, for each inflatable medical balloon 1010 sensor data unit 90 determines a diameter thereof, as described above. In one further example, sensor data unit 90 further determines a diameter of the patient lumen responsive to the determined diameters of inflatable medical balloons 1010. In another example, where respective sets of flex sensors are juxtaposed with a surface of inflatable medical balloons 1010, for each inflatable medical balloon 1010 sensor data unit 90 determines whether a hazardous calcification is juxtaposed therewith, as described above.

In another example, where one or more diameter sensors are secured to each inflatable medical balloon 1010, as described above in relation to diameter sensors 60 of medical balloons sensing assembly 10, sensor data unit 90 determines the diameter of each inflatable medical balloon 1010. In one example, as described above, the diameter is determined responsive to an indication of a respective pressure sensor that the respective inflatable medical balloon 1010 is in contact with the wall of the patient lumen.

In another example, where one or more ultrasound transducers are provided within each inflatable medical balloon 1010, sensor data unit 90 determines the diameter of each inflatable medical balloon 1010, an indication of the surface topography of the patient lumen and/or an indication of the density of the surface of the patient lumen, as described above. In one further example, sensor data unit 90 is further arranged to determine the diameter of the patient lumen responsive to the determined diameters of inflatable medical balloons 1010. In another further example, sensor data unit 90 is further arranged to determine the extent of calcification of the surface of the patient lumen responsive to density indication provided by each ultrasound transducer, as described above.

In another example, where one or more thermal sensors are juxtaposed with each inflatable medical balloon 1010, sensor data unit 90 determines, for each inflatable medical balloon 1010, an indication of the rate of thermal dispersion of the portion of the patient lumen juxtaposed with the respective inflatable medical balloon 1010. In one further example, sensor data unit 90 further generates a 3D map of the rate of thermal dispersion of the patient lumen, as described above. In another further example, sensor data unit 90 further generates a 4D map of the patient lumen, as described above.

In another example (not shown), a plurality of imaging markers are disposed on inflatable medical balloons 1010. In such an example, one or more imagers image the imaging markers and the surface topography of respective portions of the surface of the patient lumen are determined, as described above.

FIG. 11D illustrates a high-level perspective view of a medical balloon sensing assembly 1100 and FIG. 11E illustrates a high-level cut-away view of medical balloon sensing assembly 1100, in accordance with certain examples. Medical balloon sensing assembly 1100 is in all respects similar to medical balloon sensing assembly 1000, with the exception that an inflatable medical balloon 1020 is not provided.

FIGS. 1-11 have been provided herein where medical balloon sensing assemblies 10, 150, 300, 400, 500, 600, 650, 700, 800, 850, 950, 1000 and 1100 are described separately, however this is not meant to limit each of medical balloon sensing assemblies 10, 150, 300, 400, 500, 600, 650, 700, 800, 850, 950, 1000 and 1100 to be separate examples, and combinations of the features thereof are particularly contemplated. Particularly, any of the above medical balloon sensing assemblies can be provided with one or more sensor position members. Similarly, any of the above medical balloon sensing assemblies can be provided with: one or more force sensors, such as one or more pressure sensors; one or more flex sensors; one or more thermal sensors, including one or more thermal cameras; one or more optical sensors, including one or more optical cameras; one or more flow sensors; one or more ultrasound transducers; and/or one or more imaging markers. Additionally, any of the above medical balloon sensing assemblies can be provided with inflatable medical balloon 10, inflatable medical balloon 200, inflatable medical balloons 1010 and/or inflatable medical balloon 1020.

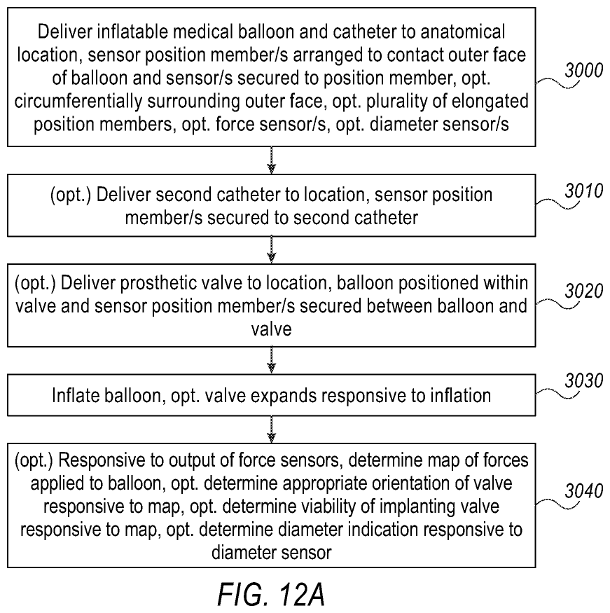

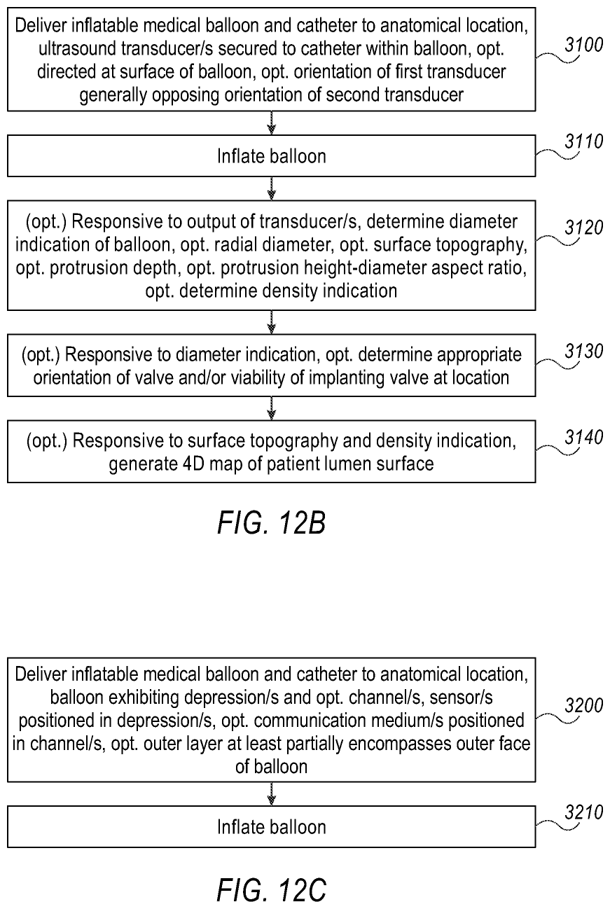

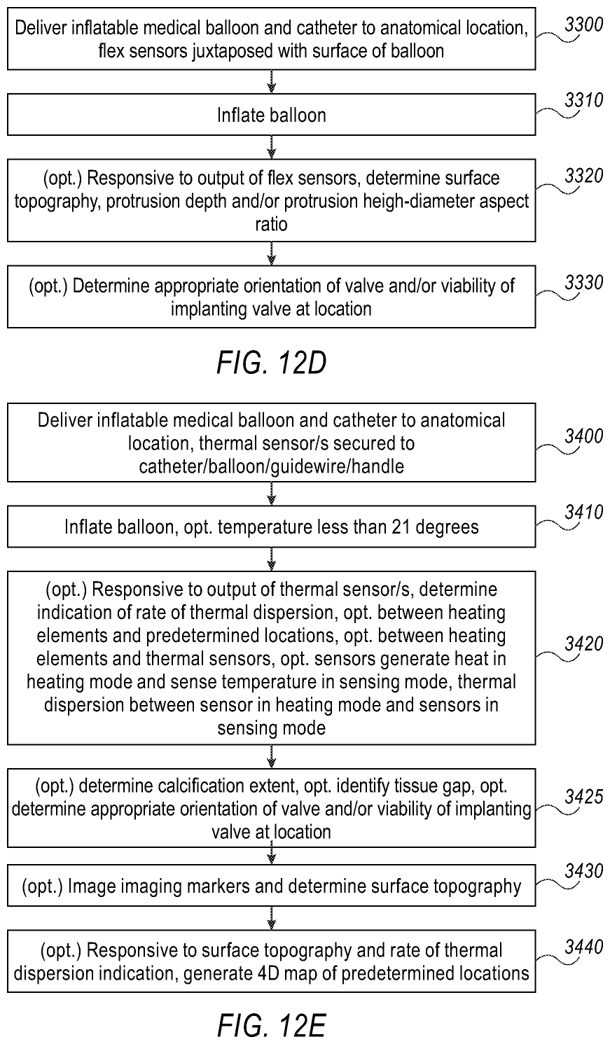

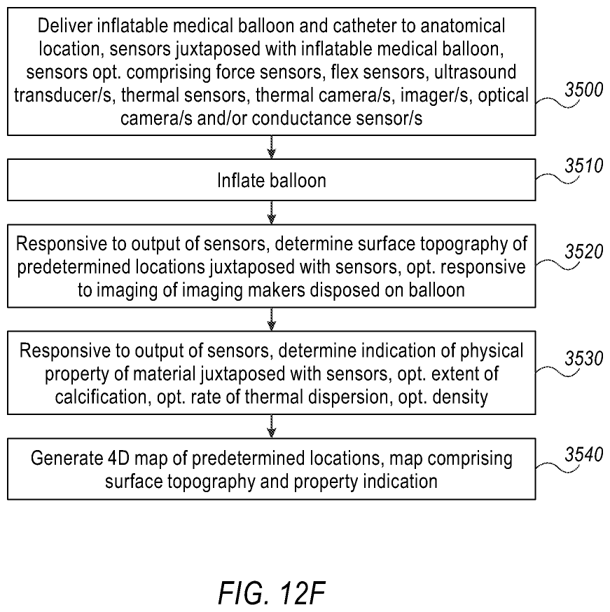

FIG. 12A illustrates a high-level flow chart of a first medical balloon sensing method, in accordance with certain examples. In stage 3000, an inflatable medical balloon and a first catheter are delivered to a predetermined anatomical location, such as the location of a defective heart valve, the inflatable medical balloon being secured to the first catheter. At least one sensor position member is arranged to contact an outer face of the inflatable medical balloon, and at least one sensor is secured to the at least one sensor position member. In one example, the at least one sensor is positioned on an outer face and/or inner face of the at least one sensor position member. In another example, the at least one sensor is embedded within the material of the at least one sensor position member. In one example, the at least one sensor position member at least partially circumferentially surrounds the outer face of the inflatable medical balloon. In another example, the at least one sensor position member comprises at least one elongated arm, each elongated arm having positioned thereon one or more sensors. In one further example, the at least one elongated arm comprises a plurality of elongated arms. In another further example, each elongated arm extends longitudinally along the outer face of the inflatable medical balloon.

In one example, the at least one sensor comprises one or more force sensors. Responsive to an output of each force sensor, the force and/or pressure applied thereto is measured. In one further example, the at least one force sensor comprises a plurality of force sensors. In another example, the at least one sensor comprises one or more diameter sensors.

In optional stage 3010, a second catheter is delivered to the predetermined anatomical location of stage 3000. In such an example, the at least one sensor position member of stage 3000 is secured to the second catheter. In optional stage 3020, a prosthetic valve is delivered to the predetermined anatomical location of stage 3000. In such an example, the inflatable medical balloon of stage 3000 is positioned within the prosthetic valve. In one example, the at least one sensor position member of stage 3000 is secured between the inflatable medical balloon and the prosthetic valve. In another example, stages 3000-3020 are performed simultaneously.

In stage 3030, the delivered inflatable medical balloon of stage 3000 is inflated. In one example, the delivered prosthetic valve of optional stage 3020 expands responsive to the inflation of the delivered inflatable medical balloon. In one further example, as the inflatable medical balloon inflates the expansion thereof pushes against the prosthetic valve to expand the prosthetic valve.

In optional stage 3040, responsive to an output of the plurality of force sensors of stage 3000, a map of forces applied to the inflatable medical balloon is determined. As described above, the map of forces includes a map of forces and/or pressure. In one example, an indication of the determined map of forces is output. In another example, responsive to the determined map of forces, an appropriate orientation for a prosthetic heart valve is determined, as described above, and an indication of the determined orientation is output. In another example, responsive to the determined map of forces, the viability of implanting a balloon expandable prosthetic valve at the anatomical location of stage 3000 is determined, as described above, and an indication of the determined viability is output. In another example, responsive to an output of the one or more diameter sensors of stage 3000, a diameter indication of the inflatable medical balloon is determined, as described above.

FIG. 12B illustrates a high-level flow chart of a second medical balloon sensing method, in accordance with certain examples. In stage 3100, an inflatable medical balloon and a catheter are delivered to a predetermined anatomical location, such as the location of a defective heart valve, the inflatable medical balloon being secured to the catheter. At least one ultrasound transducer is positioned on the catheter within the inflatable medical balloon, i.e. the at least one ultrasound transducer is positioned inside an enclosure formed by the inflatable medical balloon. In one example, the at least one ultrasound transducer is directed at a surface of the inflatable medical balloon. In another example, the at least one ultrasound transducer comprises at least two ultrasound transducers, an orientation of a first of the ultrasound transducers generally opposing an orientation of a second ultrasound transducer, as described above. In stage 3110, the delivered inflatable medical balloon of stage 3000 is inflated.

In optional stage 3120, responsive to an output of the at least one ultrasound transducer, a diameter indication of the inflatable medical balloon is determined and an indication of the determined diameter indication is output. In one example, the diameter indication comprises a radial diameter of the inflatable medical balloon. In another example, the diameter indication comprises a surface topography of the inflatable medical balloon and/or the patient lumen, as described above. In another example, the diameter indication comprises a protrusion depth of one or more protrusions extending into the inflatable medical balloon. In another example, the diameter indication comprises a height-diameter aspect ratio of one or more protrusions extending into the inflatable medical balloon. As described above, the protrusion depth and height-diameter aspect ratio indicate the severity of calcifications. In another example, responsive to an output of the at least one ultrasound transducer, an indication of the density of the surface of the patient lumen is determined.

In optional stage 3130, in one example, responsive to the determined diameter indication, an appropriate orientation for a prosthetic heart valve is determined, as described above, and an indication of the determined orientation is output. In another example, responsive to the determined diameter indication, the viability of implanting a balloon expandable prosthetic valve at the anatomical location of stage 3100 is determined, as described above, and an indication of the determined viability is output.

In optional stage 3140, in one example, responsive to the determined surface topography and density indication, a 4D map of the surface of the patient lumen is generated, the 4D map comprising: the 3D surface topography of the patient lumen; and the density indication, as described above.

FIG. 12C illustrates a high-level flow chart of a third medical balloon sensing method, in accordance with certain examples. In stage 3200, an inflatable medical balloon and a catheter are delivered to a predetermined anatomical location, such as the location of a defective heart valve, the inflatable medical balloon being secured to the catheter. The inflatable medical balloon exhibits at least one depression and at least one sensor is positioned within the at least one depression. In one example, the inflatable medical balloon further exhibits at least one channel and at least one communication medium is positioned within the at least one channel. In such an example, the at least one sensor is in communication with a sensor data unit via the at least one communication medium. In another example, an outer layer at least partially encompasses an outer face of the inflatable medical balloon. In such an example, the at least one sensor faces an inner face of the outer layer. In stage 3210, the inflatable medical balloon of stage 3200 is inflated.

FIG. 12D illustrates a high-level flow chart of a fourth medical balloon sensing method, in accordance with certain examples. In stage 3300, an inflatable medical balloon and a catheter are delivered to a predetermined anatomical location, such as the location of a defective heart valve, the inflatable medical balloon being secured to the catheter. A plurality of flex sensors are juxtaposed with a surface of the inflatable medical balloon. In one example, the flex sensors are positioned on one or more sensor position members, as described above. In another example, the flex sensors are positioned within one or more depressions of the inflatable medical balloon, as described above. In another example, the flex sensors are positioned on an outer face of the inflatable medical balloon. In another example, the flex sensors are positioned on an inner face of the inflatable medical balloon.

In stage 3310, the inflatable medical balloon is inflated. In optional stage 3320, responsive to an output of the plurality of the flex sensors of stage 3300, in one example, a surface topography of the inflatable medical balloon is determined and an indication of the determined surface topography is output. In another example, responsive to the output of the plurality of flex sensors, a protrusion depth of one or more protrusions extending into the inflatable medical balloon is determined and an indication of the determined protrusion depth is output. In another example, responsive to the output of the plurality of flex sensors, a height-diameter aspect ratio of one or more protrusions extending into the inflatable medical balloon is determined and an indication of the determined ratio is output.

In optional stage 3330, responsive to the determined surface topography, protrusion depth and/or height-diameter aspect ratio of optional stage 3320, an appropriate orientation for a prosthetic heart valve is determined, as described above, and an indication of the determined orientation is output. In another example, responsive to the determined surface topography, protrusion depth and/or height-diameter aspect ratio of optional stage 3320, the viability of implanting a balloon expandable prosthetic valve at the anatomical location of stage 3300 is determined, as described above, and an indication of the determined viability is output.

FIG. 12E illustrates a high-level flow chart of a fifth medical balloon sensing method, in accordance with certain examples. In stage 3400, an inflatable medical balloon and a catheter are delivered to a predetermined anatomical position, such as the location of a defective heart valve, the inflatable medical balloon being secured to the catheter. One or more thermal sensors are juxtaposed to the inflatable medical balloon and optionally secured to the inflatable medical balloon, the catheter, a guidewire and/or a handle. In one example, the one or more thermal sensors comprise one or more thermal cameras. In another example, the one or more thermal sensors comprise one or more thermistors. In one example, at least some of the thermal sensors are secured to, or embedded in, a surface of the inflatable medical balloon. In another example, at least some of the thermal sensors are secured to one or more sensor position members. In another example, one or more thermal cameras are secured to the catheter. In another example, one or more thermal cameras are secured to a guidewire. In another example, one or more thermal cameras are secured to a movable arm.

In stage 3410, the inflatable medical balloon is inflated. In one example, the inflatable medical balloon exhibits a temperature less than 21 degrees Celsius. In another example, the inflatable medical balloon is inflated with inflation fluid, the inflation fluid exhibiting a temperature less than 21 degrees Celsius.

In optional stage 3420, responsive to an output of the one or more thermal sensors of stage 3400, an indication of a rate of thermal dispersion of a plurality of predetermined locations juxtaposed with the inflatable medical balloon is determined and the indication is output. In one example, the thermal dispersion is between a plurality of heating elements and the predetermined locations. In another example, the thermal dispersion is between a plurality of heating elements and a plurality of thermal sensors. In another example, the thermal sensors are arranged to alternately operate in a heating mode and a sensing mode. In the heating mode the thermal sensors generate heat. In the sensing mode, the thermal sensors sense the temperature thereat, i.e. the temperature applied to each respective sensor. In such an example, the thermal dispersion is between a thermal sensor operating in the heating mode and associated thermal sensors operating in the sensing mode.

In optional stage 3425, responsive to the determined indication of optional stage 3420, the extent of calcification of the area of the patient lumen is determined and information regarding the extent of calcification is output. In another example, responsive to the determined indication, the locations and dimensions of calcifications are determined and output. In one example, responsive to the determined indication, a gap in tissue is identified. Optionally, an indication of the tissue gap is further output. In one further example, responsive to identified tissue gap, the inflatable medical balloon is inflated further.

In one example, responsive to the determined indication of the rate of thermal dispersion, an appropriate orientation for a prosthetic heart valve is determined, as described above, and an indication of the determined orientation is output. In another example, responsive to the determined indication of the rate of thermal dispersion, the viability of implanting a balloon expandable prosthetic valve at the anatomical location of stage 3400 is determined, as described above, and an indication of the determined viability is output.

In optional stage 3430, imaging markers positioned on the inflatable medical balloon of stage 3400 are imaged. In one example, the imaging markers are imaged by one or more optical cameras. In another example, the imaging markers are imaged by one or more x-ray based imagers, such as a fluoroscopic imager and/or a CT imager. In another example, the imaging markers are imaged by one or more infrared cameras. In another example, the imaging markers are imaged by one or more ultrasound transducers. Responsive to the imaging, a surface topography of the patient lumen is determined. In one further example, a 3D map of the surface of the patient lumen is generated and output.

In optional stage 3440, responsive to the determined surface topography of optional stage 3430 and the determined indication of the rate of thermal dispersion of optional stage 3420, a 4D map of the surface of the predetermined locations of optional stage 3420 is generated and output. Particularly, the 4D map comprises the determined surface topography and the indication of the rate of thermal dispersion.

FIG. 12F illustrates a high-level flow chart of a sixth medical balloon sensing method, in accordance with certain examples. In stage 3500, an inflatable medical balloon and a catheter are delivered to a predetermined anatomical position, such as the location of a defective heart valve, the inflatable medical balloon being secured to the catheter. One or more sensors are juxtaposed with the inflatable medical balloon. In one example, the one or more sensors comprise: a plurality of force sensors, optionally comprising pressure sensors; a plurality of flex sensors; one or more ultrasound transducers; a plurality of thermal sensors; one or more thermal cameras; one or more optical cameras; one or more imagers; and/or one or more conductance sensors.

In stage 3510, the inflatable medical balloon is inflated. In one example, the inflatable medical balloon exhibits a temperature less than 21 degrees Celsius. In another example, the inflatable medical balloon is inflated with inflation fluid, the inflation fluid exhibiting a temperature less than 21 degrees Celsius.

In stage 3520, responsive to an output of one or more of the one or more sensors of stage 3500, a surface topography of a plurality of predetermined locations juxtaposed with the one or more sensors is determined. As described above, in one example the plurality of predetermined locations form a predetermined area. In one example, a plurality of imaging markers are disposed on the inflatable medical balloon. In such an example, the imaging markers are imaged, the surface topography determined responsive to the imaging of the imaging markers.

In stage 3530, responsive to an output of one or more of the one or more sensors of stage 3500, an indication of a physical property of the material of the plurality of predetermined locations of stage 3520 is determined. In one example, the physical property comprises the rate of thermal dissipation therethrough. In another example, the physic property comprises the density thereof. In another example, the physical property comprises the extent of calcification thereat. In another example, the physical property comprises the electrical conductance thereof.

In stage 3540, a 4D map of the plurality of predetermined locations of stage 3520 is generated. The 4D map comprises the determined surface topography of stage 3520 and the physical property indication of stage 3530. The 4D map is then output.

Thus, various examples of medical balloon sensing assemblies are herein described. In one example, as described above in relation to FIGS. 1A-1L, one or more sensors are secured to at least one sensor position member, optionally constructed as a sleeve. In one further example, the at least one sensor position member is secured to a separate catheter. In another example, as described above in relation to FIG. 2, one or elongated sensor position members are provided, and one or more sensors are secured to the elongated sensor position members.

In one example, a map of forces and/or pressure is measured by the sensors. In another example, where the sensors comprise flex sensors, a surface topography of the medical balloon is determined. In one example, properties of various calcifications within the patient lumen are determined. In another example, the orientation of a prosthetic valve, and/or the viability of implanting a balloon expandable valve, is determined. In one example, a diameter indication of the medical balloon is determined. In another example, a predetermined function of the pressure applied to the medical balloon and the diameter of the medical balloon is determined. In one example, an indication of a rate of thermal dispersion is determined. In another example, a 4D map of the surface of the patient lumen is generated, the 4D map comprising: a 3D surface topography; and the indication of the rate of thermal dispersion.

In one example, as described above in relation to FIGS. 3A-3B, the one or more sensors are positioned within one or more depressions in the medical balloon. In another example, as described above in relation to FIGS. 4A-4C, the medical balloon is positioned within a prosthetic valve. In one further example, the at least one sensor position member is secured by the medical balloon and the prosthetic valve. In one example, as described above in relation to FIGS. 5A-5C, one or more ultrasound transducers are positioned on the catheter of the medical balloon. In one further example, a diameter indication of the medical balloon is determined responsive to the one or more ultrasound transducers. In another example, as described above in relation to FIG. 6, a controllable pump is provided, the controllable pump arranged to control the inflation of the medical balloon. In one further example, the controllable pump is responsive to the determined diameter indication and/or force/pressure measurements.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A medical balloon sensing assembly, comprising: a handle; a first catheter extending distally from the handle; an inflatable medical balloon secured by the first catheter; at least one sensor position member; and at least one sensor secured to the at least one sensor position member, wherein the at least one sensor position member is arranged to contact an outer face of the inflatable medical balloon.

Example 2. The medical balloon sensing assembly of any example herein, particularly example 1, wherein the at least one sensor position member at least partially circumferentially surrounds the outer face of the inflatable medical balloon.

Example 3. The medical balloon sensing assembly of any example herein, particularly any one of examples 1 or 2, wherein the at least one sensor position member comprises a sleeve, the sleeve covering the outer face of the inflatable medical balloon.

Example 4. The medical balloon sensing assembly of any example herein, particularly any one of examples 1 or 2, wherein the at least one sensor position member comprises at least one elongated arm.

Example 5. The medical balloon sensing assembly of any example herein, particularly example 4, wherein the at least one elongated arm comprises a plurality of elongated arms.

Example 6. The medical balloon sensing assembly of any example herein, particularly any one of examples 1 or 2, wherein the at least one sensor position member comprises a collapsible stent.

Example 7. The medical balloon sensing assembly of any example herein, particularly any one of examples 1 to 6, further comprising a second catheter extending distally from the handle, the at least one sensor position member secured to the second catheter.

Example 8. The medical balloon sensing assembly of any example herein, particularly any one of examples 1 to 7, further comprising a prosthetic valve, the inflatable medical balloon positioned within the prosthetic valve such that the prosthetic valve expands responsive to an inflation of the inflatable medical balloon.

Example 9. The medical balloon sensing assembly of any example herein, particularly example 8, wherein the at least one sensor position member is secured between the inflatable medical balloon and the prosthetic valve.

Example 10. The medical balloon sensing assembly of any example herein, particularly any one of examples 1 to 9, wherein the at least one sensor comprises at least one force sensor.

Example 11. The medical balloon sensing assembly of any example herein, particularly example 10, further comprising a sensor data unit in communication with the at least one force sensor, wherein the at least one force sensor comprises a plurality of force sensors, wherein the sensor data unit is arranged, responsive to an output of the plurality of force sensors, to: generate a map of forces applied to the inflatable medical balloon; and output an indication of the determined map.

Example 12. The medical balloon sensing assembly of any example herein, particularly any one of examples 1 to 7, further comprising a sensor data unit in communication with the at least one sensor, wherein the at least one sensor comprises a plurality of force sensors, and wherein the sensor data unit is arranged, responsive to an output of the plurality of force sensors, to: generate a map of forces applied to the inflatable medical balloon; responsive to the determined map of forces, determine an appropriate orientation for a prosthetic heart valve; and output an indication of the determined orientation.

Example 13. The medical balloon sensing assembly of any example herein, particularly any one of examples 1 to 7, further comprising a sensor data unit in communication with the at least one sensor, wherein the at least one sensor comprises a plurality of force sensors, and wherein the sensor data unit is arranged, responsive to an output of the plurality of force sensors, to: generate a map of forces applied to the inflatable medical balloon, responsive to the determined map of forces, determine whether implantation of a balloon expandable prosthetic valve at an anatomical location of the inflatable medical balloon is viable; and output an indication of the determined viability.

Example 14. The medical balloon sensing assembly of any example herein, particularly any one of examples 1 to 10, wherein the at least one sensor comprises a plurality of flex sensors.

Example 15. The medical balloon sensing assembly of any example herein, particularly example 14, further comprising a sensor data unit in communication with the plurality of flex sensors, wherein the sensor data unit is arranged, responsive to an output of the plurality of flex sensors, to: determine a surface topography of the inflatable medical balloon; and output an indication of the determined surface topography.

Example 16. The medical balloon sensing assembly of any example herein, particularly any one of examples 1 to 10 and 14, wherein the at least one sensor comprises: a diameter sensor, an output of the diameter sensor responsive to a radial diameter of the inflatable medical balloon; and a sensor data unit in communication with the diameter sensor, and wherein, responsive to the output of the diameter sensor, the control circuitry is arranged to: determine a diameter indication of the inflatable medical balloon; and output the determined diameter indication.

Example 17. The medical balloon sensing assembly of any example herein, particularly example 16, wherein the determined diameter indication comprises a change in a radial diameter of the inflatable medical balloon.

Example 18. The medical balloon sensing assembly of any example herein, particularly any one of examples 16 or 17, wherein the diameter sensor comprises: at least one radially translatable member juxtaposed with a surface of the inflatable medical balloon such that the inflation of the inflatable medical balloon radially translates the at least one radially translatable member; and a linear displacement sensor coupled to the at least one radially translatable member and in communication with the sensor data unit, an output of the linear displacement sensor configured to be responsive to the radial translation of the at least one radially translatable member.

Example 19. The medical balloon sensing assembly of any example herein, particularly example 18, wherein the at least one radially translatable member is circumferentially positioned on the outer face of the inflatable medical balloon.

Example 20. The medical balloon sensing assembly of any example herein, particularly any one of examples 16 or 17, wherein the diameter sensor comprises a strain gauge circumferentially position on the outer face of the inflatable medical balloon.

Example 21. The medical balloon sensing assembly of any example herein, particularly any one of examples 16 to 20, wherein the diameter indication comprises an indication of the recoil of the inflatable medical balloon.

Example 22. The medical balloon sensing assembly of any example herein, particularly any one of examples 1 to 20, further comprising at least one ultrasound transducer positioned on the catheter within the inflatable medical balloon.

Example 23. The medical balloon sensing assembly of any example herein, particularly any one of examples 22, wherein the at least one ultrasound transducer is directed at a surface of the inflatable medical balloon.

Example 24. The medical balloon sensing assembly of any example herein, particularly any one of examples 22 or 23, wherein the at least one ultrasound transducer comprises at least two ultrasound transducers, an orientation of a first of the ultrasound transducers generally opposing an orientation of the second of the ultrasound transducers.

Example 25. The medical balloon sensing assembly of any example herein, particularly any one of examples 1 to 24, further comprising: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein, responsive to the output of the at least one force sensor, the pump is arranged to adjust the flow of the inflation fluid.

Example 26. A medical balloon sensing assembly, comprising: a handle: a first catheter extending distally from the handle; an inflatable medical balloon secured by the first catheter; and at least one ultrasound transducer positioned on the first catheter within the inflatable medical balloon.

Example 27. The medical balloon sensing assembly of any example herein, particularly example 26, wherein the at least one ultrasound transducer is directed at a surface of the inflatable medical balloon.

Example 28. The medical balloon sensing assembly of any example herein, particularly any one of examples 26 or 27, wherein the at least one ultrasound transducer comprises at least two ultrasound transducers, an orientation of a first of the ultrasound transducers generally opposing an orientation of the second of the ultrasound transducers.

Example 29. The medical balloon sensing assembly of any example herein, particularly any one of examples 26 to 28, further comprising a sensor data unit in communication with the at least one ultrasound transducer, wherein the sensor data unit is arranged, responsive to an output of the at least one ultrasound transducer, to: determine a diameter indication of the inflatable medical balloon; and output an indication of the determined diameter indication.

Example 30. The medical balloon sensing assembly of any example herein, particularly example 29, wherein the diameter indication comprises a radial diameter of the inflatable medical balloon.

Example 31. The medical balloon sensing assembly of any example herein, particularly example 29, wherein the diameter indication comprises a surface topography of the inflatable medical balloon.

Example 32. The medical balloon sensing assembly of any example herein, particularly example 29, wherein the diameter indication comprises a protrusion depth of one or more protrusions extending into the inflatable medical balloon.

Example 33. The medical balloon sensing assembly of any example herein, particularly example 29, wherein the diameter indication comprises a height-diameter aspect ratio of one or more protrusions extending into the inflatable medical balloon.

Example 34. The medical balloon sensing assembly of any example herein, particularly example 29, wherein the diameter indication comprises an indication of the recoil of the inflatable medical balloon.

Example 35. The medical balloon sensing assembly of any example herein, particularly any one of examples 29 to 34, wherein the sensor data unit is further arranged, responsive to the determined diameter indication, to: determine an appropriate orientation for a prosthetic heart valve; and output an indication of the determined orientation.

Example 36. The medical balloon sensing assembly of any example herein, particularly any one of examples 29 to 34, wherein the sensor data unit is further arranged, responsive to the determined diameter indication, to: determine whether implantation of a balloon prosthetic valve at an anatomical location of the inflatable medical balloon is viable; and output an indication of the determined viability.

Example 37. The medical balloon sensing assembly of any example herein, particularly any one of examples 26 to 36, further comprising a prosthetic valve, the inflatable medical balloon positioned within the prosthetic valve such that the prosthetic valve expands responsive to an inflation of the inflatable medical balloon.

Example 38. The medical balloon sensing assembly of any example herein, particularly any one of examples 26 to 37, further comprising: at least one sensor position member; and at least one sensor secured to the at least one sensor position member, wherein the at least one sensor position member is arranged to contact an outer face of the inflatable medical balloon.

Example 39. The medical balloon sensing assembly of any example herein, particularly example 26, further comprising: at least one sensor position member; a plurality of force sensors secured to the at least one sensor position member; and a sensor data unit in communication with the plurality of force sensors, wherein the at least one sensor position member is arranged to contact an outer face of the inflatable medical balloon, and wherein the sensor data unit is arranged, responsive to an output of the plurality of force sensors, to: generate a map of forces applied to the inflatable medical balloon; and output an indication of the determined map.

Example 40. The medical balloon sensing assembly of any example herein, particularly any one of examples 26 to 37, further comprising at least one sensor, wherein the inflatable medical balloon exhibits at least one depression, the at least one sensor positioned within the at least one depression.

Example 41. The medical balloon sensing assembly of any example herein, particularly example 26, further comprising: at least one force sensor juxtaposed with the inflatable medical balloon; and a sensor data unit in communication with the at least one sensor, wherein, responsive to the output of the at least one force sensor, the sensor data unit is arranged to: determine a force applied to the inflatable medical balloon; and output an indication of the determined force.

Example 42. A medical balloon sensing assembly, comprising: a handle; a catheter extending distally from the handle; an inflatable medical balloon exhibiting at least one depression, the inflatable medical balloon secured by the catheter; and at least one sensor positioned within the at least one depression.

Example 43. The medical balloon sensing assembly of any example herein, particularly example 42, further comprising: a sensor data unit; and at least one communication medium, the at least one sensor in communication with the sensor data unit via the at least one communication medium, wherein the inflatable medical balloon further exhibits at least one channel, the at least one communication medium positioned within the at least one channel.

Example 44. The medical balloon sensing assembly of any example herein, particularly example 42 or example 43, further comprising an outer layer encompassing an outer face of the inflatable medical balloon, the at least one sensor facing an inner face of the outer layer.

Example 45. The medical balloon sensing assembly of any example herein, particularly any one of examples 42 to 44, wherein the at least one sensor comprises at least one force sensor.

Example 46. The medical balloon sensing assembly of any example herein, particularly any one of examples 42 or 44, further comprising a sensor data unit in communication with the at least one sensor, wherein the at least one sensor comprises a plurality of force sensors, wherein the sensor data unit is arranged, responsive to an output of the plurality of force sensors, to: generate a map of forces applied to the inflatable medical balloon; and output an indication of the determined map.

Example 47. The medical balloon sensing assembly of any example herein, particularly example 46, wherein, responsive to the determined map of forces, the sensor data unit is further arranged to: determine an appropriate orientation for a prosthetic heart valve; and output an indication of the determined orientation.

Example 48. The medical balloon sensing assembly of any example herein, particularly example 46 or example 47, wherein, responsive to the determined map of forces, the sensor data unit is further arranged to: determine whether implantation of a balloon expandable prosthetic valve at an anatomical location of the inflatable medical balloon is viable; and output an indication of the determined viability.

Example 49. The medical balloon sensing assembly of any example herein, particularly any one of examples 42 to 44, wherein the at least one sensor comprises a plurality of flex sensors, each of the plurality of flex sensors juxtaposed with the outer face of the inflatable medical balloon at a respective predetermined location.

Example 50. The medical balloon sensing assembly of any example herein, particularly example 42 or example 44, further comprising a sensor data unit in communication with the at least one sensor, wherein the at least one sensor comprises a plurality of flex sensors, each of the plurality of flex sensors juxtaposed with the outer face of the inflatable medical balloon at a respective predetermined location, and wherein the sensor data unit is arranged, responsive to an output of the plurality of flex sensors, to: determine a surface topography of the inflatable medical balloon; and output an indication of the determined surface topography.

Example 51. The medical balloon sensing assembly of any example herein, particularly any one of examples 42 or 44, wherein the at least one sensor comprises: a diameter sensor, an output of the diameter sensor responsive to a radial diameter of the inflatable medical balloon; and a sensor data unit in communication with the diameter sensor, wherein, responsive to the output of the diameter sensor, the control circuitry is arranged to: determine a diameter indication of the inflatable medical balloon; and output the determined diameter indication.

Example 52. The medical balloon sensing assembly of any example herein, particularly example 51, wherein the determined diameter indication comprises a change in a radial diameter of the inflatable medical balloon.

Example 53. The medical balloon sensing assembly of any example herein, particularly any one of examples 51 or 52, wherein the diameter sensor comprises: at least one radially translatable member juxtaposed with an outer face of the inflatable medical balloon such that the inflation of the inflatable medical balloon radially translates the at least one radially translatable member; and a linear displacement sensor coupled to the at least one radially translatable member and in communication with the sensor data unit, an output of the linear displacement sensor configured to be responsive to the radial translation of the at least one radially translatable member.

Example 54. The medical balloon sensing assembly of any example herein, particularly example 53, wherein the at least one radially translatable member is circumferentially positioned on the outer face of the inflatable medical balloon.

Example 55. The medical balloon sensing assembly of any example herein, particularly any one of examples 51 or 52, wherein the diameter sensor comprises a strain gauge circumferentially position on the outer face of the inflatable medical balloon.

Example 56. The medical balloon sensing assembly of any example herein, particularly any one of examples 42 to 55, further comprising at least one ultrasound transducer positioned on the catheter within the inflatable medical balloon.

Example 57. The medical balloon sensing assembly of any example herein, particularly example 56, wherein the at least one ultrasound transducer is directed at a surface of the inflatable medical balloon.

Example 58. The medical balloon sensing assembly of any example herein, particularly any one of examples 56 or 57, wherein the at least one ultrasound transducer comprises at least two ultrasound transducers, an orientation of a first of the ultrasound transducers generally opposing an orientation of the second of the ultrasound transducers.

Example 59. The medical balloon sensing assembly of any example herein, particularly any one of examples 42 to 58, further comprising: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein, responsive to the output of the at least one force sensor, the pump is arranged to adjust the flow of the inflation fluid.

Example 60. A medical balloon sensing assembly comprising: a handle; a first catheter extending distally from the handle; an inflatable medical balloon, the inflatable medical balloon secured by the first catheter; and a plurality of flex sensors juxtaposed with a surface of the inflatable medical balloon.

Example 61. The medical balloon sensing assembly of any example herein, particularly example 60, further comprising a sensor data unit in communication with the plurality of flex sensors, the sensor data unit arranged, responsive to an output of the plurality of flex sensors, to: determine a surface topography of the inflatable medical balloon; and output an indication of the determined surface topography.

Example 62. The medical balloon sensing assembly of any example herein, particularly example 60, further comprising a sensor data unit in communication with the plurality of flex sensors, the sensor data unit arranged, responsive to an output of the plurality of flex sensors, to: determine a protrusion depth of one or more protrusions extending into the inflatable medical balloon; and output an indication of the determined protrusion depth.

Example 63. The medical balloon sensing assembly of any example herein, particularly example 60, further comprising a sensor data unit in communication with the plurality of flex sensors, the sensor data unit arranged, responsive to an output of the plurality of flex sensors, to: determine a height-diameter aspect ratio of one or more protrusions extending into the inflatable medical balloon; and output an indication of the determined ratio.

Example 64. The medical balloon sensing assembly of any example herein, particularly any one of examples 60 to 63, further comprising at least one sensor position member arranged to contact an outer face of the inflatable medical balloon, the plurality of flex sensors secured to the at least one sensor position member.

Example 65. The medical balloon sensing assembly of any example herein, particularly example 64, wherein the at least one sensor position member comprises at least one elongated arm.

Example 66. The medical balloon sensing assembly of any example herein, particularly any one of examples 64 or 65, further comprising a second catheter extending distally from the handle, the at least one sensor position member secured to the second catheter.

Example 67. The medical balloon sensing assembly of any example herein, particularly example 60, further comprising: a diameter sensor, an output of the diameter sensor responsive to a radial diameter of the inflatable medical balloon; and a sensor data unit in communication with the diameter sensor, wherein, responsive to the output of the diameter sensor, the control circuitry is arranged to: determine a diameter indication of the inflatable medical balloon; and output the determined diameter indication.

Example 68. The medical balloon sensing assembly of any example herein, particularly any one of examples 60 to 67, further comprising at least one ultrasound transducer positioned on the first catheter within the inflatable medical balloon.

Example 69. The medical balloon sensing assembly of any example herein, particularly example 68, wherein the at least one ultrasound transducer is directed at a surface of the inflatable medical balloon.

Example 70. The medical balloon sensing assembly of any example herein, particularly any one of examples 68 or 69, wherein the at least one ultrasound transducer comprises at least two ultrasound transducers, an orientation of a first of the ultrasound transducers generally opposing an orientation of the second of the ultrasound transducers.

Example 71. The medical balloon sensing assembly of any example herein, particularly any one of examples 60 to 70, further comprising: a reservoir containing a predetermined volume of inflation fluid; and a pump in fluid communication with the reservoir and the inflatable medical balloon, wherein the pump is arranged to generate flow of the inflation fluid into the inflatable medical balloon.

Example 72. A process for preparing an inflatable medical balloon, the process comprising: inserting a predetermined volume of a polymer composition into a cavity of a mold, the mold exhibiting at least one protrusion or at least one depression; applying a predetermined pressure, at a predetermined temperature, to the inserted polymer composition to conform to the mold, thereby forming the inflatable medical balloon with at least one depression; removing the inflatable medical balloon from the mold; and positioning at least one sensor in the at least one depression of the inflatable medical balloon.

Example 73. The process of any example herein, particularly example 72, wherein at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped.

Example 74. The process of any example herein, particularly example 72, wherein at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

Example 75. The process of any example herein, particularly any one of examples 72 to 74, wherein the polymer composition is shaped as a tube.

Example 76. The process of any example herein, particularly any one of examples 72 to 74, wherein the polymer composition is in a molten state, the inserting comprising injecting the molten polymer composition into the cavity of the mold.

Example 77. The process of any example herein, particularly any one of examples 72 to 76, further comprising forming an outer layer encompassing an outer face of the inflatable medical balloon.

Example 78. The process of any example herein, particularly example 77, wherein the at least one sensor faces the outer layer.

Example 79. A process for preparing an inflatable medical balloon, the process comprising: coating a mold with a polymer composition or emulsion, the mold exhibiting at least one protrusion or at least one depression; drying and/or curing the polymer coating, thereby forming the inflatable medical balloon with at least one depression; removing the inflatable medical balloon from the mold; and positioning at least one sensor in the at least one depression of the inflatable medical balloon.

Example 80. The process of any example herein, particularly example 79, wherein at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being channel shaped.

Example 81. The process of any example herein, particularly example 79, wherein at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

Example 82. The process of any example herein, particularly any one of examples 79 to 81, wherein the coating the mold comprises dipping the mold within the polymer composition or emulsion.

Example 83. The process of any example herein, particularly any one of examples 79 to 82, further comprising forming an outer layer encompassing an outer face of the inflatable medical balloon.

Example 84. The process of any example herein, particularly example 83, wherein the at least one sensor faces the outer layer.

Example 85. An inflatable medical balloon prepared by a process comprising: inserting a predetermined volume of a polymer composition into a cavity of a mold, the mold exhibiting at least one protrusion or at least one depression; applying a predetermined pressure, at a predetermined temperature, to the inserted polymer composition to conform to the mold, thereby forming the inflatable medical balloon with at least one depression; removing the inflatable medical balloon from the mold; and positioning at least one sensor in the at least one depression of the inflatable medical balloon.

Example 86. The inflatable medical balloon of any example herein, particularly example 85, wherein at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped.

Example 87. The inflatable medical balloon of any example herein, particularly example 85, wherein at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being channel shaped.

Example 88. The inflatable medical balloon of any example herein, particularly any one of examples 85 to 87, wherein the polymer composition is shaped as a tube.

Example 89. The inflatable medical balloon of any example herein, particularly any one of examples 85 to 87, wherein the polymer composition is in a molten state, the inserting comprising injecting the molten polymer composition into the cavity of the mold.

Example 90. The inflatable medical balloon of any example herein, particularly any one of examples 85 to 89, wherein the process further comprising forming an outer layer encompassing an outer face of the inflatable medical balloon.

Example 91. The inflatable medical balloon of any example herein, particularly example 90, wherein the at least one sensor faces the outer layer.

Example 92. An inflatable medical balloon prepared by a process comprising: coating a mold with a polymer composition or emulsion, the mold exhibiting at least one protrusion or at least one depression; drying and/or curing the polymer coating, thereby forming the inflatable medical balloon with at least one depression; removing the inflatable medical balloon from the mold; and positioning at least one sensor in the at least one depression of the inflatable medical balloon.

Example 93. The inflatable medical balloon of any example herein, particularly example 92, wherein at least one protrusion of the mold comprises at least one first protrusion and at least one second protrusion, the at least one first protrusion arranged to form at least one first depression in the inflatable medical balloon and the at least one second protrusion arranged to form at least one second depression in the inflatable medical balloon, wherein the at least one sensor is positioned within the at least one first depression, and wherein the process further comprises positioning at least one communication medium within the at least one second depression, the at least one second depression being groove shaped.

Example 94. The inflatable medical balloon of any example herein, particularly example 92, wherein at least one depression of the mold comprises at least one first depression and at least one second depression, the at least one first depression of the mold arranged to form at least one first depression in the inflatable medical balloon and the at least one second depression of the mold arranged to form at least one second depression in the inflatable medical balloon, wherein the at least one sensor is positioned within the at least one first depression of the inflatable medical balloon, and wherein the process further comprises positioning at least one communication medium within the at least one second depression of the inflatable medical balloon, the second depression of the inflatable medical balloon being groove shaped.

Example 95. The inflatable medical balloon of any example herein, particularly any one of examples 92 to 94, wherein the coating the mold comprises dipping the mold within the polymer composition or emulsion.

Example 96. The inflatable medical balloon of any example herein, particularly any one of examples 92 to 95, further comprising forming an outer layer encompassing an outer face of the inflatable medical balloon.

Example 97. The inflatable medical balloon of any example herein, particularly example 96, wherein the at least one sensor faces the outer layer.

Example 98. A medical balloon sensing method, the method comprising: delivering an inflatable medical balloon and a first catheter to a predetermined anatomical location, the inflatable medical balloon secured to the first catheter, and inflating the delivered inflatable medical balloon, wherein at least one sensor position member is arranged to contact an outer face of the inflatable medical balloon, and wherein at least one sensor is secured to the at least one sensor position member.

Example 99. The method of any example herein, particularly example 98, wherein the at least one sensor position member at least partially circumferentially surrounds the outer face of the inflatable medical balloon.

Example 100. The method of any example herein, particularly example 98, wherein the at least one sensor position member comprises a plurality of elongated arms.

Example 101. The method of any example herein, particularly example 98, further comprising delivering a second catheter to the predetermined anatomical location, the at least one sensor position member secured to the second catheter.

Example 102. The method of any example herein, particularly any one of examples 98 to 101, further comprising delivering a prosthetic valve to the predetermined anatomical location, the inflatable medical balloon positioned within the prosthetic valve such that the prosthetic valve expands responsive to the inflation of the inflatable medical balloon.

Example 103. The method of any example herein, particularly example 102, wherein the at least one sensor position member is secured between the inflatable medical balloon and the prosthetic valve.

Example 104. The method of any example herein, particularly any one of examples 98 to 103, wherein the at least one sensor comprises a plurality of force sensors, the method further comprising, responsive to an output of the plurality of force sensors: determining a map of forces applied to the inflatable medical balloon; and outputting an indication of the determined map.

Example 105. The method of any example herein, particularly any one of examples 98 to 103, wherein the at least one sensor comprises a plurality of force sensors, the method further comprising, responsive to an output the plurality of force sensors: determining a map of forces applied to the inflatable medical balloon; responsive to the determined map of forces, determining an appropriate orientation for a prosthetic heart valve; and outputting an indication of the determined orientation.

Example 106. The method of any example herein, particularly any one of examples 95 to 100, wherein the at least one sensor comprises a plurality of force sensors, the method further comprising, responsive to an output the plurality of force sensors: determining a map of forces applied to the inflatable medical balloon; responsive to the determined map of forces, determining a viability of implanting a balloon expandable prosthetic valve at the predetermined anatomical location; and outputting an indication of the determined viability.

Example 107. A medical balloon sensing method, the method comprising: delivering an inflatable medical balloon and a first catheter to a predetermined anatomical location, the inflatable medical balloon secured to the first catheter; and inflating the delivered inflatable medical balloon, wherein at least one ultrasound transducer is positioned on the first catheter within the inflatable medical balloon.

Example 108. The method of any example herein, particularly any one of examples 107, wherein the at least one ultrasound transducer is directed at a surface of the inflatable medical balloon.

Example 109. The method of any example herein, particularly any one of examples 107 or 108, wherein the at least one ultrasound transducer comprises two ultrasound transducers, an orientation of a first of the ultrasound transducers generally opposing an orientation of the second of the ultrasound transducers.

Example 110. The method of any example herein, particularly any one of examples 107 to 109, further comprising, responsive to an output of the at least one ultrasound transducer: determining a diameter indication of the inflatable medical balloon; and outputting an indication of the determined diameter indication.

Example 111. The method of any example herein, particularly example 110, wherein the diameter indication comprises a radial diameter of the inflatable medical balloon.

Example 112. The method of any example herein, particularly example 110, wherein the diameter indication comprises a surface topography of the inflatable medical balloon.

Example 113. The method of any example herein, particularly example 110, wherein the diameter indication comprises an indication of the recoil of the inflatable medical balloon.

Example 114. The method of any example herein, particularly example 111, wherein the diameter indication comprises a protrusion depth of one or more protrusions extending into the inflatable medical balloon.

Example 115. The method of any example herein, particularly example 111, wherein the diameter indication comprises a height-diameter aspect ratio of one or more protrusions extending into the inflatable medical balloon.

Example 116. The method of any example herein, particularly any one of examples 111 to 115, further comprising, responsive to the determined diameter indication: determining an appropriate orientation for a prosthetic heart valve; and outputting an indication of the determined orientation.

Example 117. The method of any example herein, particularly any one of examples 111 to 116, further comprising, responsive to the determined diameter indication: determining whether implantation of a balloon prosthetic valve at the predetermined anatomical location is viable; and outputting an indication of the determined viability.

Example 118. A medical balloon sensing method, the method comprising: delivering an inflatable medical balloon and a first catheter to a predetermined anatomical location, the inflatable medical balloon secured to the first catheter; and inflating the delivered inflatable medical balloon, wherein the inflatable medical balloon exhibits at least one depression, at least one sensor position within the at least one depression.

Example 119. The method of any example herein, particularly example 118, wherein the inflatable medical balloon further exhibits at least one channel, at least one communication medium positioned within the at least one channel, the at least one sensor in communication with a sensor data unit via the at least one communication medium.

Example 120. The method of any example herein, particularly any one of examples 118 or 119, wherein an outer layer at least partially encompasses an outer face of the inflatable medical balloon, the at least one sensor facing an inner face of the outer layer.

Example 121. A medical balloon sensing method, the method comprising: delivering the inflatable medical balloon and a first catheter to a predetermined anatomical location, the inflatable medical balloon secured to the first catheter; and inflating the delivered inflatable medical balloon, wherein a plurality of flex sensors are juxtaposed with a surface of the inflatable medical balloon.

Example 122. The method of any example herein, particularly any one of examples 121, further comprising, responsive to an output of the plurality of flex sensors: determining a surface topography of the inflatable medical balloon; and outputting an indication of the determined surface topography.

Example 123. The method of any example herein, particularly any one of examples 121, further comprising, responsive to an output of the plurality of flex sensors: determining a protrusion depth of one or more protrusions extending into the inflatable medical balloon; and outputting an indication of the determined protrusion depth.

Example 124. The method of any example herein, particularly example 121, further comprising, responsive to an output of the plurality of flex sensors: determining a height-diameter aspect ratio of one or more protrusions extending into the inflatable medical balloon, and outputting an indication of the determined ratio.

Example 125. A medical balloon sensing assembly comprising: a handle; a catheter extending distally from the handle; an inflatable medical balloon secured by the catheter; at least one thermal sensor juxtaposed with the inflatable medical balloon; and a sensor data unit in communication with the at least one thermal sensor, wherein the sensor data unit is arranged, responsive to an output of the at least one thermal sensor, to: determine an indication of a rate of thermal dispersion between the inflatable medical balloon and a plurality of predetermined locations juxtaposed with the inflatable medical balloon; and output the determined indication of the rate of thermal dispersion.

Example 126. The medical balloon sensing assembly of any example herein, particularly example 125, wherein the plurality of predetermined locations constitutes a predetermined area surrounding the inflatable medical balloon.

Example 127. The medical balloon sensing assembly of any example herein, particularly example 125 or example 126, wherein the sensor data unit is further arranged, responsive to the determined indication of the rate of thermal dispersion, to: generate a map of the rate of thermal dispersion between the inflatable medical balloon and the plurality of predetermined locations; and output an indication of the determined map.

Example 128. The medical balloon sensing assembly of any example herein, particularly any one of examples 125 to 127, wherein the sensor data unit is further arranged, responsive to the determined indication of the rate of thermal dispersion, to: identify a tissue gap at one of the plurality of predetermined locations; and output an indication of the identified tissue gap.

Example 129. The medical balloon sensing assembly of any example herein, particularly example 128, further comprising: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein the sensor data unit is further arranged, responsive to the identification of a tissue gap, to control the pump to increase an amount of the inflation fluid pumped into the inflatable medical balloon.

Example 130. The medical balloon sensing assembly of any example herein, particularly any one of examples 125 to 129, wherein the sensor data unit is further arranged, for each of the plurality of predetermined locations and responsive to the respective determined indication of the rate of thermal dispersion, to: determine an extent of calcification at the respective predetermined location; and output an indication of the determined extent of calcification.

Example 131. The medical balloon sensing assembly of any example herein, particularly any one of examples 125 to 130, wherein the sensor data unit is further arranged, responsive to the determined indications of the rate of thermal dispersion, to: determine an appropriate orientation for a prosthetic heart valve; and output an indication of the determined orientation.

Example 132. The medical balloon sensing assembly of any example herein, particularly any one of examples 125 to 131, wherein the sensor data unit is further arranged, responsive to the determined indications of the rate of thermal dispersion, to: determine whether implantation of a balloon expandable prosthetic valve at an anatomical location of the inflatable medical balloon is viable; and output an indication of the determined viability.

Example 133. The medical balloon sensing assembly of any example herein, particularly any one of examples 125 to 132, further comprising a plurality of heating elements, each of the plurality of heating elements secured in relation to a respective predetermined point on a surface of the inflatable medical balloon, wherein the sensor data unit is further arranged to control the plurality of heating elements to generate heat at a predetermined temperature, the determined indication of the rate of thermal dispersion being between the plurality of heating elements and the plurality of predetermined locations.

Example 134. The medical balloon sensing assembly of any example herein, particularly example 133, wherein the at least one thermal sensor comprises a plurality of thermal sensors, each of the plurality of thermal sensors associated with a respective one of the plurality of heating elements, and wherein, for each of the plurality predetermined locations, the determination of the indication of the rate of thermal dispersion is responsive to a temperature difference between a respective one of the plurality of thermal sensors and the associated heating element.

Example 135. The medical balloon sensing assembly of any example herein, particularly any one of examples 125 to 133, wherein the at least one thermal sensor comprises a plurality of thermal sensors, wherein, responsive to the sensor data unit, each of the plurality of thermal sensors is arranged to alternately operate in a heating mode and a sensing mode, wherein in the heating mode the respective thermal sensor is arranged to generate heat at a predetermined temperature, and in the sensing mode the respective thermal sensor is arranged to sense a temperature thereat, wherein each of the plurality of thermal sensors is associated with another or others of the plurality of thermal sensors, and wherein the sensor data unit is further arranged, at each of a plurality of predetermined time points, to: control a respective one of the plurality of thermal sensors to operate in the heating mode; and control the thermal sensors associated with the respective thermal sensor to operate in the sensing mode, wherein the determination of the indication of the rate of thermal dispersion is responsive to a temperature difference between the respective thermal sensor operating in the heating mode and the associated thermal sensors operating in the sensing mode.

Example 136. The medical balloon sensing assembly of any example herein, particularly any one of examples 125 to 135, further comprising: a plurality of imaging markers; and an imager in communication with the sensor data unit, wherein the imager is arranged to image the plurality of imaging markers, and wherein the sensor data unit is further arranged, responsive to the output of the imager to: determine a surface topography of the plurality of predetermined locations; and generate a 4-dimensional (4D) map of the plurality of predetermined locations, the 4D map comprising the determined surface topography and the respective indications of the rate of thermal dispersion.

137. The medical balloon sensing assembly of any example herein, particularly any one of examples 125 to 135, further comprising: a plurality of imaging markers juxtaposed with a surface of the inflatable medical balloon; and an optical camera in communication with the sensor data unit, wherein the optical camera is arranged to image the plurality of imaging markers, and wherein the sensor data unit is further arranged, responsive to the output of the optical camera to: determine a surface topography of the plurality of predetermined locations; and generate a 4-dimensional (4D) map of the plurality of predetermined locations, the 4D map comprising the determined surface topography and the respective indications of the rate of thermal dispersion.

Example 138. The medical balloon sensing assembly of any example herein, particularly any one of examples 125 to 135, further comprising: a plurality of imaging markers; and a thermal camera in communication with the sensor data unit, wherein the thermal camera is arranged to image the plurality of imaging markers, and wherein the sensor data unit is further arranged, responsive to the output of the thermal camera to: determine a surface topography of the plurality of predetermined locations; and generate a 4-dimensional (4D) map of the plurality of predetermined locations, the 4D map comprising the determined surface topography and the respective indications of the rate of thermal dispersion.

Example 139. The medical balloon sensing assembly of any example herein, particularly any one of examples 125 to 138, wherein a temperature of an interior of the inflatable medical balloon is less than 21 degrees Celsius.

Example 140. The medical balloon sensing assembly of any example herein, particularly example 125, further comprising: a reservoir containing a predetermined volume of inflation fluid, the inflation fluid exhibiting a temperature of less than 21 degrees Celsius, wherein the reservoir is in fluid communication with the inflatable medical balloon.

Example 141. The medical balloon sensing assembly of any example herein, particularly example 140, further comprising a pump in fluid communication with the reservoir and the inflatable medical balloon, wherein the pump is arranged to generate flow of the inflation fluid into the inflatable medical balloon.

Example 142. A medical balloon sensing method, the method comprising: delivering an inflatable medical balloon and a catheter to a predetermined anatomical position, the inflatable medical balloon secured to the catheter; inflating the delivered inflatable medical balloon; determining an indication of a rate of thermal dispersion between the inflatable medical balloon and a plurality of predetermined locations juxtaposed with the inflatable medical balloon; and outputting the determined indication of the rate of thermal dispersion.

Example 143. The method of any example herein, particularly example 142, wherein the plurality of predetermined locations constitutes a predetermined area surrounding the inflatable medical balloon.

Example 144. The method of any example herein, particularly example 142 or example 143, further comprising: responsive to the determined indication of the rate of thermal dispersion, generating a map of the rate of thermal dispersion between the inflatable medical balloon and the plurality of predetermined locations; and outputting an indication of the determined map.

Example 145. The method of any example herein, particularly any one of examples 142 to 144, further comprising responsive to the determined indication of the rate of thermal dispersion, identifying a tissue gap at one of the plurality of predetermined locations.

Example 146. The method of any example herein, particularly example 145, further comprising: generating flow of an inflation fluid into the inflatable medical balloon; and responsive to the identification of a tissue gap, increasing an amount of the inflation fluid pumped into the inflatable medical balloon.

Example 147. The method of any example herein, particularly any one of examples 142 to 146, further comprising, for each of the plurality of predetermined locations and responsive to the respective determined indication of the rate of thermal dispersion: determining an extent of calcification at the respective predetermined location; and outputting an indication of the determined extent of calcification.

Example 148. The method of any example herein, particularly any one of examples 142 to 144, further comprising, responsive to the determined indications of the rate of thermal dispersion: determining an appropriate orientation for a prosthetic heart valve; and outputting an indication of the determined orientation.

Example 149. The method of any example herein, particularly any one of examples 142 to 148, further comprising, responsive to the determined indications of the rate of thermal dispersion: determining whether implantation of a balloon expandable prosthetic valve at an anatomical location of the inflatable medical balloon is viable; and outputting an indication of the determined viability.

Example 150. The method of any example herein, particularly any one of examples 142 to 149, further comprising controlling a plurality of heating elements to generate heat at a predetermined temperature, the plurality of heating elements secured in relation to a respective predetermined point on a surface of the inflatable medical balloon, wherein the determined indication of the rate of thermal dispersion being between the plurality of heating elements and the plurality of predetermined locations.

Example 151. The method of any example herein, particularly example 150, wherein, for each of the plurality predetermined locations, the determination of the indication of the rate of thermal dispersion is responsive to a temperature difference between a respective one of a plurality of thermal sensors and an associated one of the plurality of heating elements.

Example 152. The method of any example herein, particularly any one of examples 142 to 150, further comprising, at each of a plurality of predetermined time points: controlling a respective one of a plurality of thermal sensors to operate in a heating mode wherein the respective thermal sensor is arranged to generate heat at a predetermined temperature, each of the plurality of thermal sensors associated with another or others of the plurality of thermal sensors; and controlling the thermal sensors associated with the respective thermal sensor to operate in a sensing mode wherein the associated thermal sensors are each arranged to sense a temperature thereat, wherein the determination of the indication of the rate of thermal dispersion is responsive to a temperature difference between the respective thermal sensor operating in the heating mode and the associated thermal sensors operating in the sensing mode.

Example 153. The method of any example herein, particularly any one of examples 142 to 152, further comprising: imaging a plurality of imaging markers juxtaposed with a surface of the inflatable medical balloon; responsive to the imaging, determining a surface topography of the plurality of predetermined locations; and generating a 4-dimensional (4D) map of the plurality of predetermined locations, the 4D map comprising the determined surface topography and the respective indications of the rate of thermal dispersion.

Example 154. The method of any example herein, particularly any one of examples 142 to 153, wherein a temperature of an interior of the inflatable medical balloon is less than 21 degrees Celsius.

Example 155. The method of any example herein, particularly example 142, further comprising generating flow of an inflation fluid into the inflatable medical balloon, the inflation fluid exhibiting a temperature of less than 21 degrees Celsius.

Example 156. A medical balloon sensing assembly, comprising: a handle; a plurality of catheters extending distally from the handle; and a plurality of first inflatable medical balloons, each of the first inflatable medical balloons secured by a respective one of the plurality catheters, wherein the plurality of first inflatable medical balloons are arranged in a radially arrayed configuration.

Example 157. The medical balloon sensing assembly of any example herein, particularly example 156, wherein each of the plurality of first inflatable medical balloons comprises a respective longitudinal axis, the longitudinal axes of the plurality of first inflatable medical balloons being generally in parallel with each other.

Example 158. The medical balloon sensing assembly of any example herein, particularly any one of examples 156 or 157, wherein each first inflatable medical balloon is arranged to contact each of a pair of first inflatable medical balloons adjacent thereto.

Example 159. The medical balloon sensing assembly of any example herein, particularly example 156, further comprising a second inflatable medical balloon secured by a respective one of the plurality of catheters, wherein the plurality of first inflatable medical balloons are radially arrayed about the second inflatable medical balloon.

Example 160. The medical balloon sensing assembly of any example herein, particularly example 159, wherein each of the plurality of first inflatable medical balloons comprises a respective longitudinal axis and the second inflatable medical balloon comprises a respective longitudinal axis, and wherein the longitudinal axes of the first inflatable medical balloons are generally in parallel with the longitudinal axis of the second inflatable medical balloon.

Example 161. The medical balloon sensing assembly of any example herein, particularly any one of examples 159 or 160, wherein each of the plurality of first inflatable medical balloons is arranged to contact the second inflatable medical balloon.

Example 162. The medical balloon sensing assembly of any example herein, particularly any one of examples 159 to 161, wherein each first inflatable medical balloon is arranged to contact each of a pair of first inflatable medical balloons adjacent thereto.

Example 163. The medical balloon sensing assembly of any example herein, particularly any one of examples 156 to 162, further comprising a plurality of pressure sensors, each of the plurality of pressure sensors juxtaposed with a respective input port of a respective one of the plurality of catheters.

Example 164. The medical balloon sensing assembly of any example herein, particularly any one of examples 156 to 163, further comprising a plurality of flow sensors, each of the plurality of flow sensors juxtaposed with a respective input port of a respective one of the plurality of catheters.

Example 165. The medical balloon sensing assembly of any example herein, particularly any one of examples 156 to 164, further comprising: a reservoir containing a predetermined volume of inflation fluid; and a plurality of pumps in fluid communication with the reservoir and the plurality of catheters, wherein each of the plurality of pumps is arranged to generate flow of the inflation fluid into a respective one of the plurality of first inflatable medical balloons via the respective one of the plurality of catheters.

Example 166. The medical balloon sensing assembly of any example herein, particularly example 165, further comprising a plurality of sensors, each of the plurality of sensors juxtaposed with a respective one of the plurality of first inflatable medical balloons or with a respective one of the plurality of catheters, wherein, responsive to the output of each of the plurality of sensors, the respective one of the plurality of pumps is arranged to adjust the flow of the inflation fluid in relation to the respective one of the plurality of first inflatable medical balloons.

Example 167. A medical balloon sensing assembly comprising: a handle; a catheter extending distally from the handle; an inflatable medical balloon secured by the catheter; one or more sensors juxtaposed with the inflatable medical balloon; and a sensor data unit in communication with the one or more sensors, wherein, responsive to an output of each of the one or more sensors, the sensor data unit is arranged to: determine a surface topography of a plurality of predetermined locations juxtaposed with the one or more sensors; determine an indication of a physical property of material of the plurality of predetermined locations; generate a 4-dimensional (4D) map of the plurality of predetermined locations, the 4D map comprising the determined surface topography and the determined indication of the physical property of the material; and output the generated 4D map.

Example 168. The medical balloon sensing assembly of any example herein, particularly example 167, further comprising a plurality of imaging markers disposed on the inflatable medical balloon, the one or more sensors comprising an imager arranged to image the imaging markers, wherein the determination of the surface topography is responsive to an output of the imager.

Example 169. The medical balloon sensing assembly of any example herein, particularly 167, further comprising a plurality of imaging markers disposed on the inflatable medical balloon, the one or more sensors comprising an optical camera arranged to image the imaging markers, wherein the determination of the surface topography is responsive to an output of the optical camera.

Example 170. The medical balloon sensing assembly of any example herein, particularly example 167, further comprising a plurality of imaging markers disposed on the inflatable medical balloon, the one or more sensors comprising an ultrasound transducer arranged to image the imaging markers, wherein the determination of the surface topography is responsive to an output of the ultrasound transducer.

Example 171. The medical balloon sensing assembly of any example herein, particularly example 167, further comprising a plurality of imaging markers disposed on the inflatable medical balloon, the one or more sensors comprising a thermal camera arranged to image the imaging markers, wherein the determination of the surface topography is responsive to an output of the thermal camera.

Example 172. The medical balloon sensing assembly of any example herein, particularly example 171, wherein the indication of a physical property comprises an indication of a rate of thermal dispersion, the indication of the rate of thermal dispersion determined responsive to an output of the thermal camera.

Example 173. The medical balloon sensing assembly of any example herein, particularly any one of examples 167 to 171, wherein the indication of a physical property comprises an indication of a rate of thermal dispersion, and wherein the plurality of sensors comprises a plurality of thermal sensors, the indication of the rate of thermal dispersion determined responsive to respective outputs of the plurality of thermal sensors.

Example 174. The medical balloon sensing assembly of any example herein, particularly any one of examples 167 to 169 and 172, wherein the indication of a physical property comprises a density, and wherein the plurality of sensors comprises an ultrasound transducer, the indication of the density determined responsive to an output of the ultrasound transducer.

Example 175. The medical balloon sensing assembly of any example herein, particularly example 167, further comprising a plurality of imaging markers disposed on the inflatable medical balloon, the one or more sensors comprising an ultrasound transducer arranged to image the imaging markers, wherein the determination of the surface topography is responsive to an output of the ultrasound transducer, and wherein the indication of a physical property comprises a density the indication of the density determined responsive to an output of the ultrasound transducer.

Example 176. The medical balloon sensing assembly of any example herein, particularly any one of examples 167 to 171, wherein the indication of a physical property comprises an extent of calcification.

Example 177. An expandable structure sensing method, the method comprising: delivering an expandable structure and a catheter to a predetermined anatomical location, the expandable structure secured to the catheter; inflating the delivered expandable structure; determining a surface topography of a plurality of predetermined locations juxtaposed with the expandable structure; determining an indication of a physical property of material of the plurality of predetermined locations; generating a 4-dimensional (4D) map of the plurality of predetermined locations, the 4D map comprising the determined surface topography and the determined indication of the physical property of the material; and outputting the generated 4D map.

Example 178. The method of any example herein, particularly example 177, further comprising imaging a plurality of imaging markers disposed on the expandable structure, wherein the determination of the surface topography is responsive to the imaging.

Example 179. The method of any example herein, particularly any one of examples 177 or 178, wherein the indication of a physical property comprises an indication of a rate of thermal dispersion.

Example 180. The method of any example herein, particularly any one of examples 177 or 178, wherein the indication of a physical property comprises a density.

Example 181. The method of any example herein, particularly any one of examples 177 or 178, wherein the indication of a physical property comprises an extent of calcification.

Although the above examples are described in relation to an inflatable medical balloon, this is not meant to be limiting in any way. In another example, the disclosed features may be used in coordination with any expandable structure that is expanded via inflation or another expansion mechanism without departing from the scope of the disclosure. Thus, for any of the examples disclosed herein, the inflatable medical balloon can be replaced with an expandable structure that can be expanded without the use of an inflation fluid. One example of an expandable structure can comprise a braided or woven structure, which can be self-expandable or expandable via one or more mechanical actuators. A self-expandable structure can be braided or woven from wires or filaments made of a shape memory material, such as Nitinol. In some examples, the expandable structure (such as a braided structure) can also be configured to receive an inflation fluid to facilitate expansion of the structure.

It is appreciated that certain features of the invention, which are, for clarity, described in the context of separate examples, may also be provided in combination in a single example. Conversely, various features of the invention which are, for brevity, described in the context of a single example, may also be provided separately or in any suitable subcombination. In particular, the invention has been described with an identification of each powered device by a class, however this is not meant to be limiting in any way. In an alternative example, all powered device are treated equally, and thus the identification of class with its associated power requirements is not required.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention includes both combinations and subcombinations of the various features described hereinabove as well as variations and modifications thereof which would occur to persons skilled in the art upon reading the foregoing description.

1. A medical balloon sensing assembly, comprising: a handle; a first catheter extending distally from the handle; an inflatable medical balloon secured by the first catheter; and at least one ultrasound transducer positioned on the first catheter within the inflatable medical balloon.

2. The medical balloon sensing assembly of claim 1, wherein the at least one ultrasound transducer is directed at a surface of the inflatable medical balloon.

3. The medical balloon sensing assembly of claim 1, wherein the at least one ultrasound transducer comprises at least two ultrasound transducers, an orientation of a first of the ultrasound transducers generally opposing an orientation of the second of the ultrasound transducers.

4. The medical balloon sensing assembly of claim 1, further comprising a sensor data unit in communication with the at least one ultrasound transducer, wherein the sensor data unit is arranged, responsive to an output of the at least one ultrasound transducer, to: determine a diameter indication of the inflatable medical balloon; and output an indication of the determined diameter indication.

5. The medical balloon sensing assembly of claim 4, wherein the diameter indication comprises a radial diameter of the inflatable medical balloon.

6. The medical balloon sensing assembly of claim 4, wherein the diameter indication comprises a surface topography of the inflatable medical balloon.

7. The medical balloon sensing assembly of claim 4, wherein the diameter indication comprises a protrusion depth of one or more protrusions extending into the inflatable medical balloon.

8. The medical balloon sensing assembly of claim 4, wherein the diameter indication comprises a height-diameter aspect ratio of one or more protrusions extending into the inflatable medical balloon.

9. The medical balloon sensing assembly of claim 4, wherein the diameter indication comprises an indication of the recoil of the inflatable medical balloon.

10. The medical balloon sensing assembly of claim 4, wherein the sensor data unit is further arranged, responsive to the determined diameter indication, to: determine an appropriate orientation for a prosthetic heart valve; and output an indication of the determined orientation.

11. The medical balloon sensing assembly of claim 4, wherein the sensor data unit is further arranged, responsive to the determined diameter indication, to: determine whether implantation of a balloon prosthetic valve at an anatomical location of the inflatable medical balloon is viable; and output an indication of the determined viability.

12. The medical balloon sensing assembly of claim 1, further comprising a prosthetic valve, the inflatable medical balloon positioned within the prosthetic valve such that the prosthetic valve expands responsive to an inflation of the inflatable medical balloon.

13. The medical balloon sensing assembly of claim 1, further comprising: at least one sensor position member; and at least one sensor secured to the at least one sensor position member, wherein the at least one sensor position member is arranged to contact an outer face of the inflatable medical balloon.

14. The medical balloon sensing assembly of claim 1, further comprising: at least one sensor position member; a plurality of force sensors secured to the at least one sensor position member; and a sensor data unit in communication with the plurality of force sensors, wherein the at least one sensor position member is arranged to contact an outer face of the inflatable medical balloon, and wherein the sensor data unit is arranged, responsive to an output of the plurality of force sensors, to: generate a map of forces applied to the inflatable medical balloon; and output an indication of the determined map.

15. The medical balloon sensing assembly of claim 1, further comprising at least one sensor, wherein the inflatable medical balloon exhibits at least one depression, the at least one sensor positioned within the at least one depression.

16. The medical balloon sensing assembly of claim 1, further comprising: at least one force sensor juxtaposed with the inflatable medical balloon; and a sensor data unit in communication with the at least one sensor, wherein, responsive to the output of the at least one force sensor, the sensor data unit is arranged to: determine a force applied to the inflatable medical balloon; and output an indication of the determined force.

17. The medical balloon sensing assembly of claim 1, further comprising: a reservoir containing a predetermined volume of inflation fluid; and a pump in fluid communication with the reservoir and the inflatable medical balloon, wherein the pump is arranged to generate flow of the inflation fluid into the inflatable medical balloon, and wherein, responsive to the output of the at least one ultrasound transducer, the pump is arranged to adjust the flow of the inflation fluid.

18. A medical balloon sensing method, the method comprising: delivering an inflatable medical balloon and a first catheter to a predetermined anatomical location, the inflatable medical balloon secured to the first catheter; and inflating the delivered inflatable medical balloon, wherein at least one ultrasound transducer is positioned on the first catheter within the inflatable medical balloon.

19. The method of claim 18, wherein the at least one ultrasound transducer is directed at a surface of the inflatable medical balloon.

20. The method of claim 18, wherein the at least one ultrasound transducer comprises two ultrasound transducers, an orientation of a first of the ultrasound transducers generally opposing an orientation of the second of the ultrasound transducers.

21. The method of claim 18, further comprising, responsive to an output of the at least one ultrasound transducer: determining a diameter indication of the inflatable medical balloon; and outputting an indication of the determined diameter indication.

22. The method of claim 21, wherein the diameter indication comprises a radial diameter of the inflatable medical balloon.

23. The method of claim 21, wherein the diameter indication comprises a surface topography of the inflatable medical balloon.

24. The method of claim 21, wherein the diameter indication comprises an indication of the recoil of the inflatable medical balloon.

25. The method of claim 18, further comprising, responsive to the output of the at least one ultrasound transducer, controlling a pump to adjust a flow of the inflation fluid from a reservoir into the inflatable balloon.
